SEQUENCE LISTING (11 sequences)

SEQ ID NO: 1 - protein, 303 aa, Homo sapiens
MRAWIFFLLCLAGRALAAPQQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 2 - protein, 91 aa, Homo sapiens
MKVSAAALAVILIATALCAPASASPYSSDTTPCCFAYIARPLPRAHIKEYFYTSGKCSNPAVVFVTRKNRQVCANPEKKWVREYINSLEMS

SEQ ID NO: 3 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 4 - protein, 101 aa, Homo sapiens
MSSAAGFCASRPGLLFLGLLLLPLVVAFASAEAEEDGDLQCLCVKTTSQVRPRHITSLEVIKAGPHCPTAQLIATLKNGRKICLDLQAPLYKKIIKKLLES

SEQ ID NO: 5 - protein, 116 aa, Homo sapiens
MASTVVAVGLTIAAAGFAGRYVLQAMKHMEPQVKQVFQSLPKSAFSGGYYRGGFEPKMTKREAALILGVSPTANKGKIRDAHRRIMLLNHPDKGGSPYIAAKINEAKDLLEGQAKK

SEQ ID NO: 6 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 7 - protein, 249 aa, Homo sapiens
MAARRSQRRRGRRGEPGTALLVPLALGLGLALACLGLLLAVVSLGSRASLSAQEPAQEELVAEEDQDPSELNPQTEESQDPAPFLNRLVRPRRSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 8 - protein, 211 aa, Homo sapiens
MAPLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 9 - protein, 16 aa, Homo sapiens
GGPGAAQLAHLHGILR

SEQ ID NO: 10 - protein, 6 aa, synthetic construct
  REGION 1..6: note = Description of Artificial Sequence: Synthetic 6xHis tag
HHHHHH

SEQ ID NO: 11 - protein, 424 aa, Homo sapiens
MRAPGCGRLVLPLLLLAAAALAEGDAKGLKEGETPGNFMEDEQWLSSISQYSGKIKHWNRFRDEVEDDYIKSWEDNQQGDEALDTTKDPCQKVKCSRHKVCIAQGYQRAMCISRKKLEHRIKQPTVKLHGNKDSICKPCHMAQLASVCGSDGHTYSSVCKLEQQACLSSKQLAVRCEGPCPCPTEQAATSTADGKPETCTGQDLADLGDRLRDWFQLLHENSKQNGSASSVAGPASGLDKSLGASCKDSIGWMFSKLDTSADLFLDQTELAAINLDKYEVCIRPFFNSCDTYKDGRVSTAEWCFCFWREKPPCLAELERIQIQEAAKKKPGIFIPSCDEDGYYRKMQCDQSSGDCWCVDQLGLELTGTRTHGSPDCDDIVGFSGDFGSGVGWEDEEEKETEEAGEEAEEEEGEAGEADDGGYIW

(19) United States
(12) Patent Application Publication
(10) Pub. No.: US 2024/0192227 A1
(43) Pub. Date: Jun. 13, 2024

(54) METHODS OF DIAGNOSING AND PREDICTING RENAL DECLINE

(71) Applicant: Joslin Diabetes Center, Inc., Boston, MA (US)

(72) Inventor: Andrzej Krolewski, Needham, MA (US)

(21) Appl. No.: 18/482,419

(22) Filed: Oct. 6, 2023

Related U.S. Application Data
(63) Continuation of application No. PCT/US22/71640, filed on Apr. 8, 2022.
(60) Provisional application No. 63/215,150, filed on Jun. 25, 2021.
(60) Provisional application No. 63/172,541, filed on Apr. 8, 2021.

(51) Int. Cl. G01N 33/68; A61K 38/18; A61K 38/19; G16H 20/10; G16H 50/30

(52) U.S. Cl. CPC G01N 33/6893; A61K 38/1825; A61K 38/191; G01N 33/6848; G16H 20/10; G16H 50/30; G01N 2333/4709; G01N 2333/4722; G01N 2333/50; G01N 2333/525; G01N 2800/347; G01N 2800/50

(57) ABSTRACT

The present disclosure provides methods for identifying a human subject at risk of developing progressive renal decline by examining a level(s) of a protective protein(s) in a sample from the subject. Level(s) of protein(s) identified in the disclosure are associated with protection against progressive renal failure and end-stage kidney disease (ESKD). Examples of such protective proteins include FGF20, ANGPT1, and TNFSF12.

### RELATED APPLICATIONS

This application claims priority to U.S. Provisional Application No. 63/172,541 filed on Apr. 8, 2021, and claims priority to U.S. Provisional Application No. 63/215,150 filed on Jun. 25, 2021. The entire contents of the foregoing priority applications are incorporated by reference herein.

### GOVERNMENT INTERESTS

This invention was made with Government support under Grant No. DK041526-27 awarded by the National Institutes of Health. The Government may have certain rights in the invention.

### SEQUENCE LISTING

The instant application contains a Sequence Listing which has been submitted electronically in ASCII format and is hereby incorporated by reference in its entirety. Said ASCII copy, created on Apr. 5, 2022, is named J103021_1090WO_SL.txt and is 24,798 bytes in size.

### BACKGROUND OF INVENTION

Chronic kidney disease (CKD) is a slow and progressive loss of kidney function over years of a patient's life. The outcome of progressive renal decline is permanent kidney failure eventually resulting in end-stage renal disease (ESRD; also called end-stage kidney disease ESKD).

Chronic kidney disease is widespread, often associated with other conditions the patient has, such as high blood pressure or diabetes. Unfortunately, renal decline (RD) frequently goes undetected and undiagnosed until the disease is well advanced. As renal failure progresses, the kidney's function becomes severely impaired, resulting in toxic levels of waste building up in the patient. Treatment of chronic kidney disease is aimed at stopping or slowing down the progression of the disease. Chronic renal decline can be devastating to a patient, and may eventually lead to ESKD that will require dialysis and kidney transplant. Identifying patients who are at risk of renal decline would improve early treatment and slow progression of this devastating disease.

### SUMMARY OF THE INVENTION

Given the progressive nature of chronic kidney disease and its severity, identifying patients at risk for progressive renal decline would be beneficial.

The present disclosure is based, at least in part, on the discovery of certain protective proteins whose levels can be used to identify patients/subjects who will be progressing to end-stage kidney disease (ESKD; also referred to herein as end-stage renal disease or ESRD) and those who will be protected.

In a first aspect, the present disclosure provides a method of identifying a human subject at risk of developing progressive renal decline, wherein the method comprises the steps of: detecting a level of at least one protective protein in a sample(s) from a subject in need thereof, wherein the protective protein is selected from the group consisting of fibroblast growth factor 20 (FGF20), angiopoietin-2 (ANGPT1), and tumor necrosis factor ligand superfamily member 12 (TNFSF12); and comparing the level of the protective protein with a reference level of the protective protein, wherein the reference level is a level of the protective protein in a non-progressor human subject. In certain embodiments, the protective protein is Testican-2. In some embodiments, a lower level of the protective protein in comparison to the reference level indicates that the human subject is at risk of developing progressive renal decline, or an equivalent or higher level of the protective protein in comparison to the reference level indicates that the human subject is not at risk of developing progressive renal decline.

In some embodiments of the aforementioned aspect, levels of a combination of protective proteins are detected, wherein the combination of protective proteins is selected from the group consisting of FGF20 and TNFSF12; FGF20 and ANGPT1; and TNFSF12 and ANGPT1; or wherein the combination of protective proteins includes FGF20, TNFSF12, and ANGPT1. In certain embodiments, the combination of detected protective proteins includes Testican-2.

In another aspect, the present disclosure provides a method of identifying a human subject at risk of developing progressive renal decline, wherein the method comprises the steps of: detecting a level of at least one protective protein in a sample(s) from a subject in need thereof, wherein the protective protein is selected from the group consisting of (i) a protective protein from a first group of protective proteins selected from the group consisting of secreted protein acidic and rich in cysteine (SPARC), C-C motif chemokine 5 (CCL5), amyloid beta A4 protein (APP), platelet factor-4 (PF4), and ANGPT1, and/or (ii) a protective protein from a second group of protective proteins selected from the group consisting of DNAJC19 and TNFSF12, and FGF20; and comparing the level of the protective protein with a reference level of the protective protein, wherein the reference level is a level of the protective protein in a non-progressor human subject. In certain embodiments, the protective protein is Testican-2, in combination with one or more protective proteins described herein. In some embodiments, a lower level of the protective protein in comparison to the reference level indicates that the human subject is at risk of developing progressive renal decline, or an equivalent or higher level of the protective protein in comparison to the reference level indicates that the human subject is not at risk of developing progressive renal decline.

In some embodiments of the aforementioned aspect, levels of a combination of protective proteins are detected, wherein the combination of protective proteins is selected from the group consisting of FGF20 and a group 1 protective protein; FGF20 and a group 2 protective protein; a group 1 protective protein and a group 2 protective protein; and FGF20, a group 1 protective protein and a group 2 protective protein. In certain embodiments, the protective protein is Testican-2, in combination with one or more protective proteins described herein. In certain embodiments, the non-progressor is a non-diabetic human subject.

In some embodiments of any of the above aspects, the method further comprises administering a therapy to improve kidney function if the subject is identified as having a risk for progressive renal decline. In one embodiment, an SGLT2 inhibitor is administered to the patient if the patient is identified as being at risk. In some embodiments, the therapy comprises FGF20 (e.g., recombinant FGF20). In some embodiments, the therapy comprises administering to the subject FGF20, an active fragment of FGF20, an FGF20 mimic, or a nucleic acid encoding FGF20, or an active fragment thereof, if the subject is identified as having a risk for progressive renal decline. In other embodiments, the therapy comprises TNFSF12 (e.g., recombinant TNFSF12). In some embodiments, the therapy comprises administering to the subject TNFSF12, an active fragment of TNFSF12, a TNFSF12 mimic, or a nucleic acid encoding TNFSF12, or an active fragment thereof, if the subject is identified as having a risk for progressive renal decline. In yet other embodiments, the therapy comprises ANGPT1 (e.g., recombinant ANGPT1). In some embodiments, the therapy comprises administering to the subject ANGPT1, an active fragment of ANGPT1, an ANGPT1 mimic, or a nucleic acid encoding ANGPT1, or an active fragment thereof, if the subject is identified as having a risk for progressive renal decline. In some embodiments, the therapy comprises administering to the subject Testican-2, an active fragment of Testican-2, a Testican-2 mimic, or a nucleic acid encoding Testican-2, or an active fragment thereof, if the subject is identified as having a risk for progressive renal decline.

In some embodiments, the human subject has impaired kidney function, diabetes, or both. In certain embodiments, the diabetes is type I diabetes or type II diabetes. In other embodiments, the human subject is non-diabetic.

In some embodiments of any of the above aspects, the sample is a plasma sample. In some embodiments, the level of the protective protein is determined using an immunoassay, mass spectrometry, liquid chromatography (LC) fractionation, SOMAscam, Mesoscale platform, or electrochemiluminescence detection. In some embodiments, the immunoassay is an ELISA or a Western blot analysis. In some embodiments, the mass spectrometry matrix assisted laser desorption ionization-time-of-flight (MALDI-TOF), inductively coupled plasma mass spectrometry (ICP-MS), triggered-by-offset, multiplexed, accurate-mass, high-resolution, and absolute quantification (TOMAHAQ), direct analysis in real time mass spectrometry (DART-MS) or secondary ion mass spectrometry (SIMS). In some embodiments, the sample is a blood sample, a serum sample, a plasma sample, a lymph sample, a urine sample, a saliva sample, a tear sample, a sweat sample, a semen sample, a vaginal sample, a bronchial sample, a mucosal sample, or a cerebrospinal fluid (CSF) sample.

In another aspect, the present disclosure provides a protein array for identifying or monitoring progressive renal decline of a human subject, wherein the protein array comprises antibodies or antigen-binding fragments thereof, specific for human FGF20, human TNFSF12 and human ANGPT1.

In yet another aspect, provided herein is a protein array for identifying or monitoring progressive renal decline of a human subject, wherein the protein array comprises antibodies or antigen-binding fragments thereof, specific for human FGF20, human TNFSF12 and human ANGPT1, human SPARC, human CCL5, human APP, human PF4, human ANGPT1, human DNAJC19, human TNFSF12, Testican-2, or combinations thereof.

In another aspect, provided herein is an array comprising a plurality of probes for specifically binding a protein biomarker, wherein the protein biomarker is at least one or more of human FGF20, human TNFSF12, and human ANGPT1.

In yet another aspect, provided herein is an array comprising a plurality of probes for specifically binding a protein biomarker, wherein the protein biomarker is at least one or more of human FGF20, human TNFSF12 and human ANGPT1, human SPARC, human CCL5, human APP, human PF4, human Testican-2, and human DNAJC19.

In another aspect, the present disclosure provides a test panel comprising a protein array as disclosed herein.

In another aspect, the present disclosure provides a kit or assay device comprising a test panel as disclosed herein.

In another aspect, the present disclosure provides a method of inhibiting the progression of progressive renal decline in a human subject, said method comprising administering to a subject an effective amount of at least one protective protein and/or at least one agonist of a protective protein.

In another aspect, the present disclosure provides a method of preventing renal decline in a human subject, said method comprising administering to a subject an effective amount of an agonist of at least one protective protein and/or at least one agonist of a protective protein.

In another aspect, the present disclosure provides a method of treating renal decline in a human subject, said method comprising administering to a subject a therapeutically effective amount of an agonist of at least one protective protein and/or an agonist of at least one protective protein.

In another aspect, provided herein is a method of determining whether a human subject has an increased risk of developing progressive renal disease, the method comprising obtaining a sample from a human subject at risk thereof; detecting the presence of and measuring the level of at least one protective protein in the subject sample; comparing the subject levels of the protective protein with reference levels of the protective protein; determining whether the subject has an increased risk of increased risk of developing progressive renal disease based on the comparison of the subject levels with the reference levels, wherein the presence of the protective protein in the subject sample at levels that are significantly lower than the reference levels indicates that the subject has an increased risk of developing progressive renal disease; and administering a therapy to a subject identified as having a risk of developing progressive renal disease. The method may further comprise monitoring the identified subject for an increase in the protective protein.

In some embodiments of any of the above aspects, the at least one protective protein is one or more of FGF20, TNFSF12, ANGPT1, SPARC, CCL5, APP, PF4, Testican-2, and DNAJC19. In other embodiments, the at least one protective protein is FGF20, an active fragment of FGF20, a FGF20 mimic, or a nucleic acid encoding FGF20, or an active fragment thereof. In various other embodiments, the at least one protective protein is TNFSF12, an active fragment of TNFS12, a TNFSF12 mimic, or a nucleic acid encoding TNFSF12, or an active fragment thereof. In certain other embodiments, the at least one protective protein is ANGPT1, an active fragment of ANGPT1, a ANGPT1 mimic, or a nucleic acid encoding ANGPT1, or an active fragment thereof. In other embodiments, the at least one protective protein is SPARC, an active fragment of SPARC, a SPARC mimic, or a nucleic acid encoding SPARC, or an active fragment thereof. In other embodiments, the at least one protective protein is CCL5, an active fragment of CCL5, a CCL5 mimic, or a nucleic acid encoding CCL5, or an active fragment thereof. In certain other embodiments, the at least one protective protein is APP, an active fragment of APP, a APP mimic, or a nucleic acid encoding APP, or an active fragment thereof. In other embodiments, the at least one protective protein is PF4, an active fragment of PF4, a PF4 mimic, or a nucleic acid encoding PF4, or an active fragment thereof. In other embodiments, the at least one protective protein is DNAJC19, an active fragment of DNAJC19, a DNAJC19 mimic, or a nucleic acid encoding DNAJC19, or an active fragment thereof. In certain embodiments, the at least one protective protein is Testican-2, an active fragment of Testican-2, a Testican-2 mimic, or a nucleic acid encoding Testican-2, or an active fragment thereof.

In yet other embodiments, the nucleic acid is in a vector. In other embodiments, the human subject was previously identified as a progressor at risk of developing progressive renal decline.

In another aspect, the present disclosure provides a method of determining the approximate risk of renal decline in a human subject in a defined time period, the method comprising: a) obtaining a biological sample from the human subject; b) detecting the level of at least one protective protein in the biological sample, wherein the at least one protective protein is selected from the group consisting of FGF20, TNFSF12, ANGPT1, SPARC, CCL5, APP, PF4, Testican-2, and DNAJC19; c) combining data on the level of the protective proteins with clinical data features of the human subject (such as eGFR, uACR, Clinical Chemistry laboratory measurements, vital signs, patient demographics) and d) determining the approximate risk of renal decline (RD) for the human subject as determined using a machine-learned or statistically modelled, prognostic risk-score algorithm (e.g., KidneyIntelX test platform). In certain embodiments, a sample from the human subject is contacted with an antibody, or an antigen binding fragment thereof, that specifically binds to the protective protein and binding of the antibody to the protective protein is measured to determine the level of binding between the protective protein and the antibody.

In some embodiments of any of the above aspects, the method further comprises comparing the level of the at least one protective protein in the biological sample to a non-progressor control level or a normoalbuminuric control level. In some embodiments, the biological sample is obtained from the human subject at a first time point and a second time point. In other embodiments, the second time point is obtained from the human subject about 6 months, about 12 months, about 18 months, about 24 months, about 3 years, about 4 years, about 5 years, about 10 years or about 15 years after the first time point. In certain other embodiments, the method further comprises comparing the level of the at least one protective protein in the biological sample obtained from the human subject at a first time point to the biological sample obtained from the human subject at a second time point.

### BRIEF DESCRIPTION OF THE DRAWINGS

FIGS. 1A-1B provide histograms showing distribution of the top 3 protective protein candidates FGF20, TNFSF12, and ANGPT1 after log 10 transformation. FIG. 1A provides histograms showing distribution of FGF20, TNFSF12, and ANGPT1 after log 10 transformation in the combined T1D discovery and T2D replication cohorts. FIG. 1B provides histograms showing distribution of FGF20, TNFSF12, and ANGPT1 after log 10 transformation in the T1D validation cohort.

FIG. 2 is a graph showing distribution of eGFR slopes (ml/min/1.73 m²/year) in the Joslin Kidney Study cohorts with T1D and T2D. Slow decliners were defined as eGFR loss <3.0 ml/min/1.73 m²/year and fast decliners as eGFR loss ≥3.0 ml/min/1.73 m²/year or ESKD progressors. In each cohort, only ESKD cases that developed during the first 10 years after study entry were considered in the present study. Dashed line indicates eGFR loss equals to 3.0 ml/min/1.73 m²/year.

FIG. 3 is a schematic representation of study design showing the study participants in the exploratory and replication panels and how the candidate protective proteins were selected.

FIGS. 4A-4B provide graphs showing candidate circulating proteins associated with protection against fast progressive renal decline. FIG. 4A is a graph showing Spearman's rank correlation coefficients (rₛ) between baseline concentration of 19 plasma proteins and eGFR slope in the Joslin cohorts with T1D (N=214) and T2D (N=144). Shaded bars are a graphic representation of the effect size. Corresponding two-sided P-values have been provided. *Thresholds for the significance used: FDR adjusted P<0.005 in the T1D exploratory cohort and a nominal P<0.05 in the T2D replication cohort. FIG. 4B is a graph showing odds ratios (95% CI) for the 19 candidate protective proteins and fast progressive renal decline (eGFR loss ≥3.0 ml/min/year) in the combined cohorts with T1D and T2D in univariate and adjusted logistic regression models. The effect is shown as an odds ratio (95% CI) per one quartile increase in circulating baseline concentration of the specific protein. The final model was adjusted for baseline eGFR, HbA1c and ACR with stratification by type of diabetes. The 8 selected markers are in red. PKM2 included in the analysis is based on a previous publication.

FIGS. 5A-5C provide graphs showing association of 8 confirmed protective proteins with clinical covariates and with risk of fast progressive renal decline. FIG. 5A is a graph showing Spearman's rank correlation matrix among 8 candidate protective proteins with TNF-R1 and important clinical covariates in the two cohorts adjusted for type of diabetes. Correlation coefficients (rₛ) are presented as shades of red (positive; marked with #) and blue (negative; marked with ##) which correspond to the magnitude of the effect size. FIG. 5B is a graph showing hierarchical cluster analysis in the combined Joslin cohorts. FIG. 5C is a graph showing odds ratios (95% CI) of covariates selected from a backward selection of covariates using the significance criterion α=0.1. The effects of eGFR and HbA1c on fast progressive renal decline are estimated per 10 ml/min/1.73 m² increase and per 1% increase, respectively. The effect of ACR on fast progressive renal decline is estimated as one-unit increase of log₁₀ ACR. The effect of each protein is shown as an odds ratio (95% CI) per one quartile increase in circulating baseline concentration of the relevant protein. *P<0.05; **P<0.01; ***P<0.001; ****P<0.0001; ns, not significant.

FIG. 6 is a graph Spearman's rank correlation matrix among 11 candidate protective proteins with ACR adjusted for type of diabetes. Correlation coefficients (rₛ) are presented as shades of red (positive) and blue (negative; marked with #) which correspond to the magnitude of the effect size.

FIGS. 7A-7D provide graphs showing the combined effect of protective proteins (FGF20, TNFSF12 and ANGPT1) on risk of fast progressive renal decline and progression to ESKD. FIG. 7A is a graph showing odds ratios for fast progressive renal decline according to index of protection considered as a discrete covariate in the combined exploratory and replication cohorts (N=358) with both types of diabetes and impaired kidney function (also referred to as “renal function”). FIG. 7B is a graph showing cumulative incidence of ESKD (%) according to discrete values of index of protection in the combined exploratory and replication cohorts. FIG. 7C is a graph showing odds ratios for fast progressive renal decline according to index of protection considered as a discrete covariate in the validation cohort (N=294) of T1D subjects with normal kidney function. FIG. 7D is a graph showing cumulative incidence of ESKD (%) according to discrete values of index of protection in the validation cohort. Index of protection: Value above median for each protein was scored as 1 and below as 0; by summing up these scores, a subject could have a total protection index varying between 0 (all proteins below median) and 3 (all proteins above median). *P<0.05; ****P<0.0001; ns, not significant.

FIG. 8 is an extracted ion chromatogram of FGF20 tryptic peptide GGPGAAQLAHLHGILR (SEQ ID NO: 9) (amino acids 50-65). The FGF20 SOMAmer plasma pull-downs in the presence (top) or absence (bottom) of recombinant FGF20.

FIG. 9 provides graphs showing plasma concentrations of exemplar protective proteins ANGPT1 (left panel), TNFSF12 (middle panel), FGF20 (right panel) in the combined Joslin cohorts, for non-progressors and progressors, compared to non-diabetics. Bars depict the mean±standard deviations. One-way ANOVA with Dunn's multiple comparisons test. **P<0.01; ***P<0.001; ****P<0.0001; ns, not significant.

FIG. 10 is a histogram showing the data of comparison of Testican-2 (SPOCK2) plasma levels (RFU) between non-ESKD progressors and ESKD progressors.

### DETAILED DESCRIPTION OF INVENTION

### I. Definitions

Prior to setting forth the invention in detail, definitions of certain terms to be used herein are provided. Unless defined otherwise, all technical and scientific terms used herein have the same meaning as is commonly understood by one of skill in the art.

The term “subject” or “patient,” as used interchangeably herein, refers to a human.

The term “sample” as used herein refers to plasma, serum, cells or tissue obtained from a subject. The source of the tissue or cell sample may be solid tissue (as from a fresh, frozen and/or preserved organ or tissue sample or biopsy or aspirate); whole blood or any blood constituents; or bodily fluids, such as serum, plasma, urine, saliva, sweat or synovial fluid. In one embodiment, the sample is a plasma sample obtained from a human subject.

The term “level” or “amount” of a biomarker, as used herein, refers to the measurable quantity of a biomarker, e.g., protein level of a biomarker. The amount may be either (a) an absolute amount as measured in molecules, moles or weight per unit volume or cells or (b) a relative amount, e.g., measured by densitometric analysis.

As used herein, the term “known standard level”, “reference level” or “control level”, used interchangeably, refers to an accepted or pre-determined level of the biomarker which is used to compare the biomarker level derived from a sample of a patient. In one embodiment, when compared to the reference level of a certain biomarker (protective protein), deviation from the reference level generally indicates either an improvement or deterioration in the disease state or future disease state. In one embodiment, when compared to the reference level of a protective protein, deviation from the reference level generally indicates an increased or decreased likelihood of disease progression in a subject. A reference level can be generated from a sample taken from a healthy (e.g., non-diabetic) individual or from an individual known to have a predisposition to ESKD. In one embodiment, the reference level of a protective protein described herein is the level of the protein in a non-diabetic subject.

As used herein, the term “comparable level” refers to a level of one biomarker that is substantially similar to the level of another, e.g., a control level. In one embodiment, two biomarkers have a comparable level if the level of the biomarker is within one standard deviation of the control biomarker level. In another embodiment, two biomarkers have a comparable level if the level of the biomarker is 20% or less of the level of the control biomarker level.

As used herein, the term “estimated Glomerular Filtration Rate” or “eGFR,” refers to a means for estimating kidney function. In one embodiment, eGFR may be determined based on a measurement of serum creatinine levels. In another embodiment, eGFR may be determined based on a measurement of serum cystatin C levels. In yet another embodiment, eGFR may be determined using the CKD-EPI creatinine equation.

As used herein, the term “a disorder associated with chronic kidney disease” or “a disorder associated with chronic renal disease” refers to a disease or condition associated with impaired kidney function which can cause kidney damage over time. Examples of disorders associated with chronic kidney disease include, but are not limited to, type 1 diabetes, type 2 diabetes, high blood pressure, glomerulonephritis, interstitial nephritis, polycystic kidney disease, prolonged obstruction of the urinary tract (e.g., from conditions such as enlarged prostate, kidney stones and some cancers), vesicoureteral reflux, and recurrent kidney infection. Chronic kidney disease and its stages (CKD 1-5) can usually be characterized or classified accordingly, such as based on the presence of either kidney damage (albuminuria) or impaired estimated glomerular filtration rate (GFR<60 [ml/min/1.73 m²], with or without kidney damage).

As used herein, the term “ESKD progressor”, “progressor” or “rapid progressor” refers to a subject having a disorder associated with chronic kidney disease who has been identified as having an elevated risk for developing ESKD (also referred to herein as ESRD). While an ESKD progressor has a disorder associated with chronic kidney disease, which may put the subject at risk for developing ESKD, the term is meant to include those subjects who have an identified risk elevated above that normally associated with the disorder associated with chronic kidney disease. In one embodiment, a progressor has a level of any one or more of FGF20, TNFSF12, ANGPT1, SPARC, CCL5, APP, PF4, Testican-2, and/or DNAJC19 that is statistically significantly lower than a non-progressor control level or a normoalbuminuric control, and, as such, has an increased risk for developing ESKD. In another embodiment, a progressor has a level of any one or more of FGF20, TNFSF12, and/or ANGPT1 that is statistically significantly lower than a non-progressor control level or a normoalbuminuric control, and, as such, has an increased risk for developing ESKD.

As used herein, the term “non-progressor” refers to a subject having a disorder associated with chronic kidney disease who has a reduced risk of developing ESKD. In one embodiment, a non-progressor is a subject having a disorder associated with chronic kidney disease who is in stage 1 or 2 CKD (Chronic Kidney Disease) but who has a lower risk of progressing to ESKD due, at least in part, to elevated or comparable levels of a protective proteins (e.g., in comparison to a normoalbuminuric control). In one embodiment, a non-progressor is defined as a subject who has a level of any one or more of FGF20, TNFSF12, ANGPT1, SPARC, CCL5, APP, PF4, Testican-2, and/or DNAJC19 that is statistically significantly higher than a progressor control level or is higher or comparable to a normoalbuminuric control. In another embodiment, a non-progressor is defined as a subject who has a level of any one or more of FGF20, TNFSF12, and/or ANGPT1, that is statistically significantly higher than a progressor control level or is higher or comparable to a normoalbuminuric control. In another embodiment, a non-progressor is defined as a subject who has a level of Testican-2, that is statistically significantly higher than a progressor control level or is higher or comparable to a normoalbuminuric control. In one embodiment, a non-progressor is a non-diabetic human subject. Non-diabetic refers to a person who has not been diagnosed with diabetes (Type II).

As used herein, the term “protective protein” refers to a protein whole level in a human subject is associated with renal decline, and/or with an increased or a decreased risk of progressing to ESKD. Protective proteins, as used herein, are proteins whose presence or increased level provides apparent protection against progressive renal decline. Examples of protective proteins include FGF20, TNFSF12, ANGPT1, SPARC, CCL5, APP, PF4, Testican-2, and/or DNAJC19.

As used herein, the term “renal decline” or “RD” (also referred to herein as “kidney decline” (KD)) refers to a condition associated with impaired kidney function. In one embodiment, renal decline is defined as an estimated Glomerular Filtration Rate (eGFR) change of at least −3 ml/min/year (i.e., eGFR loss ≥3.0 ml/min/year). In one embodiment, renal decline is defined as an estimated Glomerular Filtration Rate (eGFR) change of at least −5 ml/min/year (i.e., eGFR loss ≥5.0 ml/min/year). In one embodiment, renal decline is defined as a ≥40% sustained decline in eGFR from baseline (confirmed for at least 3 months).

The term “therapeutically effective amount” or an “effective amount” refers to an amount which, when administered to a living subject, achieves a desired effect on the living subject. The exact amount will depend on the purpose of the treatment, and will be ascertainable by one skilled in the art using known techniques. As is known in the art, adjustments for systemic versus localized delivery, age, body weight, general health, sex, diet, time of administration, drug interaction and the severity of the condition may be necessary, and will be ascertainable with routine experimentation by those skilled in the art. For example, an effective amount of an agent described herein for administration to the living subject is an amount that prevents and/or treats ESKD. For example, for a renal protective agent, a therapeutically effective amount can be an amount that has been shown to provide an observable therapeutic benefit compared to baseline clinically observable signs and symptoms of chronic kidney disease.

As used herein, the term “renal protective agent” refers to an agent that can prevent or delay the progression of nephropathy in a subject having moderately increased albuminuria or diabetic nephropathy. Examples of renal protective agents include, but are not limited to, angiotensin-converting enzyme (ACE) inhibitors and angiotensin—II receptor blockers (ARBs). In one embodiment, a renal protective agent is a protective protein describe herein, or an equivalent there, e.g., an active fragment.

### II. Protective Proteins

The present disclosure is based, at least in part, on the discovery of certain biomarkers whose protein levels can be used to identify subjects/patients who will be progressing to ESKD (also referred to herein as ESRD) and those who will be protected.

Disclosed herein is are methods for identifying whether a human subject is at risk of developing progressive renal decline. The methods include detecting the level of at least one protective protein in a sample(s) from a subject in need thereof. Secreted protein acidic and rich in cysteine (SPARC), C-C motif chemokine 5 (CCL5), amyloid beta A4 protein (APP), platelet factor-4 (PF4), DNAJC19, angiopoietin-2 (ANGPT1), tumor necrosis factor ligand superfamily member 12 (TNFSF12), fibroblast growth factor 20 (FGF20), and Testican-2 (SPOCK2) have been identified by the studies herein as protective proteins whose levels correlate with non-progression of kidney disease. These levels are higher than patients who show progressive disease, and have lower levels of these proteins.

The level of a protective protein or proteins in a sample or samples from a subject can be compared to the level of the protective protein on proteins with a reference level of the protective protein in order to determine the risk of the patient developing progressive renal decline, and eventually ESKD (also referred to herein as ESRD).

Levels of at least one, at least two, at least three, at least four, at least five, at least six, at least seven, or all eight of the protective proteins can be used in the methods disclosed herein.

In one embodiment, a level of each of fibroblast growth factor 20 (FGF20), angiopoietin-2 (ANGPT1), and tumor necrosis factor ligand superfamily member 12 (TNFSF12), or a combination thereof, is compared to a reference level in order to determine the risk of the patient for developing or continuing to have progressive renal decline. In one embodiment, a level of Testican-2 is compared to a reference level in order to determine the risk of the patient for developing or continuing to have progressive renal decline. In another embodiment, levels of each of FGF20 and TNFSF12; FGF20 and ANGPT1; TNFSF12 and ANGPT1; and FGF20, TNFSF12, and ANGPT1, FGF20 and Testican-2; ANGPT1 and Testican-2; TNFSF12 and Testican-2; FGF20, ANGPT1, and Testican-2; ANGPT1, TNFSF12 and Testican-2; FGF20, TNFSF12 and Testican-2; or FGF20, ANGPT1, TNFSF12 and Testican-2 are used in the methods disclosed herein.

In one embodiment, a level of each of fibroblast growth factor 20 (FGF20); a protective protein from a first group of protective proteins including SPARC, CCL5, APP, PF4 and ANGPT1 (Group 1 protective proteins); a protective protein from a second group of protective proteins including DNAJC19 and TNFSF12 (Group 2 protective proteins), or combinations thereof, e.g., a group 1 and a group 2 protective protein, or FGF20 and either a group 1 or a group 2 protective protein, is compared to a reference level in order to determine the risk of the patient for developing or continuing to have progressive renal decline.

A table describing the nine protective proteins
identified herein is provided below:

| Protective Protein Full Name | UniProt ID | Gene Symbol |
|---|---|---|
| Tumor necrosis factor ligand superfamily | O43508 | TNFSF12 |
| member 12 | | |
| Secreted protein acidic and rich in cysteine | P09486 | SPARC |
| C-C motif chemokine 5 | P13501 | CCL5 |
| Amyloid beta A4 protein | P05067 | APP |
| Platelet factor 4 | P02776 | PF4 |
| Fibroblast growth factor 20 | Q9NP95 | FGF20 |
| Angiopoietin-1 | Q15389 | ANGPTI |
| DnaJ Heat Shock Protein Family Member | Q96DA6 | DNAJC19 |
| C19 | | |
| Testican-2 | Q92563 | SPOCK2 |

Once the protective protein level is detected in a sample from the subject, the level is compared to a reference level in order to determine whether the level coincides with a progressor profile (risk) or a non-progressor (protection).

The onset of progressive renal decline begins when patients have normal kidney function and it progresses almost linearly to ESKD, although the rate of decline expressed as the slope of the estimated glomerular filtration rate (eGFR) varies among those individuals ranging from −72 to 3.0 ml/min/year.

In one embodiment, the reference level of a protective protein is a level of a non-diabetic human subject, wherein a lower level of the protective protein in comparison to the reference level indicates that the human subject is at risk of developing progressive renal decline. Alternatively, equivalent or higher level of the protective protein in comparison to the reference level indicates that the human subject is not at risk of developing progressive renal decline.

In one embodiment, the human subject who provides the sample for testing is a subject who has a condition associated with progressive renal decline, such as diabetes or high blood pressure. In another embodiment, the subject may have impaired kidney function, where determining the risk of further renal decline would be desirable to mitigate kidney destruction. In one embodiment, the subject has type I diabetes or type II diabetes.

For subjects with diabetes, the risk of chronic kidney disease and ESKD remains relatively high despite improvements in glycemic control and advances in reno-protective therapies over the last 20 years for the prevention and treatment of DKD (Rosolowsky et al., J Am Soc Nephrol 22: 545-553 (2011); de Boer et al., JAMA 305: 2532-2539 (2011)). Findings from Joslin Kidney Study, a longitudinal study of more than 3000 subjects with diabetes, demonstrate that progressive renal decline is the major clinical manifestation of DKD that underlies progression to ESKD (Perkins et al., N Engl J Med 348: 2285-2293 (2003); Perkins et al., J Am Soc Nephrol 18: 1353-1361 (2007); Krolewski, Diabetes Care 38, 954-962 (2015); Krolewski et al., Kidney International 91: 1300-1311 (2017)).

The incidence of ESKD in diabetes patients continues to increase despite improvements in glycemic control and advances in reno-protective therapies, which are almost universally implemented.

Diabetic kidney disease (DKD) and its important clinical manifestation, progressive renal decline that leads to end-stage kidney disease (ESKD; also referred to herein as ESRD), is a major health burden for subjects with diabetes. The disease process that underlies progressive renal decline comprises factors/pathways that increase risk of this outcome as well as factors/pathways that protect against progressive renal decline. Using an untargeted proteomic profiling of circulating proteins from subjects in three independent cohorts with longstanding Type 1 and Type 2 diabetes and varying stages of DKD followed for 7-15 years has identified 3 elevated plasma proteins, fibroblast growth factor 20 (FGF20; OR=0.69; 95% CI: 0.54-0.88), angiopoietin-1 (ANGPT1; OR=0.72; 95% CI: 0.57-0.91) and tumor necrosis factor ligand superfamily member 12 (TNFSF12; OR=0.75; 95% CI: 0.59-0.95), that were associated with protection against progressive renal decline and progression to ESKD. The combined effect of these 3 protective proteins was well demonstrated by very low cumulative risk of ESKD in subjects who had high baseline concentrations (above median) for all 3 proteins, whereas the cumulative risk of ESKD was high in subjects with low concentrations (below median) of these proteins at the beginning of follow-up. This protective effect was manifested strongly and independently from circulating inflammatory proteins and important clinical covariates, and was confirmed in an independent cohort of diabetic subjects with normal kidney function. The three protective proteins may serve as biomarkers to stratify diabetic subjects according to risk of progression to ESKD.

In one embodiment, the sample tested from the subject is a plasma sample. Multiple samples may be used in testing one or more protective proteins. Alternatively, one sample can be used to test one or more protective proteins.

Detection of the protective proteins can be determined according to standard immunoassays. For example, ELISA or electrochemiluminescence detection (e.g., Meso Sector S600 (Meso Scale Diagnostics)).

Also included herein is a protein array for identifying or monitoring progressive renal decline of a human subject. In one embodiment, said protein array comprises antibodies or antigen-binding fragments thereof, specific for human FGF20, human TNFSF12, human ANGPT1, and/or human Testican-2.

In another embodiment, the disclosure provides a protein array for identifying or monitoring progressive renal decline of a human subject, said protein array comprising antibodies or antigen-binding fragments thereof, specific for human FGF20, human TNFSF12 and human ANGPT1, human SPARC, human CCL5, human APP, human PF4, human DNAJC19, human Testican-2, or combinations thereof.

In one embodiment, an array comprises a plurality of probes for specifically binding a protein biomarker, wherein the protein biomarker is at least one or more of human FGF20, human TNFSF12 and human ANGPT1.

In one embodiment, an array comprises a plurality of probes for specifically binding a protein biomarker, wherein the protein biomarker is at least one or more of human FGF20, human TNFSF12, human ANGPT1, human SPARC, human CCL5, human APP, human PF4, human DNAJC19, human Testican-2.

The studies described herein identify nine protective proteins (i.e., secreted protein acidic and rich in cysteine (SPARC), C-C motif chemokine 5 (CCL5), amyloid beta A4 protein (APP), platelet factor-4 (PF4), DNAJC19, angiopoietin-2 (ANGPT1), tumor necrosis factor ligand superfamily member 12 (TNFSF12), fibroblast growth factor 20 (FGF20), and Testican-2, that can be used to identify patients, according to levels in a sample, who are likely to develop ESKD or have continued progressive kidney disease leading to ESKD or will be protected against progression to ESKD.

### SPARC

A protective protein of the present disclosure is Secreted Protein Acidic and Cysteine Rich (SPARC).

The terms “Secreted Protein Acidic and Cysteine Rich” gene, or “SPARC” gene, also known as “Osteonectin,” “ONT,” “Basement-Membrane Protein 40,” “BM-40 and “OI17,” refers to the gene that is expressed at high levels in tissues undergoing morphogenesis, remodeling and wound repair. The SPARC gene encodes for a protein called SPARC. SPARC is a 32-35 kD Ca2+-binding matricellular glycoprotein whose modular organization is phylogenetically conserved (Martinek, et al. Dev. Genes Evol. 212: 124-133.) SPARC binds to collagen type I in the extracellular space (Mendozo-Londono, et al. Am J Hum Genet. 2015 Jun. 4; 96(6): 979-985.) Biochemical studies indicate that SPARC binds to several collagenous and non-collagenous ECM molecules, including a Ca2+-dependent interaction with network-forming collagen IV. SPARC protein comprises three domains, a Follistin-like domain, a Kazal like domain and an EF hand domain, and comprises two calcium binding sites. The Follistin like acidic domain binds 5 to 8 Ca²⁺ with a low affinity and an EF-hand loop binds a Ca²⁺ ion with a high affinity. In bone, SPARC is expressed by osteoblasts. SPARC-null mice develop progressive osteoporosis, due to a defect in bone formation (Delany, et al. J. Clin. Invest. 2000; 105: 915-923).

SPARC polymorphisms, particularly the polymorphism in the 3′ UTR influences SPARC accumulation in bone, and is associated with variations in bone formation, variations in bone mass, and may play a role in the pathogenesis of osteoporosis in adults (Delany, et al. (2016) Osteoporos. Int. 2008; 19: 969-978; Dole, et al. (2016) J. Bone Miner. Res. 2015; 30:723-732). Homozygous mutations in SPARC can give rise to severe bone fragility in humans (Mendozo-Londono, et al. Am J Hum Genet. 2015 Jun. 4; 96(6): 979-985.)

The nucleotide sequence of the genomic region of human chromosome harboring the SPARC gene may be found in, for example, the Genome Reference Consortium Human Build 38 (also referred to as Human Genome build 38 or GRCh38) available at GenBank. The nucleotide sequence of the genomic region of human chromosome 5 harboring the SPARC gene may also be found at, for example, GenBank Accession No. NC_000005.10, corresponding to nucleotides 151,661,096-151,686,975 of human chromosome 5. Three transcript variants encoding different isoforms have been found for this gene. Exemplary nucleotide and amino acid sequences of SPARC can be found, for example, at GenBank Accession No. NM_003118.4 (Homo sapiens SPARC transcript variant 1). Amino acid sequence of human SPARC transcript variant 1 is provided below:

(SEQ ID NO: 1)
MRAWIFFLLCLAGRALAAPQQEALPDETEVVEETVAEVTEVSVGANP
VQVEVGEFDDGAEETEEEVVAENPCQNHHCKHGKVCELDENNTPMCV
CQDPTSCPAPIGEFEKVCSNDNKTFDSSCHFFATKCTLEGTKKGHKL
HLDYIGPCKYIPPCLDSELTEFPLRMRDWLKNVLVTLYERDEDNNLL
TEKQKLRVKKIHENEKRLEAGDHPVELLARDFEKNYNMYIFPVHWQF
GQLDQHPIDGYLSHTELAPLRAPLIPMEHCTTRFFETCDLDNDKYIA
LDEWAGCFGIKQKDIDKDLVI

Further examples of SPARC sequences can be found in publicly available databases, for example, GenBank, OMIM, and UniProt (P09486). Additional information on SPARC can be found, for example, at the NCBI web site that refers to gene 6678. The term SPARC as used herein also refers to variations of the SPARC gene including variants provided in the clinical variant database, for example, at the NCBI clinical variants web site that refers to the term NM_003118.4.

### CCL5

A protective protein of the present disclosure is C-C Motif Chemokine Ligand 5 (CCL5).

The terms “C-C Motif Chemokine Ligand 5” gene, or “CCL5” gene, also known as “RANTES,” “SCYA5,” “SISd,” “EoCP” and “D17S136E,” refers to the gene that encodes a CCL5 protein, a chemotactic for T cells, eosinophils, and basophils, that plays an active role in recruiting leukocytes into inflammatory sites. The CCL5 protein is a 8 kD protein with a single domain. CCL5 is a chemoattractant for blood monocytes, memory T-helper cells and eosinophils. CCL5 causes the release of histamine from basophils and activates eosinophils and is known to activate several chemokine receptors including CCR1, CCR3, CCR4 and CCR5. CCL5 and one of its cognate receptors, CCR5 are best known as one of the major HIV-suppressive factors produced by CD8+ T-cells and recombinant CCL5 protein induces a dose-dependent inhibition of different strains of HIV-1, HIV-2, and simian immunodeficiency virus (SIV). CCL5 activates T cells when in high concentration through a tyrosine kinase pathway (Wong et al. J Biol Chem 273:309-314 (1998); Bacon et al. Science 269:1727-1730 (1995)) leads to production of IFNγ by T cells (Appay et al. Int Immunol 12:1173-1182 (2000)) and is thought to induce maturation of dendritic cells (Fischer, et al. J Immunol 167:1637-1643 (2001)). High levels of CCL5 protein was demonstrated in synovial CD8+ T cells, from which it is rapidly released on T cell receptor triggering (Pharoah et al. Arthritis Res Ther 8(2): R50 (2006)) CCL5 signals directly on cancer cells to promote survival, invasion, and stem cell renewal. In breast cancer, CCL5 expressed by MSCs act on breast cancer cells to promote invasion and metastasis (Karnoub et al. Nature 449(7162):557-63 (2007)).

The nucleotide sequence of the genomic region of human chromosome harboring the CCL5 gene may be found in, for example, the Genome Reference Consortium Human Build 38 available at GenBank. CCL5 gene is one of several chemokine genes clustered on the q-arm of chromosome 17. The nucleotide sequence of the genomic region of human chromosome 17 harboring the CCL5 gene may also be found at, for example, GenBank Accession No. NC_000017.11, corresponding to nucleotides 35871491-35880360 of human chromosome 17. Four transcript variants encoding different isoforms have been found for this gene. Exemplary nucleotide and amino acid sequences of CCL5 can be found, for example, at GenBank Accession No. NM_002985.3 (Homo sapiens CCL5 transcript variant 1). Amino acid sequence of human CCL5 transcript variant 1 is provided below:

(SEQ ID NO: 2)
MKVSAAALAVILIATALCAPASASPYSSDTTPCCFAYIARPLPRAHI
KEYFYTSGKCSNPAVVFVTRKNRQVCANPEKKWVREYINSLEMS

Further examples of CCL5 sequences can be found in publicly available databases, for example, GenBank, OMIM, and UniProt (P13501). Additional information on CCL5 can be found, for example, at the NCBI web site that refers to gene 6352. The term CCL5 as used herein also refers to variations of the CCL5 gene including variants provided in the clinical variant database, for example, at the NCBI clinical variants web site that refers to the term NM_002985.3.

### APP

Another protective protein of the present disclosure is Amyloid Beta Precursor Protein (APP).

The terms “Amyloid Beta Precursor Protein” gene, or “APP” gene, also known as “ABPP,” “A4,” “AD1,” “Peptidase Nexin-II” and “PreA4,” refers to the gene that encodes a Amyloid Beta A4 protein. APP is a type I transmembrane protein with a short cytoplasmic tail and a large ectodomain, including copper-binding sites in its E1 and E2 domains (Kong et al. Eur Biophys J 37(3):269-79 (2008); Dahms et al. J Mol Biol 416(3):438-52 (2012)). APP protein plays a central role in Alzheimer's pathogenesis (Masters et al. Brain 129(Pt 11):2823-39 (2006)). APP is also essential in synaptic processes, including trans-cellular synaptic adhesion as a cell surface receptor, neurite growth, neuronal adhesion, axonogenesis, synaptogenesis, promotion of cell mobility and transcription regulation through protein-protein interactions (Müller et al. Cold Spring Harb Perspect Med 2(2):a006288 (2012)). App is implicated in copper homeostasis/oxidative stress through copper ion reduction. In vitro, copper-metallated APP induces neuronal death directly or is potentiated through Cu²⁺-mediated low-density lipoprotein oxidation (White et al. J Neurosci 19(21):9170-9 (1999); Maynard et al. J Biol Chem 277(47):44670-6 (2002)). APP knock-out mice show cognitive deficits, and inactivation of APP on the APLP2 knock-out background in either the presynaptic or postsynaptic compartment caused defects in the neuromuscular synapse (Müller et al. Cold Spring Harb Perspect Med 2(2):a006288(2012)).

The nucleotide sequence of the genomic region of human chromosome harboring the APP gene may be found in, for example, the Genome Reference Consortium Human Build 38 available at GenBank. The nucleotide sequence of the genomic region of human chromosome 21 harboring the APP gene may also be found at, for example, GenBank Accession No. NC_000021.9, corresponding to nucleotides 25880550-26171128 of human chromosome 21. Multiple transcript variants encoding different isoforms have been found for this gene. Exemplary nucleotide and amino acid sequences of APP can be found, for example, at GenBank Accession No. NM_000484.4 (Homo sapiens APP transcript variant 1). Amino acid sequence of human APP transcript variant 1 is provided below:

(SEQ ID NO: 3)
MLPGLALLLLAAWTARALEVPTDGNAGLLAEPQIAMFCGRLNMHMNV
QNGKWDSDPSGTKTCIDTKEGILQYCQEVYPELQITNVVEANQPVTI
QNWCKRGRKQCKTHPHFVIPYRCLVGEFVSDALLVPDKCKFLHQERM
DVCETHLHWHTVAKETCSEKSTNLHDYGMLLPCGIDKFRGVEFVCCP
LAEESDNVDSADAEEDDSDVWWGGADTDYADGSEDKVVEVAEEEEVA
EVEEEEADDDEDDEDGDEVEEEAEEPYEEATERTTSIATTTTTTTES
VEEVVREVCSEQAETGPCRAMISRWYFDVTEGKCAPFFYGGCGGNRN
NFDTEEYCMAVCGSAMSQSLLKTTQEPLARDPVKLPTTAASTPDAVD
KYLETPGDENEHAHFQKAKERLEAKHRERMSQVMREWEEAERQAKNL
PKADKKAVIQHFQEKVESLEQEAANERQQLVETHMARVEAMLNDRRR
LALENYITALQAVPPRPRHVENMLKKYVRAEQKDRQHTLKHFEHVRM
VDPKKAAQIRSQVMTHLRVIYERMNQSLSLLYNVPAVAEEIQDEVDE
LLQKEQNYSDDVLANMISEPRISYGNDALMPSLTETKTTVELLPVNG
EFSLDDLQPWHSFGADSVPANTENEVEPVDARPAADRGLTTRPGSGL
TNIKTEEISEVKMDAEFRHDSGYEVHHQKLVFFAEDVGSNKGAIIGL
MVGGVVIATVIVITLVMLKKKQYTSIHHGVVEVDAAVTPEERHLSKM
QQNGYENPTYKFFEQMQN

Further examples of APP sequences can be found in publicly available databases, for example, GenBank, OMIM, and UniProt (P05067). Additional information on APP can be found, for example, at the NCBI web site that refers to gene 351. The term APP as used herein also refers to variations of the APP gene including variants provided in the clinical variant database, for example, at the NCBI clinical variants web site that refers to the term NM_000484.4.

### PF4

A protective protein of the present disclosure is platelet factor-4 (PF4).

The terms “platelet factor-4” gene, or “PF4” gene, also known as “CXCL4,” “Chemokine (C-X-C Motif) Ligand 4,” “Oncostatin-A,” “SCYB4” and “Iroplact,” refers to the gene that encodes a PF4 protein. PF4 is a chemokine primarily released from the alpha granules of activated platelets in the form of a homo-tetramer which has high affinity for heparin and is involved in platelet aggregation. PF4 is known to be secreted by a variety of immune cells (Levine et al. J Biol Chem 251(2):324-8 (1976); Bon et al. N Engl J Med 370(5):433-43 (2014)). PF4 is chemotactic for numerous other cell types and also functions as an inhibitor of hematopoiesis, angiogenesis and T-cell function. The protein also exhibits antimicrobial activity against Plasmodium falciparum. PF4 has also been implicated in the pathology of a variety of inflammatory diseases including myelodysplastic syndromes, malaria, HIV-1, atherosclerosis, inflammatory bowel disease, and rheumatoid arthritis (Affandi et al. Eur J Immunol 48(3):522-531 (2018); Yeo et al. Ann Rheum Dis 75(4):763-71 (2016)).

The nucleotide sequence of the genomic region of human chromosome harboring the APP gene may be found in, for example, the Genome Reference Consortium Human Build 38 available at GenBank. The nucleotide sequence of the genomic region of human chromosome 4 harboring the PF4 gene may also be found at, for example, GenBank Accession No. NC_000004.12, corresponding to nucleotides 73,980,811-73,982,027 of human chromosome 4. This gene has one identified transcript. Exemplary nucleotide and amino acid sequences of PF4 can be found, for example, at GenBank Accession No. NM_002619.4 (Homo sapiens PF4 transcript variant 1). Amino acid sequence of human PF4 transcript variant 1 is provided below:

(SEQ ID NO: 4)
MSSAAGFCASRPGLLFLGLLLLPLVVAFASAEAEEDGDLQCLCVKTT
SQVRPRHITSLEVIKAGPHCPTAQLIATLKNGRKICLDLQAPLYKKI
IKKLLES

Further examples of PF4 sequences can be found in publicly available databases, for example, GenBank, OMIM, and UniProt (P02776). Additional information on PF4 can be found, for example, at the NCBI web site that refers to gene 5196. The term PF4 as used herein also refers to variations of the PF4 gene including variants provided in the clinical variant database, for example, at the NCBI clinical variants web site that refers to the term NM_002619.4.

### DNAJC19

A protective protein of the present disclosure is DnaJ Heat Shock Protein Family (Hsp40) Member C19 (DNAJC19).

The terms “DnaJ Heat Shock Protein Family (Hsp40) Member C19” gene, or “DNAJC19” gene, also known as “TIMM14,” “TIM14,” “PAM18,” and “Mitochondrial Import Inner Membrane Translocase Subunit TIM14,” refers to the gene that encodes a DNAJC19 protein. The DNAJC19 protein is a 6.29 kDa protein composed of 59 amino acids possessing an unusual structure compared to the rest of the DNAJ protein family. The DNAJ domain of DNAJC19 is located at the C-terminal rather than the N-terminal, and the transmembrane domain confers membrane-bound localization for DNAJC19 while other DNAJ proteins are cytosolic (Zong et al. Circulation Research 113 (9): 1043-53). DNAJC19 is required for the ATP-dependent import of mitochondrial pre-proteins into the mitochondrial matrix. The J-domain of DNAJC19 stimulates mtHsp70 ATPase activity to power this transport (Mokranjac et al. EMBO J 22 (19): 4945-56). Defects in DNAJC19 have been associated with dilated cardiomyopathy with ataxia (DCMA), growth failure, microcytic anemia, and male genital anomalies. DNAJC19 was first implicated in DCMA in a study on the consanguineous Hutterite population, which has since been confirmed in other European populations (Ojala et al. Pediatric Research 72 (4): 432-7). In the clinic, DNAJC19 mutations were detected by screening for elevated levels of 3-methylglutaconic acid, mitochondrial distress, dilated cardiomyopathy, prolongation of the QT interval in the electrocardiogram, and cerebellar ataxia (Ojala et al. Pediatric Research 72 (4): 432-7; Koutras et al. Frontiers in Cellular Neuroscience 8: 191).

The nucleotide sequence of the genomic region of human chromosome harboring the DNAJC19 gene may be found in, for example, the Genome Reference Consortium Human Build 38 available at GenBank. The nucleotide sequence of the genomic region of human chromosome 3 harboring the DNAJC19 gene may also be found at, for example, GenBank Accession No. NC_000003.12, corresponding to nucleotides 180983709-180989838 of human chromosome 3. Exemplary nucleotide and amino acid sequences of DNAJC19 can be found, for example, at GenBank Accession No. NM_145261.4 (Homo sapiens DnaJ heat shock protein family (Hsp40) member C19 (DNAJC19) transcript variant 1). Amino acid sequence of human DNAJC19 is provided below:

(SEQ ID NO: 5)
MASTVVAVGLTIAAAGFAGRYVLQAMKHMEPQVKQVFQSLPKSAFSG
GYYRGGFEPKMTKREAALILGVSPTANKGKIRDAHRRIMLLNHPDKG
GSPYIAAKINEAKDLLEGQAKK

Further examples of DNAJC19 sequences can be found in publicly available databases, for example, GenBank, OMIM, and UniProt (Q96DA6). Additional information on DNAJC19 can be found, for example, at the NCBI web site that refers to gene 131118. The term DNAJC19 as used herein also refers to variations of the DNAJC19 gene including variants provided in the clinical variant database, for example, at the NCBI clinical variants web site that refers to the term NM_145261.4.

### ANGPT1

A protective protein of the present disclosure is Angiopoietin 1 (ANGPT1).

The terms “Angiopoietin 1” gene, or “ANGPT1” gene, also known as “KIAA0003,” “ANG-1,” “AGP1,” and “AGPT,” refers to the gene that encodes a ANGPT1 protein. ANGPT1 is a secreted 70-kDa glycoprotein and a member of the angiopoietin family of growth factors. ANGPT1 is the major agonist for the tyrosine kinase receptor, Tek, which is found primarily on endothelial cells. ANGPT1 is produced by vasculature support cells and specialized pericytes such as podocytes in the kidney and ITO cells in the liver (Satchell et al. J Am Soc Nephrol 13(2):544-550 (2002)). ANGPT1 plays an important role in the regulation of angiogenesis, endothelial cell survival, proliferation, migration, adhesion and cell spreading, reorganization of the actin cytoskeleton, and maintenance of vascular quiescence (Jeansson et al. J Clin Invest 121(6): 2278-2289 (2011)). The ANGPT1/Tek pathway is critical for normal development, as conventional ANGPT1 or Tek knockout mice exhibit lethality between E9.5 and E12.5, with similar abnormal vascular phenotypes and loss of heart trabeculations (Suri et al. Cell 87(7):1171-80 (1996); Tachibana et al. Mol Cell Biol 25(11):4693-702 (2005)).

The nucleotide sequence of the genomic region of human chromosome harboring the ANGPT1 gene may be found in, for example, the Genome Reference Consortium Human Build 38 available at GenBank. The nucleotide sequence of the genomic region of human chromosome 8 harboring the ANGPT1 gene may also be found at, for example, GenBank Accession No. NC_000008.11, corresponding to nucleotides 107249482-107497918 of human chromosome 8. Exemplary nucleotide and amino acid sequences of ANGPT1 can be found, for example, at GenBank Accession No. NM_001146.5 (Homo sapiens angiopoietin 1 (ANGPT1), transcript variant 1). Amino acid sequence of human ANGPT1 is provided below:

(SEQ ID NO: 6)
MTVFLSFAFLAAILTHIGCSNQRRSPENSGRRYNRIQHGQCAYTFIL
PEHDGNCRESTTDQYNTNALQRDAPHVEPDFSSQKLQHLEHVMENYT
QWLQKLENYIVENMKSEMAQIQQNAVQNHTATMLEIGTSLLSQTAEQ
TRKLTDVETQVLNQTSRLEIQLLENSLSTYKLEKQLLQQTNEILKIH
EKNSLLEHKILEMEGKHKEELDTLKEEKENLQGLVTRQTYIIQELEK
QLNRATTNNSVLQKQQLELMDTVHNLVNLCTKEGVLLKGGKREEEKP
FRDCADVYQAGFNKSGIYTIYINNMPEPKKVFCNMDVNGGGWTVIQH
REDGSLDFQRGWKEYKMGFGNPSGEYWLGNEFIFAITSQRQYMLRIE
LMDWEGNRAYSQYDRFHIGNEKQNYRLYLKGHTGTAGKQSSLILHGA
DFSTKDADNDNCMCKCALMLTGGWWFDACGPSNLNGMFYTAGQNHGK
LNGIKWHYFKGPSYSLRSTTMMIRPLDF

Further examples of ANGPT1 sequences can be found in publicly available databases, for example, GenBank, OMIM, and UniProt (Q15389). Additional information on ANGPT1 can be found, for example, at the NCBI web site that refers to gene 284. The term ANGPT1 as used herein also refers to variations of the ANGPT1 gene including variants provided in the clinical variant database, for example, at the NCBI clinical variants web site that refers to the term NM_001146.5.

### TNFSF12

A protective protein of the present disclosure is Tumor Necrosis Factor Superfamily Member 12 (TNFSF12).

The terms “Tumor Necrosis Factor Superfamily Member 12” gene, or “TNFSF12” gene, also known as “APO3L,” “DR3LG,” “TWEAK,” and “TNLG4A,” refers to the gene that encodes a TNFSF12 protein. TNFSF12 is a member of the tumor necrosis factor (TNF) family of proteins that play pivotal roles in the regulation of the immune system. TNFSF12 is expressed widely in many tissues and induces interleukin-8 synthesis in a number of cell lines (Chicheportiche et al. Cell Biology and Metabolism 272(51): 32401-32410 (1997)). The human adenocarcinoma cell line, HT29, underwent apoptosis in the presence of both TNFSF12 and interferon-7. Leukocytes are the main source of TNFSF12 including human resting and activated monocytes, dendritic cells and natural killer cells (Maecker et al. Cell 123(5): 931-44). TNFSF12 suppresses production of IFN-γ and IL-12, curtailing the innate response and its transition to adaptive TH1 immunity. TNFSF12 also promotes proliferation and migration of endothelial cells, acting as a regulator of angiogenesis.

The nucleotide sequence of the genomic region of human chromosome harboring the TNFSF12 gene may be found in, for example, the Genome Reference Consortium Human Build 38 available at GenBank. The nucleotide sequence of the genomic region of human chromosome 17 harboring the TNFSF12 gene may also be found at, for example, GenBank Accession No. NC_000017.11, corresponding to nucleotides 7549058-7557881 of human chromosome 17. Exemplary nucleotide and amino acid sequences of TNFSF12 can be found, for example, at GenBank Accession No. NM_003809.3 (Homo sapiens TNF superfamily member 12 (TNFSF12), transcript variant 1). Amino acid sequence of human TNFSF12 is provided below:

(SEQ ID NO: 7)
MAARRSQRRRGRRGEPGTALLVPLALGLGLALACLGLLLAVVSLGSR
ASLSAQEPAQEELVAEEDQDPSELNPQTEESQDPAPFLNRLVRPRRS
APKGRKTRARRAIAAHYEVHPRPGQDGAQAGVDGTVSGWEEARINSS
SPLRYNRQIGEFIVTRAGLYYLYCQVHFDEGKAVYLKLDLLVDGVLA
LRCLEEFSATAASSLGPQLRLCQVSGLLALRPGSSLRIRTLPWAHLK
AAPFLTYFGLFQVH

Further examples of TNFSF12 sequences can be found in publicly available databases, for example, GenBank, OMIM, and UniProt (043508). Additional information on TNFSF12 can be found, for example, at the NCBI web site that refers to gene 8742. The term TNFSF12 as used herein also refers to variations of the TNFSF12 gene including variants provided in the clinical variant database, for example, at the NCBI clinical variants web site that refers to the term NM_003809.3.

### FGF20

Another protective protein of the present disclosure is Fibroblast Growth Factor 20 (FGF20).

The terms “Fibroblast Growth Factor 20” gene, or “FGF20” gene, also known as “RHDA2,” refers to the gene that encodes a FGF20 protein. FGF20 is primarily expressed in normal brain, particularly the cerebellum. The rat homolog is preferentially expressed in the brain and able to enhance the survival of midbrain dopaminergic neurons in vitro. FGF20 is a member of the of the fibroblast growth factor (FGF) family that possess broad mitogenic and cell survival activities, and are involved in a variety of biological processes, including cell growth, morphogenesis, tissue repair, tumor growth, invasion and embryonic development (Koga et al. Biochemical and Biophysical Research Communications 261(3): 756-65). Gene polymorphisms of FGF20 has been implicated in Parkinson's disease (Zhao et al. Neurol Sci 37(7):1119-26 (2016); Zhu et al. Neurol Sci 35(12) (2014)).

The nucleotide sequence of the genomic region of human chromosome harboring the FGF20 gene may be found in, for example, the Genome Reference Consortium Human Build 38 available at GenBank. The nucleotide sequence of the genomic region of human chromosome 8 harboring the FGF20 gene may also be found at, for example, GenBank Accession No. NC_000008.11, corresponding to nucleotides 16992181-17002345 of human chromosome 8. Exemplary nucleotide and amino acid sequences of FGF20 can be found, for example, at GenBank Accession No. NM_019851.3 (Homo sapiens fibroblast growth factor 20 (FGF20)). Amino acid sequence of human FGF20 is provided below:

(SEQ ID NO: 8)
MAPLAEVGGFLGGLEGLGQQVGSHFLLPPAGERPPLLGERRSAAERS
ARGGPGAAQLAHLHGILRRRQLYCRTGFHLQILPDGSVQGTRQDHSL
FGILEFISVAVGLVSIRGVDSGLYLGMNDKGELYGSEKLTSECIFRE
QFEENWYNTYSSNIYKHGDTGRRYFVALNKDGTPRDGARSKRHQKFT
HFLPRPVDPERVPELYKDLLMYT

Further examples of FGF20 sequences can be found in publicly available databases, for example, GenBank, OMIM, and UniProt (Q9NP95). Additional information on FGF20 can be found, for example, at the NCBI web site that refers to gene 26281. The term FGF20 as used herein also refers to variations of the FGF20 gene including variants provided in the clinical variant database, for example, at the NCBI clinical variants web site that refers to the term NM_019851.3.

### Testican-2

Another protective protein that can be used as a marker in the methods and compositions described herein is Testican-2.

Human Testican-2 protein is encoded by the SPOCK2 gene, also known as TICN2 or KIAA0275. Testican-2 binds with glycosaminoglycans to form part of the extracellular matrix. The protein contains thyroglobulin type-1, follistatin-like, and calcium-binding domains, and has glycosaminoglycan attachment sites in the acidic C-terminal region. SPOCK (SPARC/osteonectin CWCV and Kazal-like domains) encodes a secreted proteoglycan with three known homologs, SPOCK1, -2, and -3. SPOCK was initially characterized as a progenitor form of a seminal plasma GAG-bearing peptide and was later cloned and identified as a chondroitin/heparan sulfate proteoglycan (HSPG). The SPOCK1 and -2 proteoglycans inhibit neuronal cell attachment and neurite extension. Moreover, polymorphism in SPOCK2 was recently identified as a genetic trait linked to susceptibility to bronchopulmonary dysplasia, a chronic respiratory disease common among premature infants (Hadchouel et al., Am J Respir Crit Care Med., 2011, 184(10):1164-70), and functions as a protective barrier against virus infection of lung epithelial cells (Ahn et al., J Virol., 2019, 93(20): e00662-19).

The nucleotide sequence of the genomic region of human chromosome harboring the Testican-2 gene (SPOCK2) may be found in, for example, the Genome Reference Consortium Human Build 38 available at GenBank. The nucleotide sequence of the genomic region of human chromosome 10 harboring the Testican-2 gene may also be found at, for example, GenBank Accession No. NC_000010.11, corresponding to nucleotides 72059034-72095313 of human chromosome 10. Exemplary nucleotide and amino acid sequences of Testican-2 can be found, for example, at GenBank Accession No. NM_001244950.2 (Homo sapiens SPARC/osteonectin, cwcv and kazal like domains proteoglycan 2 (SPOCK2), transcript variant 3). Amino acid sequence of human Testican-2 (isoform 2 precursor) is provided below:

(SEQ ID NO: 11)
MRAPGCGRLVLPLLLLAAAALAEGDAKGLKEGETPGNFMEDEQWLSS
ISQYSGKIKHWNRFRDEVEDDYIKSWEDNQQGDEALDTTKDPCQKVK
CSRHKVCIAQGYQRAMCISRKKLEHRIKQPTVKLHGNKDSICKPCHM
AQLASVCGSDGHTYSSVCKLEQQACLSSKQLAVRCEGPCPCPTEQAA
TSTADGKPETCTGQDLADLGDRLRDWFQLLHENSKQNGSASSVAGPA
SGLDKSLGASCKDSIGWMFSKLDTSADLFLDQTELAAINLDKYEVCI
RPFFNSCDTYKDGRVSTAEWCFCFWREKPPCLAELERIQIQEAAKKK
PGIFIPSCDEDGYYRKMQCDQSSGDCWCVDQLGLELTGTRTHGSPDC
DDIVGFSGDFGSGVGWEDEEEKETEEAGEEAEEEEGEAGEADDGGYI
W

Testican-2 sequences can also be found in publicly available databases, for example, GenBank, OMIM, and UniProt (Q92563). Additional information on Testican-2 (SPOCK2) can be found, for example, at the NCBI web site that refers to gene 9806. The term Testican-2 as used herein also refers to variations of the SPOCK2 gene including variants provided in the clinical variant database, for example, at the NCBI clinical variants web site that refers to the term NM_001244950.2.

The entire contents of each of the foregoing GenBank Accession numbers and the Gene database numbers are incorporated herein by reference as of the date of filing this application.

### III. Methods and Compositions for Determining Risk of RD and ESRD Based on Protective Proteins

The instant disclosure is based, at least in part, on the discovery that levels of certain protective proteins can be used to identify a human subject who is at risk of progressive kidney disease or progressing to end-stage kidney disease. The low level of a protective protein identified herein, relative to a person who does not have progressive kidney failure, indicates who will be protected from progressing to end-stage kidney disease and who will not. Another embodiment described herein is the treatment of a human patient identified as being at risk for ESKD, where, e.g., administration of the protective protein, or a combination thereof, decreases the risk of the patient from progressive kidney disease.

Examples of protective proteins that may be used in the methods and compositions as described herein are provided herein. As described herein, the term protective proteins is intended to include the protective proteins, as well as functional fragments thereof. A functional fragment would retain, for example, the ability ascribed to corresponding full length (or non-fragment) equivalent.

The expression level of one or more protective proteins may be determined in a biological sample derived from a subject. A sample derived from a subject is one that originates and is obtained from a subject. Such a sample may be further processed after it is obtained from the subject. For example, protein may be isolated from a sample. In one embodiment, the protein isolated from the sample is also a sample derived from a subject. A biological sample useful for determining the level of one or more protective protein may be obtained from essentially any source, as protein expression has been reported in cells, tissues, and fluids throughout the body. However, in one aspect of the disclosure, levels of one or more protective proteins indicative of a subject having renal decline and/or ESKD, or a risk of having renal decline and/or developing ESKD, may be detected in a sample obtained from a subject non-invasively.

In certain embodiments, the biological sample used for determining the level of one or more protective proteins is a sample containing circulating protein biomarkers. Extracellular protein biomarkers freely circulate in a wide range of biological material, including bodily fluids, such as fluids from the circulatory system, e.g., a blood sample or a lymph sample, or from another bodily fluid such as cerebrospinal fluid (CSF), urine or saliva. Accordingly, in some embodiments, the biological sample used for determining the level of one or more protective proteins is a bodily fluid, for example, blood, fractions thereof, serum, plasma, urine, saliva, tears, sweat, semen, vaginal secretions, lymph, bronchial secretions, CSF, etc. In some embodiments, the sample is a sample that is obtained non-invasively. In one particular embodiment, the sample is a urine sample. In another embodiment, the sample is a plasma sample. In another embodiment, the sample is a serum sample.

In some embodiments, the biological sample used for determining the level of one or more protective proteins may contain cells. In other embodiments, the biological sample may be free or substantially free of cells (e.g., a serum sample). In some embodiments, a sample containing circulating protein biomarkers, is a blood-derived sample. Exemplary blood-derived sample types include, e.g., a blood sample, a plasma sample, a serum sample, etc. In other embodiments, a sample containing circulating protein biomarkers is a lymph sample. Circulating protein biomarkers are also found in urine and saliva, and biological samples derived from these sources are likewise suitable for determining the level of one or more protective proteins.

### Compositions for Determining Protective Protein Levels

Also disclosed herein are arrays (e.g., protein arrays) or compositions comprising antibodies, or antigen-binding fragments thereof, specific for any one or more of FGF20, TNFSF12, ANGPT1, SPARC, CCL5, APP, PF4, DNAJC19, and Testican-2, for performing the methods described herein. Such arrays may include a support or a substrate for attaching any one or more of the antibodies, or antigen-binding fragments thereof, specific for any one or more of FGF20, TNFSF12, ANGPT1, SPARC, CCL5, APP, PF4, DNAJC19, and Testican-2. Such supports and substrates are known in the art and include covalent and noncovalent interactions. For example, diffusion of applied proteins (e.g., antibodies, or antigen-binding fragments thereof) into a porous surface such a hydrogel allows noncovalent binding of unmodified protein within hydrogel structures. Covalent coupling methods provide a stable linkage and may be applied to a range of proteins. Biological capture methods utilizing a tag (e.g., hexahistidine (SEQ ID NO: 10)/Ni-NTA or biotin/avidin) on a protein (e.g., a biomarker) and a partner reagent immobilized on the surface of the substrate provide a stable linkage and bind the protein (e.g., a biomarker) specifically and in reproducible orientation.

In one embodiment, the antibodies, or antigen-binding fragments thereof, specific for any one or more of FGF20, TNFSF12, ANGPT1, SPARC, CCL5, APP, PF4, DNAJC19, and Testican-2 described herein are coated or spotted onto the support or substrate such as chemically derivatized glass, or a glass plate coated with a protein binding agent such as, but not limited to, nitrocellulose.

In another embodiment the antibodies, or antigen-binding fragments thereof, specific for any one or more of FGF20, TNFSF12, ANGPT1, SPARC, CCL5, APP, PF4, DNAJC19, and Testican-2 are provided in the form of an array, such as a microarray. Protein microarrays are known in the art and reviewed for example by Hall et al. (2007) Mech Ageing Dev 128:161-167 and Stoevesandt et al (2009) Expert Rev Proteomics 6:145-157, the disclosures of which are incorporated herein by reference. Microarrays may be prepared by immobilizing purified antigens on a substrate such as a treated microscope slide using a contact spotter or a non-contact microarrayer. Microarrays may also be produced through in situ cell-free synthesis directly from corresponding DNA arrays. A microarray may be included in test panels for performing methods disclosed herein. The production of the microarrays is in certain circumstances performed with commercially available printing buffers designed to maintain the three-dimensional shape of the antigens. In one embodiment, the substrate for the microarray is a nitrocellulose-coated glass slide.

The assays are performed by methods known in the art in which the one or more antibodies, or antigen-binding fragments thereof, specific for any one or more of FGF20, TNFSF12, ANGPT1, SPARC, CCL5, APP, PF4, DNAJC19, and Testican-2 are contacted with a biological sample under conditions that allow the formation of an immunocomplex of an antibody and any one or more of FGF20, TNFSF12, ANGPT1, SPARC, CCL5, APP, PF4, DNAJC19, and Testican-2 for detecting the immunocomplex. The presence and amount of the immunocomplex may be detected by methods known in the art, including label-based and label-free detection. For example, label-based detection methods include addition of a secondary antibody that is coupled to an indicator reagent comprising a signal generating compound. The secondary antibody may be an anti-human IgG antibody. Indicator reagents include chromogenic agents, catalysts such as enzyme conjugates, fluorescent compounds such as fluorescein and rhodamine, chemiluminescent compounds such as dioxetanes, acridiniums, phenanthridiniums, ruthenium, and luminol, radioactive elements, direct visual labels, as well as cofactors, inhibitors and magnetic particles. Examples of enzyme conjugates include alkaline phosphatase, horseradish peroxidase and beta-galactosidase. Methods of label-free detection include surface plasmon resonance, carbon nanotubes and nanowires, and interferometry. Label-based and label-free detection methods are known in the art and disclosed, for example, by Hall et al. (2007) and by Ray et al. (2010) Proteomics 10:731-748. Detection may be accomplished by scanning methods known in the art and appropriate for the label used, and associated analytical software.

As described herein, protective proteins indicative of renal decline and/or ESKD and/or protective proteins indicative of an increased risk of renal decline and/or an increased risk of progression to ESKD are disclosed. It is thus contemplated that protective proteins levels can be assayed from a sample from a subject, such as a test subject (e.g., a subject who is suspected of having renal decline and/or ESKD, or a subject who is at increased risk of having renal decline and/or ESKD) in order to determine whether the test subject has renal decline and/or ESKD, or whether the test subject is at an increased risk of renal decline and/or an increased risk of progression to ESKD. In certain embodiments, protective proteins were identified by comparing the levels of certain proteins (e.g., circulating proteins) in, for example, samples from subjects who developed renal decline and/or ESKD, or in samples from subjects with diabetes (T1D, T2D) who were at risk for renal decline and rapid progression to ESKD, and compared to levels of certain proteins (e.g., circulating proteins) in, for example, samples from subjects who did not develop renal decline and/or ESKD, or in samples from subjects with diabetes (T1D, T2D) who were determined to have stable kidney function (i.e., were non-progressors), or in samples from healthy control subjects, or in samples of a standard control level or reference level. In other embodiments, protective proteins were identified by comparing the levels of certain proteins (e.g., circulating proteins) in, for example, samples from subjects who developed renal decline and/or ESKD, or in samples from subjects with diabetes (T1D, T2D) who were at risk for renal decline and rapid progression to ESKD, and compared to known baseline concentration of proteins (e.g., circulating proteins or plasma proteins), known or measured, for example, by a proteomics platform (e.g., SOMAscan platform, and/or OLINK platform). A number of differentially present protein biomarkers were identified in this manner, and were determined to be indicative of a subject having renal decline and/or ESKD, at indicative of an increased risk of renal decline and/or progression to ESKD, which include, but are not limited to, FGF20, TNFSF12, ANGPT1, SPARC, CCL5, APP, PF4, DNAJC19, and/or Testican-2.

The protective proteins identified herein can be used to determine whether a subject, for example a subject with T1D or T2D, has or is at risk of developing renal decline and/or ESKD, and whose risk of developing renal decline and/or ESKD was previously unknown. This may be accomplished by determining the level of one or more of FGF20, TNFSF12, ANGPT1, SPARC, CCL5, APP, PF4, DNAJC19, and/or Testican-2, or combinations thereof, in a biological sample derived from the subject. A difference in the level of one or more of these protective proteins as compared to that in a biological sample derived from a normal subject (i.e., a subject known to not have renal decline and/or ESKD; or a normoalbuminuric control level, or a healthy control level, or a standard control level) may be predictive regarding whether the subject has a risk of developing renal decline and/or ESKD.

The level of one or more protective proteins in a biological sample may be determined by any suitable method. Any reliable method for measuring or detecting the level or amount of protein in a sample may be used. Accordingly, practicing the methods disclosed herein may utilize routine techniques in the field of molecular biology. Basic texts disclosing the general methods of use in this disclosure include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

The present disclosure relates to a method (e.g., in vitro method) of measuring or detecting the amount of certain protein levels found in a cell, tissue, or sample (e.g., a plasma sample or a serum sample) of a subject, as a means to detect the presence, to assess the risk of developing, diagnosing, prognosing, and/or monitoring the progression of and/or monitoring the efficacy of a treatment for renal decline and/or ESKD. Thus, in certain embodiments, the first steps of practicing the methods of this disclosure (e.g., in vitro methods of using certain identified biomarkers for diagnosis, prognosis, and/or monitoring of renal decline and/or ESKD) are to obtain a cell, tissue or sample (e.g. a urine sample or a plasma sample or a serum sample) from a test subject and extract protein from the sample.

Samples may be prepared according to methods known in the art. Cell, tissue or blood samples (e.g., a plasma sample or a serum sample) from a subject is suitable for the present disclosure and may be obtained using well-known methods and as described herein. In certain embodiments of the disclosure, a plasma sample is a preferred sample type. In other embodiments of the disclosure, a serum sample is a preferred sample type.

In some embodiments, a biological sample (e.g., a cell, a tissue, a plasma sample or a serum sample) is obtained from a subject to be tested or monitored for renal decline and/or ESKD as described herein. Biological samples of the same type should be taken from both a test subject (e.g., a subject suspected to have renal decline and/or ESKD and/or a subject at a risk of developing renal decline and/or ESKD) and a control subject (e.g., a subject not suffering from renal decline and/or ESKD; e.g., a sample from a normoalbuminuric control subject, or from a healthy control subject, or of a known/standard control level)). Collection of a biological sample from a subject, such as a test subject, may be performed in accordance with the standard protocol hospitals or clinics generally follow. An appropriate amount of biological sample (e.g., a cell, a tissue or plasma sample) is collected and may be stored according to standard procedures prior to further preparation.

The analysis of certain protective proteins, as described herein, found in a biological sample of a subject (e.g., test subject) according to the method disclosed herein may be performed in certain embodiments, using, e.g., a cell, a tissue, a urine sample, a plasma sample or a serum sample. The methods for preparing biological samples for protein extraction are well known among those of skill in the art. For example, a cell population or a tissue sample of a subject (e.g., test subject) should be first treated to disrupt cellular membrane so as to release protein contained within the cells.

For the purpose of detecting the presence of certain protective proteins disclosed herein or assessing whether a test subject has or is at risk of developing renal decline and/or ESKD, a biological sample may be collected from the subject and the level of certain protective proteins disclosed herein may be measured and then compared to the normal level of these same certain protective proteins (e.g., compared to the level of the certain protective proteins disclosed herein in same type of biological sample in the subject before the onset of renal decline and/or ESKD, and/or compared to the level of the certain protective proteins disclosed herein in same type of biological sample from a healthy control subject (e.g., a subject who does not have T1D or T2D), and/or compared to a known control standard of baseline levels of the certain protective proteins disclosed herein). If a level of one or more certain protective proteins disclosed herein is statistically significantly lower when compared to the normal level of the one or more certain protective proteins disclosed herein, the test subject is deemed to have renal decline and/or ESKD or have an increased risk of developing renal decline and/or ESKD. For the purpose of monitoring disease progression or assessing therapeutic effectiveness in renal decline and/or ESKD patients, a biological sample from a test subject may be taken at different time points, such that the level of the certain protective proteins disclosed herein can be measured over time (i.e., serial testing) to provide information indicating the state of disease. For instance, when the level of the certain protective proteins disclosed herein from a test subject shows a general trend of increasing or stabilizing to a normal level over time, the test subject is deemed to be improving or stabilizing in the severity of renal decline and/or ESRD or the therapy the patient has been receiving is deemed effective. A lack of an increase or stabilization in the level of the certain protective proteins disclosed herein from a test subject or a continuing trend of decreasing levels of the certain protective proteins disclosed herein from a test subject would indicate a worsening of the condition and ineffectiveness of the therapy given to the patient. Generally, a comparatively lower level of the certain protective proteins disclosed herein seen in a test subject indicates that the test subject has renal decline and/or ESKD and/or that the test subject's renal decline and/or ESKD condition is worsening or that renal decline and/or ESKD is progressing.

A protein of any particular identity, such as a protective protein(s) as disclosed herein, can be detected using a variety of immunological assays. In some embodiments, a sandwich assay can be performed by capturing the protective protein(s) from a test sample with an antibody (or antibodies) having specific binding affinity for the protective protein(s). The protective protein(s) can subsequently be detected using, e.g., a labeled antibody having specific binding affinity for the protective protein(s). One common method of detection is the use of autoradiography by using a radiolabeled detection agent (e.g., a radiolabeled anti-protective protein specific antibody) that is labeled with radioisotopes (e.g., ³H, ¹²⁵I, ³⁵S, ¹⁴C, or ³²P, ^{99m}Tc, or the like). The choice of radioactive isotope depends on research preferences due to ease of synthesis, stability, and half-lives of the selected isotopes. Other labels that can be used for labeling of detection agents (e.g., for labeling of anti-biomarker specific antibody) include compounds (e.g., biotin and digoxigenin), which bind to anti-ligands or antibodies labeled with fluorophores, chemiluminescent agents, fluorophores, and enzymes (e.g., HRP). Such immunological assays can be carried out using microfluidic devices such as microarray protein chips. A protein of interest (e.g., a protective protein(s) as disclosed herein) can also be detected by gel electrophoresis (such as 2-dimensional gel electrophoresis) and western blot analysis using specific antibodies (e.g., anti-protective proteins specific antibodies). In some embodiments, standard ELISA techniques can be used to detect a given protein (e.g., a protective protein as disclosed herein), using an appropriate antibody (or antibodies), e.g., an anti-protective protein specific antibody. In other embodiments, standard western blot analysis techniques can be used to detect a given protein (e.g., a protective protein as disclosed herein), using the appropriate antibodies. Alternatively, standard immunohistochemical (IHC) techniques can be used to detect a given protective protein, using an appropriate antibody (or antibodies), e.g., an anti-protective protein specific antibody. Both monoclonal and polyclonal antibodies (including an antibody fragment with desired binding specificity) can be used for specific detection of the protective protein(s). Such antibodies and their binding fragments with specific binding affinity to a particular protein (e.g., a protective protein(s) as disclosed herein) can be generated by known techniques.

In some embodiments, a protective protein as disclosed herein can be detected (e.g., can be detected in a detection assay) with an antibody that binds to the protective protein, such as an anti-protective protein specific antibody, or an antigen-binding fragment thereof. In certain embodiments, an anti-protective protein specific antibody is used as a detection agent, such as a detection antibody that binds to a protective protein(s) as disclosed herein and detects the protective protein(s) (e.g., from a biological sample), such as detects the protective protein(s) in a detection assay (e.g., in western blot analysis, immunohistochemistry analysis, autoradiography analysis, and/or ELISA). In certain embodiments, an anti-protective protein specific antibody is used as a capture agent that binds to the protective protein and detects the protective protein (e.g., from a biological sample), such as detects the protective protein in a detection assay (e.g., in western blot analysis, immunohistochemistry analysis, autoradiography analysis, and/or ELISA). In some embodiments, an anti-protective protein specific antibody, or an antigen-binding fragment thereof is labeled for ease of detection. In some embodiments, anti-protective protein specific antibody, or an antigen-binding fragment thereof, is radiolabeled (e.g., labeled with a radioisotope, such as labeled with ³H, ¹²⁵I, ³⁵S, ¹⁴C, or ³²P, ^{99m}Tc, or the like), enzymatically labelled (e.g., labeled with an enzyme, such as with horseradish peroxidase (HRP)), fluorescent labeled (e.g., labeled with a fluorophore), labeled with a chemiluminescent agent and/or labeled with a compound (e.g., with biotin and digoxigenin).

Other methods may also be employed for measuring or detecting the level of protective proteins as disclosed herein in practicing the present disclosure. For instance, a variety of methods have been developed based on the mass spectrometry technology to rapidly and accurately quantify target proteins even in a large number of samples. These methods involve highly sophisticated equipment such as the triple quadrupole (triple Q) instrument using the multiple reaction monitoring (MRM) technique, matrix assisted laser desorption/ionization time-of-flight tandem mass spectrometer (MALDI TOF/TOF), an ion trap instrument using selective ion monitoring SIM) mode, and the electrospray ionization (ESI) based QTOP mass spectrometer. See, e.g., Pan et al., J Proteome Res 2009 February; 8(2):787-797.

In other embodiments, the expression of a protective protein as disclosed herein is evaluated by assessing the protective protein as disclosed herein. In some embodiments, an anti-protective protein specific antibody, or fragment thereof, can be used to assess the protective protein. Such methods may involve using IHC, western blot analyses, ELISA, immunoprecipitation, autoradiography, or an antibody array. In particular embodiments, the protective protein is assessed using IHC. The use of IHC may allow for quantitation and characterization of the protective protein. IHC may also allow an immunoreactive score for the sample in which the expression of the protective protein is to be determined. The term “immunoreactive score” (IRS) refers to a number that is calculated based on a scale reflecting the percentage of positive cells (on a scale of 1-4, where 0=0%, 1=<10%, 2=10%-50%, 3=50%-80%, and 4=>80%) multiplied by the intensity of staining (on a scale of 1-3, where 1=weak, 2=moderate, and 3=strong). IRS may range from 0-12.

In certain other embodiments, the SOMAscan—Aptamer-based proteomic platform may be used to determine levels of the protective proteins as disclosed herein. This platform technology is based on the recognition that unique single-stranded sequences of DNA and RNA, referred to as aptamers, are capable of recognizing folded protein epitopes with high affinity and specificity. This property was further advanced with the use of the SOMAscan platform to assay concentrations of proteins (uses one aptamer per protein). This platform features high throughput capabilities (over 1000 proteins in one sample), with reproducibility and sensitivity.

In certain other embodiments, the OLINK-Proximity Extension Assay based proteomic platform may be used to determine levels of the protective protein(s) as disclosed herein. The OLINK Proximity Extension Assay is a molecular technique that merges an antibody-based immunoassay with the powerful properties of PCR and quantitative real-time PCR (qPCR), resulting in a multi-plexable and highly specific method (e.g., uses two antibodies per protein) numerous protective proteins can be quantified simultaneously using only 1 μL of plasma/serum. These assays were thoroughly validated and grouped as panels designed to focus on specific diseases or biological processes and were optimized for the expected dynamic range of the target protein concentrations in clinical samples.

As described herein, the estimated Glomerular Filtration Rate (eGFR) refers to a means for estimating kidney function. In some embodiments, the method described herein comprises measuring an estimated glomerular function rate (eGFR) slope of the human subject and determining whether the eGFR slope of the human subject indicates that the human subject has or is at risk of developing renal decline. In some embodiments, eGFR is determined based on a measurement of serum creatinine levels. In other embodiments, eGFR is determined based on a measurement of serum cystatin C levels. In other embodiments, eGFR is determined using ordinary least squares assuming linear regression with at least 3 serum creatinine values available and measured at least 6 months apart. In other embodiments, eGFR is determined using ordinary least squares assuming linear regression with at least 3 serum creatinine values available and measured at least 1 year apart. In yet other embodiments, eGFR is determined using ordinary least squares assuming linear regression with at least 3 serum creatinine values available and measured at least 2 or more years apart. In other embodiments, eGFR is estimated by visual inspection.

In some embodiments, an eGFR slope of at least <−3 ml/min/year (i.e., eGFR loss ≥3.0 ml/min/year) indicates that the human subject has or is at risk of developing renal decline. In other embodiments, an eGFR slope of at least <−5 ml/min/year indicates that the human subject has or is at risk of developing renal decline. In yet other embodiments, an eGFR slope of at least <−10 ml/min/year indicates that the human subject has or is at risk of developing renal decline. In yet another embodiment, an eGFR slope of at least <−15 ml/min/year indicates that the human subject has or is at risk of developing renal decline. In other embodiments, a ≥40% sustained decline in eGFR from baseline (confirmed for at least 3 months) indicates that the human subject has or is at risk of developing renal decline.

In yet another embodiment, eGFR may be determined using the CKD-EPI creatinine equation. In some embodiments, the estimation of GFR slopes may depend on the subject's race, sex and serum creatinine levels. For example, in one embodiment, the eGFR slope for a female of African descent with a serum creatinine concentration (μmol/dL) of ≤62 (≤0.7) is determined using the following expression: GFR=166×(Scr/0.7)^{-0.329}×(0.993)^{Age}. In another embodiment, the eGFR slope for a female of African descent with a serum creatinine concentration (μmol/dL) of >62 (>0.7) is determined using the following expression: GFR=166×(Scr/0.7)^{-1.209}×(0.993)^{Age}. In another embodiment, the eGFR slope for a male of African descent with a serum creatinine concentration (μmol/dL) of ≤80(≤0.9) is determined using the following expression: GFR=163×(Scr/0.9)^{-0.411}×(0.993)^{Age}. In another embodiment, the eGFR slope for a male of African descent with a serum creatinine concentration (μmol/dL) of >80 (>0.9) is determined using the following expression: GFR=163×(Scr/0.9)^{-1.209}×(0.993)^{Age}. In another embodiment, the eGFR slope for a female of non-African decent with a serum creatinine concentration (μmol/dL) of ≤62 (≤0.7) is determined using the following expression: GFR=144×(Scr/0.7)^{-0.329}×(0.993)^{Age}. In another embodiment, the eGFR slope for a female of non-African decent with a serum creatinine concentration (μmol/dL) of >62 (>0.7) is determined using the following expression: GFR=144×(Scr/0.7)^{-1.209}×(0.993)^{Age}. In another embodiment, the eGFR slope for a male of non-African decent with a serum creatinine concentration (μmol/dL) of ≤80 (≤0.9) is determined using the following expression: GFR=141×(Scr/0.9)⁻⁰⁴¹¹×(0.993)^{Age}. In another embodiment, the eGFR slope for a male of African descent with a serum creatinine concentration (μmol/dL) of >80 (>0.9) is determined using the following expression: GFR=141×(Scr/0.9)^{-1.209}×(0.993)^{Age}.

Additional methods for determining an estimated Glomerular Filtration Rate are known among those of skill in the art.

A method described herein may further comprise combining electronic health records (EHR) and biomarkers (e.g., one or more of SPARC, CCL5, APP, PF4, DNAJC19, ANGPT1, TNFSF12, FGF20, and Testican-2) by using a machine-learned, prognostic risk-score assay as an in vitro diagnostic for enabling accurate risk prediction of progressive kidney decline.

In some embodiments, the machine-learned, prognostic risk-score assay is KIDNEYINTELX™. To this end, a random forest model can be trained, and performance (e.g., area under the curve (AUC), positive and negative predictive values (PPV/NPV), and net reclassification index (NRI)) can be compared to a clinical model and KDIGO categories for predicting a composite outcome of estimated glomerular filtration rate (eGFR) decline of ≥5 ml/min/year, ≥40% sustained decline, or kidney failure within 5 years. In some embodiments, an observational cohort study of patients with prevalent diabetic kidney disease (DKD)/banked plasma from two HER-linked biobanks can be used. KIDNEYINTELX™ can provide improved prediction of kidney outcomes over KDIGO (Kidney Disease: Improving Global Outcomes) guidelines and clinical models in individuals with early stages of DKD. In some embodiments, a machine learning model, as described in PCT Application No. PCT/US2021/018030 (publication no. WO/2021/163619; the methods and compositions of which are incorporated by reference herein) is used in the methods described herein.

The 8 protective protein biomarkers can be measured in a proprietary, analytically validated multiplex format using the Mesoscale platform (MesoScale Diagnostics, Gaithersburg, Maryland, USA), which employs electrochemiluminescence detection methods combined with patterned arrays to allow for multiplexing of assays. Each sample can be run in duplicate, along with quality control samples with known low, moderate, and high concentrations of each biomarker on each plate. Assay precision can be assessed using a panel of reference samples that span the measurement range. Levey-Jennings plots can be employed and Westguard rules can be followed the for re-run of samples. The laboratory personnel performing the biomarker assays may be blinded to all clinical information.

eGFR can be determined using the CKD-EPI creatinine equation, as described, for example, in Levey et al. (Ann Intern Med 150(9): 604-61221 (2009)). Linear mixed models can be employed with an unstructured variance-covariance matrix and random intercept/slope can be used for each individual to estimate eGFR slope, as described, for example, in Leffondre et al. (Nephrol Dial Transplant 30(8): 1237-1243 (2015)). The primary composite outcome, progressive decline in kidney function, can include the following: RKFD defined as an eGFR slope decline of ≥5 ml/min/1.73 m²/year; a sustained (confirmed at least 3 months later) decline in eGFR of ≥40% from baseline; or “kidney failure” defined by sustained eGFR<15 ml/min/1.73 m² confirmed at least 30 days later; or receipt of long-term maintenance dialysis or receipt of a kidney transplant (KDIGO, Kidney Int Suppl 3: 1-163 (2012); Levey et al. Am J Kidney Dis 64(6): 821-835(2014)). Additionally, nephrologists (SC/GNN) can be employed to independently adjudicate all outcomes, examine each individual patient over their longitudinal course, and account for eGFR changes (ensuring annualized decline of ≥5 ml/min or ≥40% sustained decrease), corresponding ICD/CPT codes and medications to ensure that outcomes represented true decline rather than a context dependent temporary change (e.g., due to medications/hospitalizations). Follow up time can be censored after loss to follow-up, after the date that the non-slope components of the composite kidney endpoint are met, or 5 years after baseline.

The datasets can be randomized into a derivation (60%) and validation sets (40%). The validation dataset can be completely blinded and sequestered from the total derivation dataset. Using only the derivation set, supervised random forest algorithms on the combined biomarker and all structured EHR features can be evaluated without a priori feature selection and a candidate feature set can be identified. The derivation set can then be randomly split into secondary training and test sets for model optimization with 70%-30% spitting and a 10-fold cross-validation for AUC. Both raw values and ratios of the biomarkers can be considered. Missing uACR values can be imputed to 10 mg/g (Nelson et al. JAMA (2019)), missing blood pressure (BP) values can be imputed using multiple predictors (age, sex, race and antihypertensive medications) (De Silva et al. BMC Med Res Methodol 17(1): 114 (2017)) and median value can be used for other features where missingness was <30%.

Further iterations of the model can be conducted by tuning the individual hyperparameters. A hyperparameter is a parameter which is used to control the learning process (e.g., number of RF trees) as opposed to parameters whose weights are learned during the training (e.g., weight of a variable). Tuning hyperparameters refers to iteration of model architecture after setting parameter weights to achieve the ideal performance. Hyperparameters optimization can be performed using grid search approach. K-fold cross validation based AUC can be evaluated for all possible combinations of hyperparameters. Combination of hyperparameters which optimize the AUC for model building can be selected. The following hyperparameters can be considered for optimization: number of variables randomly selected as candidates for splitting a node; forest average number of unique cases (data points) in a terminal node; maximum depth to which a tree should be grown.

Additionally, the code for hyperparameter optimization can be deposited in a github repository (https://github.com/girish-nadkarni/KidneyIntelX_hyperparameter_tuning) for improving reproducibility and transparency. The final model can be selected based on AUC performance.

Risk probabilities for the composite kidney endpoint can be generated using the final model in the derivation set, scaled to align with a continuous score from 5-100 by increments of 5, and this score can be applied to the validation set. Risk cut-offs can be chosen in the derivation set to encompass the top 15% as the high risk (scores 90-100), bottom 45% as the low risk (scores 5-45), and the intervening 40% as the intermediate risk group (scores 50-85). Primary performance criteria can be AUC, positive predictive value for high risk group and negative predictive values for low risk group (PPV and NPV, respectively) at the pre-determined cut-offs. The selected model and associated cut-offs can then be validated by an independent biostatistician (MK) in the sequestered validation cohort.

In addition to these traditional test statistics, calibration can be assessed by examination of the slope of observed vs. expected outcome plots of the KIDNEYINTELX score vs. only the observed outcomes. Also, Kaplan Meier curves can be constructed for time-dependent outcomes of 40% decline and kidney failure with hazard ratios using the Cox proportional hazards method. The discrimination of the KIDNEYINTELX model can be compared to a recently validated comprehensive clinical model which includes age, sex, race, eGFR, cardiovascular disease, smoking, hypertension, BMI, UACR, insulin, diabetes medications, and HbA1c and is developed to predict 40% eGFR decline in eGFR in T2D (Nelson et al. JAMA (2019)). Utility metrics (PPV, NPV) can be compared to both the comprehensive clinical model and KDIGO risk strata.

Finally, the net reclassification index (NRI) for events and non-events compared to KDIGO risk strata can be calculated (Pencina et al. Stat Med 27(2): 157-172 (2008); Pencina et al. Stat Med 30(1): 11-21 (2010)). All a-priori levels of significance can be <0.05. All hypothesis tests can be two-sided. 95% confidence intervals can be calculated by bootstrapping. All analyses can be performed with R software (www.rproject.org), the dplyr package, the randomForestSRC, and the CARET package (Hadley et al. (2020) dplyr: A Grammar of Data Manipulation. R Package version 0.7.6. Available from cran.r-project.org/web/packages/dplyr/index.html); Hemant Ishwaran UBK (2020) randomForestSRC: Fast Unified Random Forests for Survival, Regression, and Classification (RF-SRC). Available from cran.rproject.org/web/packages/randomForestSRC/index).

Utilizing patients with T2D from two biobanks with plasma samples and linked EHR data, a risk score can be developed and validated combining clinical data and plasma biomarkers via a random forest algorithm to predict a composite kidney outcome, progressive decline in kidney function, consisting of RKFD, sustained 40% decline in eGFR, and kidney failure over 5 years. KIDNEYINTELX can be demonstrated to outperform models using only standard clinical variables, including KDIGO risk categories (KDIGO, Kidney Int Suppl 3: 1-163 (2012)). Marked improvements can be seen in discrimination over clinical models, as measured by AUC, NRI, and improvements in PPV compared to KDIGO risk categories. Furthermore, KIDNEYINTELX can accurately identify over 40% more patients experiencing events than the KDIGO risk strata. Finally, KIDNEYINTELX can provide good risk stratification for the accepted FDA endpoint of sustained 40% decline in eGFR or kidney failure with a 15-fold difference in risk between the high-risk and low-risk strata for this clinical and objective endpoint.

DKD is an increasingly complex and common problem challenging modern healthcare systems. In real world practice, the prediction of DKD progression is challenging, particularly in early disease with preserved kidney function and therefore, implementation of improved prognostic tests is paramount. Integrated risk score has near-term clinical implications, especially when linked to clinical decision support (CDS) and embedded care pathways. The current standard for clinical risk stratification (KDIGO risk strata) (KDIGO KDIGO, Kidney Int Suppl 3: 1-163 (2012)) has three risk strata that overlap with the population of DKD patients that can be included in the KIDNEYINTELX study. A risk score with three risk strata (low, intermediate, and high) can be created by incorporating KDIGO classification components (eGFR and uACR), as well as the addition of other clinical variables, and three blood-based biomarkers. In this way, the ability to accurately risk-stratify patients with DKD can be augmented, thereby enabling improved patient management.

Care for low-risk patients with DKD can be continued with their existing PCP's or diabetologists and require less intensity treatments, unless repeat testing, changes in clinical status or local arrangements regarding referral to specialist care indicate otherwise. For those with high-risk scores, oversight may include more referrals to nephrology (Smart et al. The Cochrane database of systematic reviews (6): CD007333 (2014); Smart and Titus, Am J Med 124(11): 1073-1080 e1072 (2011)), increased monitoring intervals, improved awareness of kidney health, referral to dieticians, reinforcement of usage of antagonists of the renin angiotensin aldosterone system, and increased motivation to start recently approved medications, including SGLT2 inhibitors and GLP-1 receptor agonists to slow progression (Kristensen et al. Lancet Diabetes Endocrinol 7(10): 776-785 (2019); Sarafidis et al. Nephrol Dial Transplant 34(2): 208-230 (2019)). Adoption of these new therapies is lagging, especially in patients considered to be ‘lowrisk’ by standard criteria, where cost of treatment and presence of adverse events are limiting factors. Earlier engagement with nephrologists may also allow for more time to advise and educate patients about homebased dialysis and pre-emptive or early kidney transplant as patient-centered kidney replacement options if more aggressive treatment does not ultimately prevent progression of DKD. The use of a risk score as part of the enrollment process in future RCTs may enrich the trial participants for greater likelihood of events and thus reduce the chances for type 2 error, or minimize the sample size needed to detect a statistically significant difference with treatment vs. control. Interventions that prevent or slow DKD progression and foster patient-centered kidney replacement modalities support the goals of the US Department of Health and Human Services' Advancing American Kidney Health initiative (Mehrotra, Clin J Am Soc Nephrol 14(12): 1788 (2019)).

KIDNEYINTELX included inputs from biomarkers examined in several settings, including patients with DKD. Soluble TNFR1 and 2 and plasma KIM-1 have demonstrated reliable independent prognostic signals for kidney function decline and ESKD (Niewczas et al. J Am Soc Nephrol 23(3): 507-515 (2012); Coca et al. J Am Soc Nephrol 28(9): 2786-2793 (2017); Nadkarni et al. Kidney Int 93(6): 1409-1416 (2018); Tummalapalli et al. Curr Opin Nephrol Hypertens 25(6): 480-486 (2016); Gohda et al. J Am Soc Nephrol 23(3): 516-524 (2012); Krolewski et al. Diabetes care 37(1): 226-234 (2014); Bhatraju et al. J Am Soc Nephrol 29(11): 2713-2721 (2018)). In a previous study, it was found that inclusion of biomarkers to clinical data derived from EHR at a single-center had better predictive performance than clinical models alone (Chauhan et al. Kidney360 (2020)). However, that study included few patients with prevalent CKD (approximately ⅓rd had CKD in the cohort with T2D and ¼th had CKD in the APOL1 high-risk cohort). However, in the method described hereinabove, by incorporating biomarker concentrations and the EHR data into the machine learning algorithm, a multidimensional representation of risk for patient with DKD can be provided and improved prognostic estimates for future progression can be generated (Tangri et al. JAMA 315(2): 164-174 (2016); Tangri et al. JAMA 305(15): 1553-1559 (2011)). Other composite tests that incorporate multiple plasma biomarkers and limited clinical data features have been shown to accurately predicted incident CKD in individuals with T2D, although prediction of progressive decline in kidney function is an ongoing challenge (Peters et al. J Clin Med 9(10) (2020); Peters et al. J Diabetes Complicat 33(12) (2019)). However, the goal of KIDNEYINTELX test is to determine which patients with established DKD are at highest risk of progressive decline in kidney function of kidney failure and those that have CKD that is unlikely to progress over time.

Thus, a machine-learned model combining plasma biomarkers and EHR data can significantly improve prediction of progressive decline in kidney function over standard clinical models in patients with T2 DKD from large academic medical centers.

A machine-learned, prognostic risk-score assay for use with the current methods can be used, as described, for example, in U.S. Patent Application No. 62/976,767, U.S. Patent Application No. 62/976,761, and U.S. Patent Application No. 63/016,868, each of which is incorporated herein by reference in its entirety.

### IV. Methods of Treatment or Prevention

Methods and compositions for treating or preventing renal decline and/or ESKD (also referred to herein as ESRD) in a subject in need thereof are also featured in the disclosure. In one embodiment, the present disclosure provides methods of treating a subject having renal decline and/or ESKD, a subject suspected of having renal decline and/or ESKD, or a subject who is at a risk of developing renal decline and/or ESKD. In other embodiments, a subject having a disorder associated with renal decline and/or ESKD may be treated using the methods described herein without having been identified by the predictive methods of the present disclosure. In certain embodiments, methods of treatment disclosed herein improves kidney function (also referred to herein as “renal function”) in such subjects.

In some embodiments, methods of treatment described herein comprises administering to the subject a therapy of the present disclosure. A therapy of the present disclosure may comprise a therapeutically effective amount of a protein or nucleic acid molecule that increases the expression and/or function of one or more protective proteins described hereinabove. For example, a therapy of the present disclosure may comprise a therapeutically effective amount of one or more protective proteins (e.g., a therapeutically effective amount of recombinant SPARC, recombinant CCL5, recombinant APP, recombinant PF4, recombinant DNAJC19, recombinant ANGPT1, recombinant TNFSF12, recombinant FGF20, and/or recombinant Testican-2). Alternatively, a therapy of the present disclosure may comprise a therapeutically effective amount of an analog of one or more protective proteins (e.g., a therapeutically effective amount of a SPARC analog, a CCL5 analog, an APP analog, a PF4 analog, a DNAJC19 analog, an ANGPT1 analog, a TNFSF12 analog, an FGF20 analog, and/or a Testican-2 analog). An analog of a protective protein may be a mutated polypeptide (e.g., a mutated SPARC polypeptide, a mutated CCL5 polypeptide, a mutated APP polypeptide, a mutated PF4 polypeptide, a mutated DNAJC19 polypeptide, a mutated ANGPT1 polypeptide, a mutated TNFSF12 polypeptide, a mutated FGF20 polypeptide, and/or a mutated Testican-2 polypeptide). Alternatively, an analog of a protective protein may be a fusion protein, such as a chimeric protein containing the protective protein (e.g., a SPARC polypeptide, a CCL5 polypeptide, an APP polypeptide, a PF4 polypeptide, a DNAJC19 polypeptide, an ANGPT1 polypeptide, a TNFSF12 polypeptide, an FGF20 polypeptide, and/or a Testican-2 polypeptide) and one or more polypeptide portions that enhance in vivo stability, in vivo half-life, and/or uptake/administration. Alternatively, an analog of a protective protein may be a mimetic (e.g., a non-peptide mimetic) of one or more protective proteins (e.g., a mimetic of SPARC, CCL5, APP, PF4, DNAJC19, ANGPT1, TNFSF12, FGF20, and/or Testican-2). In other instances, an analog of a protective protein may be an agonist of one or more protective proteins (e.g., a SPARC agonist, a CCL5 agonist, an APP agonist, a PF4 agonist, a DNAJC19 agonist, an ANGPT1 agonist, a TNFSF12 agonist, an FGF20 agonist, and/or a Testican-2 agonist). An agonist for use in the present disclosure may be an agonistic antibody, such as an antibody directed to the receptor of the protective protein (e.g., an agnostic SPARC receptor antibody, an agnostic CCL5 receptor antibody, an agnostic APP receptor antibody, an agnostic PF4 receptor antibody, an agnostic DNAJC19 receptor antibody, an agnostic ANGPT1 receptor antibody, an agnostic TNFSF12 receptor antibody, an agnostic FGF20 receptor antibody, and/or an agnostic Testican-2 receptor antibody. In yet other embodiments, a therapy of the present disclosure may comprise a therapeutically effective amount of a nucleic acid molecule encoding one or more protein proteins (e.g., a DNA or RNA molecule encoding one or more of SPARC, CCL5, APP, PF4, DNAJC19, ANGPT1, TNFSF12, FGF20, and/or Testican-2).

### ANGPT1

In some embodiments, a method of treatment described herein comprises therapeutic use of ANGPT1, such as administering to a subject a therapeutically effective amount of a protein or nucleic acid molecule that increases the expression and/or function of ANGPT1. For example, a method of treatment described herein may comprise administering to a subject a therapeutically effective amount of recombinant ANGPT1 (e.g., of human or mouse origin), an ANGPT1 analog (e.g., a mutated ANGPT1 polypeptide, or an ANGPT1 fusion protein, such as a chimeric protein containing ANGPT1 polypeptide and one or more polypeptide portions that enhance in vivo stability, in vivo half-life, and/or uptake/administration), an ANGPT1 mimetic (e.g., a non-peptide mimetic of ANGPT1), an ANGPT1 agonist (e.g., an agonistic ANGPT1 receptor antibody) and/or a nucleic acid molecule encoding ANGPT1.

Such therapeutic use of ANGPT1 may comprise the therapeutic use, as described, for example, in WO2018067991A1. WO2018067991A1 describes a method of modulating T cell dysfunction used for treating condition e.g., cancer and chronic infection, by contacting dysfunctional T cell with a modulating agent or agents that promotes the expression, activity and/or function of an angiopoetin or angiopoietin-like protein, such as ANGPT1.

Alternatively, therapeutic use of ANGPT1 may comprise the therapeutic use, as described, for example, in US20090304680A1. US20090304680A1 describes a pharmaceutical composition for the treatment, prevention or diagnosis of Kawasaki Disease in an individual, the composition comprising a molecule comprising ANGPT1 or a modulator thereof.

### TNFSF12

In some embodiments, a method of treatment described herein comprises therapeutic use of TNFSF12 or TWEAK, such as administering to a subject a therapeutically effective amount of a protein or nucleic acid molecule that increases the expression and/or function of TNFSF12. For example, a method of treatment described herein may comprise administering to a subject a therapeutically effective amount of recombinant TNFSF12 (e.g., of human or mouse origin), a TNFSF12 analog (e.g., a mutated TNFSF12 polypeptide, or a TNFSF12 fusion protein, such as a chimeric protein containing TNFSF12 polypeptide and one or more polypeptide portions that enhance in vivo stability, in vivo half-life, and/or uptake/administration), a TNFSF12 mimetic (e.g., a non-peptide mimetic of TNFSF12), a TNFSF12 agonist (e.g., an agonistic TNFSF12 receptor antibody) and/or a nucleic acid molecule encoding TNFSF12.

Such therapeutic use of TNFSF12 may comprise the therapeutic use, as described, for example, in WO2010088534A1. As described in WO2010088534A1, TNFSF12 is capable of expanding populations of human and rodent pancreatic cells and inducing the appearance of endocrine lineage committed progenitor cells in the pancreas. Accordingly, agonists of the TNFSF12 receptor (TNFSF12-R) can be used in methods for regenerating pancreatic tissue and expanding populations of pancreatic cells in vivo and in vitro. These methods can be used to treat diseases or conditions where enhancement of pancreatic progenitor cells for cell replacement therapy is desirable, including, e.g., diabetes and conditions that result in loss of all or part of the pancreas. For use in such methods, the TNFSF12-R agonist can be TNFSF12 (e.g., TNFSF12 polypeptide of human or mouse origin), a TNFSF12 analog (e.g., a mutated TNFSF12 polypeptide, or a TNFSF12 fusion protein, such as a chimeric protein containing TNFSF12 polypeptide and one or more polypeptide portions that enhance in vivo stability, in vivo half-life, and/or uptake/administration), a TNFSF12 mimetic (e.g., a non-peptide mimetic of TNFSF12), and an agonistic TNFSF12-R antibody.

Alternatively, therapeutic use of TNFSF12 may comprise the therapeutic use, as described, for example, in WO2001085193A2. WO2001085193A2 describes use of synergistically effective amount of a TNFSF12 agonist and an angiogenic factor in a method for enhancing angiogenic activity to promote neovascularization. Such TNFSF12 agonists include soluble recombinant TNFSF12 protein and TNFSF12 agonists taught in WO98/05783, WO98/35061 and WO99/19490.

### FGF20

In some embodiments, a method of treatment described herein comprises therapeutic use of FGF20, such as administering to a subject a therapeutically effective amount of a protein or nucleic acid molecule that increases the expression and/or function of FGF20. For example, a method of treatment described herein may comprise administering to a subject a therapeutically effective amount of recombinant FGF20 (e.g., of human or mouse origin), a FGF20 analog (e.g., a mutated FGF20 polypeptide, or a FGF20 fusion protein, such as a chimeric protein containing FGF20 polypeptide and one or more polypeptide portions that enhance in vivo stability, in vivo half-life, and/or uptake/administration), a FGF20 mimetic (e.g., a non-peptide mimetic of FGF20), a FGF20 agonist (e.g., an agonistic FGF20 receptor antibody) and/or a nucleic acid molecule encoding FGF20.

Such therapeutic use of FGF20 may comprise the therapeutic use, as described, for example, in WO2005019427A2. WO2005019427A2 describes a method of treating a hyperphosphatemic condition by administering a therapeutically effective amount of an isolated FGF20 polypeptide (e.g., a FGF20 polypeptide with a mutation that confers increased stability to the FGF20 polypeptide). Also described in WO2005019427A2 is a method of treating a hyperphosphatemic condition by administering a therapeutically effective amount of a reagent that increases the level of FGF20 polypeptide. Also described in WO2005019427A2 is a method of treating a condition involving deposition of calcium and phosphate in the arteries or soft tissues of a subject by administering to the subject a therapeutically effective amount of FGF20 or a reagent that increases the level of FGF20 polypeptide.

Alternatively, therapeutic use of FGF20 may comprise the therapeutic use, as described, for example, in WO2020160468A1. WO2020160468A1 describes a method of treating a patient diagnosed as having a neurocognitive disorder (NCD) by providing to the patient one or more agents that collectively increase expression and/or activity of two or more proteins selected from a group that includes FGF20.

### SPARC

In some embodiments, a method of treatment described herein comprises therapeutic use of SPARC, such as administering to a subject a therapeutically effective amount of a protein or nucleic acid molecule that increases the expression and/or function of SPARC. For example, a method of treatment described herein may comprise administering to a subject a therapeutically effective amount of recombinant SPARC (e.g., of human or mouse origin), a SPARC analog (e.g., a mutated SPARC polypeptide, or a SPARC fusion protein, such as a chimeric protein containing SPARC polypeptide and one or more polypeptide portions that enhance in vivo stability, in vivo half-life, and/or uptake/administration), a SPARC mimetic (e.g., a non-peptide mimetic of SPARC), a SPARC agonist (e.g., an agonistic SPARC receptor antibody) and/or a nucleic acid molecule encoding SPARC.

Such therapeutic use of SPARC may comprise the therapeutic use, as described, for example, in WO2008128169A1. WO2008128169A1 describes compositions for treating a mammalian tumor comprising a therapeutically effective amount of SPARC polypeptide and therapeutically effective amount of a hydrophobic chemotherapeutic agent (e.g., a microtubule inhibitor, such as a taxane) in absence or presence of an angiogenesis inhibitor. The SPARC polypepide used in the compositions of WO2008128169A1 is either exogenous wild-type SPARC or exogenous mutant SPARC (having a mutation corresponding to a deletion of the third glutamine in the mature form of the human SPARC protein).

Therapeutic use of SPARC may also comprise the therapeutic use, as described, for example, in WO2013170365A1. WO2013170365A1 discloses a method for sensitization of cancer cells through the administration of SPARC polypeptide and GRP78. SPARC polypeptide used in the methods of WO2013170365A1 refers to full length 303 amino acid SPARC protein sequence and to any fragment or variant thereof, known in the art, that retains chemo-sensitzing activity, including a number of SPARC polypeptides described by Rahman et al. (PLOS ONE 10.1371/journal.pone.0026390 Published: 1 Nov. 2011), and SPARC fragments that were tested in WO/2008/000079.

Alternatively, therapeutic use of SPARC may comprise the therapeutic use, as described, for example, in Chlenski et al. (Mol Cancer 9:138 (2010)). Chlenski et al. describes SPARC peptides corresponding to the follistatin domain of the protein (FS-E), especially, peptide FSEC that corresponds to the C-terminal loops of FS-E, to have potent anti-angiogenic and anti-tumorigenic effects in neuroblastoma.

### CCL5

In some embodiments, a method of treatment described herein comprises therapeutic use of CCL5, such as administering to a subject a therapeutically effective amount of a protein or nucleic acid molecule that increases the expression and/or function of CCL5. For example, a method of treatment described herein may comprise administering to a subject a therapeutically effective amount of recombinant CCL5 (e.g., of human or mouse origin), a CCL5 analog (e.g., a mutated CCL5 polypeptide, or a CCL5 fusion protein, such as a chimeric protein containing CCL5 polypeptide and one or more polypeptide portions that enhance in vivo stability, in vivo half-life, and/or uptake/administration), a CCL5 mimetic (e.g., a non-peptide mimetic of CCL5), a CCL5 agonist (e.g., an agonistic CCL5 receptor antibody) and/or a nucleic acid molecule encoding CCL5.

Such therapeutic use of CCL5 may comprise the therapeutic use, as described, for example, in Bhat et al. (Front Immunol, 11: 1849 (2020)) and/or Xie et al. (PNAS 118 (9) e2017282118 (2021)). Bhat et al. describes strong CCL5 production following arenavirus lymphocytic choriomeningitis virus (LCMV) treatment. Xie et al. shows widespread expression of chemokine CCL5 following Ciliary neurotrophic factor (CNTF) gene therapy.

Alternatively, therapeutic use of CCL5 may comprise the therapeutic use, as described, for example, in WO2020068261A1. WO2020068261A1 describes immunomodulatory fusion proteins comprising a collagen-binding domain operably linked to an immunomodulatory domain, wherein the immunomodulatory domain comprises one or more chemokines, such as CCL5, and methods of using the same, for example, to treat cancer.

In other instances, therapeutic use of CCL5 may comprise the therapeutic use, as described, for example, in WO2020146857A1. WO2020146857A1 describes a ProteAse Released chemoKines protein (PARK) comprising a prochemokine moiety comprising a propeptide moiety fused to a chemokine moiety, wherein the chemokine moiety comprises a N-terminus and a C-terminus, and wherein the chemokine moiety comprises a chemokine amino acid sequence having at least 90% similarity to CCL5; and a targeting moiety linked to the prochemokine moiety, wherein the targeting moiety has a binding specificity to a tumor, fibrosis or Alzheimer's Disease associated antigen or receptor.

### APP

In some embodiments, a method of treatment described herein comprises therapeutic use of APP, such as administering to a subject a therapeutically effective amount of a protein or nucleic acid molecule that increases the expression and/or function of APP. For example, a method of treatment described herein may comprise administering to a subject a therapeutically effective amount of recombinant APP (e.g., of human or mouse origin), an APP analog (e.g., a mutated APP polypeptide, or an APP fusion protein, such as a chimeric protein containing APP polypeptide and one or more polypeptide portions that enhance in vivo stability, in vivo half-life, and/or uptake/administration), an APP mimetic (e.g., a non-peptide mimetic of APP), an APP agonist (e.g., an agonistic APP receptor antibody) and/or a nucleic acid molecule encoding APP.

Such therapeutic use of APP may comprise the therapeutic use, as described, for example, in WO2020201471A1. WO2020201471A1 describes a compound for use in the treatment or prevention of a liver disease, wherein the compound is a amyloid beta related protein, the amyloid beta related protein being selected from the group consisting of amyloid beta protein, a amyloid beta peptide derived therefrom, amyloid precursor protein (APP), a compound involved in the generation of an amyloid beta peptide from APP, or a compound inhibiting the degradation of the amyloid beta protein or of amyloid peptides derived therefrom. Amyloid precursor protein or “APP” refers to an integral membrane protein expressed in many tissues and concentrated in the synapses of neurons. APP is known as the precursor molecule whose proteolysis generates beta amyloid (Ab). In particular, the amyloid beta peptide derived from the amyloid beta protein is selected from the group consisting of amyloid beta 40, amyloid beta 42 and amyloid beta 38. Further, the compound involved in the generation of an amyloid beta peptide from APP can be an enzyme selected from alpha-, beta- (BACE1), gamma-secretases, preferably presenilin.

Alternatively, therapeutic use of APP may comprise the therapeutic use, as described, for example, in WO2020160468A1. WO2020160468A1 describes compositions and methods for treating a patient having or at risk of developing a neurocognitive disorder, such as Alzheimer's disease, Parkinson's disease, and/or a frontotemporal lobar dementia, by providing to the patient one or more agents that collectively increase expression and/or activity of two or more proteins selected from a group that comprises APP. APP and Amyloid-beta A4 protein include wild-type forms of the APP gene or protein, as well as variants (e.g., splice variants, truncations, concatemers, and fusion constructs, among others) of wild-type APP proteins and nucleic acids encoding the same.

### PF4

In some embodiments, a method of treatment described herein comprises therapeutic use of PF4, such as administering to a subject a therapeutically effective amount of a protein or nucleic acid molecule that increases the expression and/or function of PF4. For example, a method of treatment described herein may comprise administering to a subject a therapeutically effective amount of recombinant PF4 (e.g., of human or mouse origin), a PF4 analog (e.g., a mutated PF4 polypeptide, or a PF4 fusion protein, such as a chimeric protein containing PF4 polypeptide and one or more polypeptide portions that enhance in vivo stability, in vivo half-life, and/or uptake/administration), a PF4 mimetic (e.g., a non-peptide mimetic of PF4), a PF4 agonist (e.g., an agonistic PF4 receptor antibody) and/or a nucleic acid molecule encoding PF4.

Such therapeutic use of PF4 may comprise the therapeutic use, as described, for example, in WO2009117710A2. WO2009117710A2 describes a method for treating an MIF-mediated disorder by administering to a subject an active agent that inhibits (i) MIF binding to CXCR2 and CXCR4 and/or (ii) MIF-activation of CXCR2 and CXCR4; (iii) the ability of MIF to form a homomultimer; or a combination thereof, wherein the active agent can be recombinant PF4.

Alternatively, therapeutic use of PF4 may comprise the therapeutic use, as described, for example, in WO1994013321A1. WO1994013321A1 describes process for suppressing myeloid cells by administering a synergistic combination of chemokines which suppress myeloid cells, wherein the synergistic combination includes at least one chemokine selected from a group consisting of PF4. PF4 used in methods and compositions of WO1994013321A1 is natural human PF4.

### DNAJC19

In some embodiments, a method of treatment described herein comprises therapeutic use of DNAJC19, such as administering to a subject a therapeutically effective amount of a protein or nucleic acid molecule that increases the expression and/or function of DNAJC19. For example, a method of treatment described herein may comprise administering to a subject a therapeutically effective amount of recombinant DNAJC19 (e.g., of human or mouse origin), a DNAJC19 analog (e.g., a mutated DNAJC19 polypeptide, or a DNAJC19 fusion protein, such as a chimeric protein containing DNAJC19 polypeptide and one or more polypeptide portions that enhance in vivo stability, in vivo half-life, and/or uptake/administration), a DNAJC19 mimetic (e.g., a non-peptide mimetic of DNAJC19), a DNAJC19 agonist (e.g., an agonistic DNAJC19 receptor antibody) and/or a nucleic acid molecule encoding DNAJC19.

Such therapeutic use of DNAJC19 may comprise the therapeutic use, as described, for example, in WO2016170348A2. WO2016170348A2 describes small activating RNA for modulating the expression of a target gene for therapeutic purpose, wherein the target gene can be DNAJC19.

Alternatively, therapeutic use of DNAJC19 may comprise the therapeutic use, as described, for example, in WO2017191274A2. WO2017191274A2 describes RNA comprising coding sequence, useful for preparing composition used as medicament used in gene therapy in disease, disorder or condition, e.g. metabolic or endocrine disorders, cancer, infectious diseases or immunodeficiencies, wherein the encoded peptide or protein comprises a therapeutic protein or a fragment or variant thereof, selected from a group that includes, without limitation DNAJC19.

### Testican-2

In some embodiments, a method of treatment described herein comprises therapeutic use of Testican-2, such as administering to a subject a therapeutically effective amount of a protein or nucleic acid molecule that is or increases the expression and/or function of Testican-2. For example, a method of treatment described herein may comprise administering to a subject a therapeutically effective amount of recombinant Testican-2, a Testican-2 analog (e.g., a mutated Testican-2 polypeptide, or a Testican-2 fusion protein, such as a chimeric protein containing Testican-2 polypeptide and one or more polypeptide portions that enhance in vivo stability, in vivo half-life, and/or uptake/administration), a Testican-2 mimetic (e.g., a non-peptide mimetic of Testican-2), a Testican-2 agonist (e.g., an agonistic Testican-2 receptor antibody) and/or a nucleic acid molecule encoding Testican-2.

In certain embodiments, the methods and compositions disclosed herein are used to identify a human subject who is at risk of developing progressive renal decline (the subject may already have renal decline in which case the risk is assessed with respect to even further progression) where a therapy to improve kidney function (i.e., slow progression of kidney disease) is administered to the human subject who is identified as being at risk. Examples of therapy include, but are not limited to losing weight, an agent to control high blood pressure, and/or an agent to control high cholesterol levels. Such agents may be used to treat problems that may cause progressive kidney disease and the complications that can happen as a result of it, e.g., high blood pressure. The methods disclosed herein also include, in certain embodiments, administering an additional agent to the subject, for example an anti-fibrosis agent. Exemplary agents include, but are not limited to angiotensin-converting enzyme inhibitors (ACEI) and angiotensin II receptor type 1 blockers (ARB), renin inhibitors (aliskiren, enalkiren, zalkiren), mineralocorticoid receptor blockers (spironolacton, eplerenone), vasopeptidase inhibitors (e.g. AVE7688, omapatrilat). In certain embodiments, a statin, e.g., atorvastatin or simvastatin, is administered to lower cholesterol levels of the human subject.

Further, nucleic acid molecules (e.g., DNA and/or mRNA nucleic acid molecules) useful in the therapeutic methods described herein may be synthetic. The term “synthetic” means the nucleic acid molecule is isolated and not identical in sequence (the entire sequence) and/or chemical structure to a naturally-occurring nucleic acid molecule, such as an endogenou s precursor mRNA molecule. While in some embodiments, nucleic acids of the invention do not have an entire sequence that is identical to a sequence of a naturally-occurring nucleic acid, such molecules may encompass all or part of a naturally-occurring sequence. It is contemplated, however, that a synthetic nucleic acid administered to a cell may subsequently be modified or altered in the cell such that its structure or sequence is the same as non-synthetic or naturally occurring nucleic acid, such as a mature mRNA sequence. For example, a synthetic nucleic acid may have a sequence that differs from the sequence of a precursor mRNA, but that sequence may be altered once in a cell to be the same as an endogenous, processed mRNA. The term “isolated” means that the nucleic acid molecules of the disclosure are initially separated from different (in terms of sequence or structure) and unwanted nucleic acid molecules such that a population of isolated nucleic acids is at least about 90% homogenous, and may be at least about 95, 96, 97, 98, 99, or 100% homogenous with respect to other polynucleotide molecules. In many embodiments of the disclosure, a nucleic acid is isolated by virtue of it having been synthesized in vitro separate from endogenous nucleic acids in a cell. It will be understood, however, that isolated nucleic acids may be subsequently mixed or pooled together.

A nucleic acid may be made by any technique known to one of ordinary skill in the art, such as for example, chemical synthesis, enzymatic production or biological production.

Nucleic acid synthesis is performed according to standard methods. See, for example, Itakura and Riggs (1980). Additionally, U.S. Pat. Nos. 4,704,362, 5,221,619, and 5,583,013 each describe various methods of preparing synthetic nucleic acids. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR (see for example, U.S. Pat. Nos. 4,683,202 and 4,682,195, each incorporated herein by reference), or the synthesis of an oligonucleotide described in U.S. Pat. No. 5,645,897, incorporated herein by reference.

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Recombinant methods for producing nucleic acids in a cell are well known to those of skill in the art. These include the use of vectors, plasmids, cosmids, and other vehicles for delivery a nucleic acid to a cell, which may be the target cell or simply a host cell (to produce large quantities of the desired RNA molecule). Alternatively, such vehicles can be used in the context of a cell free system so long as the reagents for generating the RNA molecule are present. Such methods include those described in Sambrook, 2003, Sambrook, 2001 and Sambrook, 1989, which are hereby incorporated by reference.

In certain embodiments, the nucleic acid molecules of the present disclosure are not synthetic. In some embodiments, the nucleic acid molecule has a chemical structure of a naturally occurring nucleic acid and a sequence of a naturally occurring nucleic acid. In addition to the use of recombinant technology, such non-synthetic nucleic acids may be generated chemically, such as by employing technology used for creating oligonucleotides.

Administration or delivery of a therapeutic agent (e.g., a protective protein) according to the present disclosure may be via any route so long as the target tissue is available via that route. For example, administration may be by intradermal, subcutaneous, intramuscular, intraperitoneal or intravenous injection, or by direct injection into target tissue (e.g., cardiac tissue). Pharmaceutical compositions comprising polypeptides or polynucleotides or expression constructs comprising polypeptide or polynucleotide sequences may also be administered by catheter systems or systems that isolate coronary circulation for delivering therapeutic agents to the heart. Various catheter systems for delivering therapeutic agents to the heart and coronary vasculature are known in the art. Some non-limiting examples of catheter-based delivery methods or coronary isolation methods suitable for use in the present invention are disclosed in U.S. Pat. Nos. 6,416,510; 6,716,196; 6,953,466, WO 2005/082440, WO 2006/089340, U.S. Patent Publication No. 2007/0203445, U.S. Patent Publication No. 2006/0148742, and U.S. Patent Publication No. 2007/0060907, which are all hereby incorporated by reference in their entireties.

The a therapeutic agent (e.g., a protective protein) may also be administered parenterally or intraperitoneally. By way of illustration, solutions of the conjugates as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The a therapeutic agent (e.g., a protective protein) suitable for injectable use or catheter delivery include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agent(s), for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by their use in the compositions of agents delaying absorption, for example, aluminum monostearate and gelatin.

The disclosure is further illustrated by the following examples, which should not be construed as limiting.

### EXAMPLES

Described herein are studies that identify biomarkers useful for diagnosing, prognosing, and identifying subjects with, or suspected of having, or potentially developing progressive renal decline and/or ESKD. The following examples are included for purpose of illustration only and are not intended to be limiting.

Over the last several decades, considerable research efforts have been directed toward understanding the mechanisms of diabetic kidney disease (DKD) in humans with type 1 diabetes (T1D) as well as in type 2 diabetes (T2D). In that research, the major focus was on factors and markers that were associated with high risk of the development of various manifestations of DKD (Parving et al., Diabetic Nephropathy. In: Brenner BM, ed. Brenner and Rector's The Kidney. 7th ed. Philadelphia. (Elsevier, 2004); JAMA 290: 2159-2167 (2003); Lancet 352: 837-853 (1998); Nowak et al., Kidney International 93: 1198-1206 (2018); Niewczas et al., Nat Med 25: 805-813 (2019); Ahluwalia et al., Editorial: Novel Biomarkers for Type 2 Diabetes. Front Endocrinol (Lausanne) 10: 649 (2019)). Recent attention has focused on the search for factors and biomarkers associated with protection against DKD. It has been postulated that subjects who remained without late complications despite long duration of diabetes, so-called survivors with long diabetes duration, could be enriched for such protective factors/biomarkers. This approach has already provided findings that resulted not only in the development of a new hypothesis about DKD, but also in the identification of pyruvate kinase M2 (PKM2) as a new therapeutic target to prevent DKD (Qi et al., Nat Med 23: 753-762 (2017)).

### Materials and Methods

The subjects for the study described herein were selected from among participants of the Joslin Kidney Study (JKS). The Joslin Diabetes Center Committee on Human Studies approved the informed consent, recruitment and examination protocols for the JKS, a longitudinal observational study that investigates the determinants and natural history of kidney function decline in both types of diabetes.

### Joslin Kidney Study (JKS)

Briefly, the JKS comprises two components, type 1 diabetes (T1D) and type 2 diabetes (T2D). Subjects in the T1D component were recruited consecutively from among 3,500 adults 18-64 years old with T1D who attended the Joslin Clinic between 1991 and 2009. According to the median values of ACR obtained during the 2-year period preceding enrollment (baseline examination), subjects were classified into three sub-groups: those with Macro-Albuminuria (ACR≥300 μg/mg), Micro-Albuminuria (30≤ACR<300 μg/mg), and Normo-Albuminuria (ACR<30 μg/mg). The aim was to recruit into the JKS all of those with Macro- and Micro-Albuminuria and a similar number of subjects with Normo-Albuminuria. In total, 1884 subjects were enrolled: 526 with Macro-Albuminuria, 563 with Micro-albuminuria and 795 with Normo-Albuminuria.

Subjects in the T2D cohort were recruited consecutively from among 4500 adults 35-64 years old with T2D who attended the Joslin Clinic between 2003 and 2009. According to the median values of ACR obtained during the 2-year period preceding enrollment (baseline examination), subjects were classified into three sub-groups as described above for T1D. The aim was to recruit into the JKS all those with Macro- and Micro-Albuminuria and a similar number of subjects with Normo-Albuminuria. In total, 1,476 subjects were enrolled: 261 with Macro-Albuminuria, 482 with Micro-Albuminuria and 733 with Normo-Albuminuria.

All subjects enrolled into the JKS had biannual examinations either during routine clinic visits or were invited for a special visit or were examined at their homes. These examinations were conducted until they developed end-stage kidney disease (ESKD), died, were lost to follow-up or until the end of follow-up in 2015. Biospecimens obtained at examinations were stored in −85° C. Serum creatinine was used to determine kidney function at baseline and its changes during follow-up visits. Serum creatinine measurements were calibrated over time using protocols described by Skupien et al. (Kidney international 82: 589-597 (2012)). Estimates of glomerular filtration rate (GFR) were obtained using the Chronic Kidney Disease Epidemiology Collaboration formula, as described by Levey et al. (Ann Intern Med 150: 604-612 (2009)).

To classify patterns of trajectories of kidney function changes during follow-up, the first step was to determine whether they were linear or non-linear. Although most estimated glomerular filtration rate (eGFR) trajectories appeared linear on inspection, this impression was validated statistically by fitting both linear and spline models to each patient's kidney function trajectory. An approach described by Jones and Molitoris (Anal Biochem 141: 287-290 (1984)) and used by Shah and Levey (J Am Soc Nephrol 2: 1186-1191 (1992)) was applied to examine an individual's serial kidney function changes during follow-up. Participants in the study had 5 or more eGFR determinations over 7-15 years of follow-up. The method represents each participant's kidney function trajectory as a simple linear model and as a spline model with linear segments connected at an individually determined point. The linear and spline models were compared, and the linear model was rejected at a nominal significance of 0.05 and degrees of freedom determined by the number of spline segments (n−1). The majority had linear slopes. To determine the slope of eGFR decline, the linear component of each individual's trajectory was extracted to generate distribution of slopes of overall eGFR change during follow-up. Details of this approach are described below and also described in Skupien et al. (Kidney international 82: 589-597 (2012)).

All subjects included in the JKS were queried every two years against rosters of the United States Renal Data System (USRDS) and the National Death Index (NDI) to ascertain patients who developed ESKD or died. The last inquiries were conducted in 2015. The USRDS maintains a roster of US patients receiving renal replacement therapy, which includes dates of dialysis and transplantation.

### Exploratory, Replication and Validation Cohorts

The current study comprises three JKS cohorts; the exploratory cohort of 214 subjects with T1D and the replication cohort of 144 subjects with T2D, who previously participated in our study to determine cut-point values of serum TNF-R1 concentrations for the prediction of development of ESKD in T1D and T2D (Yamanouchi et al., Kidney International 92: 258-266 (2017)). In contrast to the previous study which included subjects with Chronic Kidney Disease (CKD) Stages 3 and 4, the present study included subjects in the JKS who had CKD Stage 3 at baseline examination. The validation cohort consists of 294 subjects with T1D who had CKD Stages 1 and 2 at baseline and was used to examine the importance of three exemplar protective proteins observed in late diabetic kidney disease (DKD) cohorts in subjects with an early stage of DKD. The primary goal was to search for protective proteins against progressive renal decline and progression to ESKD not only in T1D patients with impaired kidney function but also in any diabetic patients at any stages of DKD. Therefore, to demonstrate the robustness of the findings, three very different cohorts with different baseline characteristics were selected; the T1D exploratory (T1D patients with late stage of DKD), the T2D replication (T2D patients with late stage of DKD) and the T1D validation (T1D patients with early stage of DKD) cohorts.

Subjects with T1D and T2D had Macro- (ACR≥300 μg/mg) and Micro-albuminuria (ACR≥30 μg/mg). These subjects were followed for 7-15 years to determine the rate of eGFR decline (eGFR slopes) and to ascertain onset of ESKD. All clinical data and plasma specimens from these subjects were available for the current study. Detailed descriptions of these cohorts, measurements of clinical characteristics, determinations of eGFR slopes from serial measurements of serum creatinine, and ascertainment of onset of ESKD are described, for example, in Niewczas et al. (Nat Med 25: 805-813 (2019)) and Yamanouchi et al. (Kidney International 92: 258-266 (2017)). In all 3 cohorts, eGFR loss <3.0 ml/min/year were selected as the threshold to define those with slow (non-progressors) or fast (progressors) progressive renal decline. The rationale for such a threshold was well documented and used in previous publications (Perkins et al., J Am Soc Nephrol 18: 1353-1361 (2007); Krolewski et al., Diabetes Care 37: 226-234 (2014)) and corresponds to the 2.5^{th} percentile of the distribution of annual kidney function loss in a general population (Lindeman et al., J Am Geriatr Soc 33: 278-285 (1985)).

### Healthy Non-Diabetic Parents of T1D Subjects

During the Joslin Kidney Study, living parents of subjects with T1D were also examined. The group of non-diabetic parents of T1D subjects was derived from genetic study on determinants of DKD in T1D. Parents had baseline examinations performed according to the same protocols as all participants of the JKS. Biospecimens obtained at examinations were stored in −85° C. For the purpose of this study, 79 white non-diabetic parents aged 50-69 years at baseline examination were selected to be used as non-diabetic controls. Forty parents had children who remained without kidney complications despite long duration of diabetes and 39 parents had children who had advanced DKD (impaired kidney function or ESKD). The clinical phenotype of the T1D offspring of the non-diabetic parents is either normo-albuminuria (n=40), or ESKD or proteinuria (n=39). Plasma specimens obtained at baseline examination were subjected to the SOMAscan analysis.

### The SOMAscan Proteomic Analysis

The SOMAscan proteomic platform uses single-stranded DNA aptamers that measure 1129 protein concentrations in only 50 μl plasma, serum or equally small amounts of a variety of other biological matrices. A complete list of the proteins is provided in Table 1. The SOMAscan platform is facilitated by a new generation of the Slow Off-rate Modified Aptamer (SOMAMER) reagents that benefit from the aptamer technology developed over the past 20 years (Tuerk et al., Science 249: 505-510 (1990); Ellington et al., Nature 346: 818-822 (1990)). The SOMAmer reagents are selected against proteins in their native folded conformations and bind to folded proteins and thus three-dimensional shape epitopes rather than linear peptide sequences. The SOMAscan platform offers a remarkably dynamic range, and this large dynamic range results from the detection range of each SOMAMER reagent in combination with three serial dilutions of the sample of interest. The dilutions are separated into three pools: the 40% (the most concentrated sample to detect the least abundant proteins—fM to pM in 100% sample), 1% (mid-range) and 0.005% (the least concentrated sample designs to detect the most abundant proteins—˜μM in 100% sample). The assay readout is reported in relative fluorescent units (RFU) and is directly proportional to the target protein amount in the original sample. The details of the SOMAscan proteomics platform are described elsewhere (Gold et al., PLoS One 5: e15004 (2010); Hathout et al., Proc Natl Acad Sci USA 112: 7153-7158 (2015)).

Proteomic profiling was performed using the SOMAscan platform based at the SomaLogic laboratory (Boulder, CO). The Human Plasma SOMAscan 1.1 k kit with a set of calibration and normalization samples was used following the manufacturer's recommended protocol. Data standardization was performed according to the SOMAscan platform data quality-control protocols. To standardize SOMAscan assay results, raw SOMAscan assay data was first normalized to remove hybridization variation within a run (hybridization normalization) followed by median signal normalization across all samples to remove other assay biases within the run and finally calibrated to remove assay differences between runs. The acceptance criteria for hybridization and median signal normalization scale factors are expected to be in the range of 0.4-2.5. The median of the calibration scale factors is expected to be within ±0.2 from 1.0 and a minimum of 95% of individual SOMAmer reagents in the total array must be within ±0.4 from the median. SOMAscan data from all samples passed quality control criteria and were fit for analysis.

### Technical Validation of SOMAmer Specificity by LC-MS/MS

To systematically assess SOMAscan platform specificity, protocols using SOMAmer were developed for affinity pull-down of intact proteins followed by digestion to peptides and analysis by untargeted mass spectrometry. The FGF20 SOMAmer reagent was thawed, vortexed and spun down for 2 minutes (min), heated to 100° C. for 5 min in PCR machine, and then slowly cooled in 25° C. water bath. The FGF20 SOMAmer was diluted to 50 mM AB Buffer (40 mM HEPES, 100 mM NaCl, 5 mM KCl, 5 mM MgCl2, 0.05% Tween-20 at pH 7.5), and then cooled in a water bath to 25° C. for 20 min. Streptavidin Agarose beads were diluted from 50 mM to 7.5%, and then spun at 1000×g for 2 min. The 7.5% streptavidin agarose beads were washed with AB buffer, vortexed and centrifuged for 2 min at 1000×g. The liquid was vacuumed out and the washing was repeated once more for a total of two times. SOMAmers were added to the beads and incubated for 20 min with shaking at 25° C. The tubes were spun for 2 min at 1000×g and the liquid was removed by vacuum. The beads were washed twice with 0-W buffer, and then washed twice with AB Buffer. AB Buffer, plasma and serum samples, and recombinant proteins were added to the appropriate tubes, along with 30 μl of SOMAmers bound beads. These tubes were shaken for 1.5 hours at room temperature. After the incubation was completed, the tubes were spun down for 1 minute and the liquid was removed. The samples were washed once with 1-B blocker, shaken for 5 min at 800 rpm, and the liquid was removed. The samples were washed 6 times with AB buffer, and then frozen at −80° C. Four times the sample volume of acetone at −20° C. was added to each tube. The tubes were quickly vortexed and incubated—20° C. for 1 hour. The tubes were centrifuge for 10 min at 13,000×g, and the supernatant was vacuumed out.

An equal volume of 0.5 M ammonium carbonate pH 10.5 was added to each set of washed beads. Another equal volume of reduction/alkylation cocktail consisting of 2% (v/v) iodoethanol and 0.5% (v/v) triethylphosphine in 97.5% acetonitrile was then added to each sample. The solutions were capped and incubated for 1 hour at 37° C., after which they were speed-vacuumed to dryness. The resulting pellets were then redissolved in a trypsin solution (Pierce Trypsin Protease MS-Grade, in 100 mM Tris-HCl, pH 8.0). The digestion was carried out at 37° C. overnight, after which the solutions were desalted using μC18 ZipTips (Millipore). The digested samples were analyzed with a Thermo Q-Exactive mass spectrometer using a Thermo EASY-nLC HPLC system. The separation was carried out with a 75 μm×15 cm Thermo EASY-Spray C18 column. MS data were collected in data dependent acquisition mode with a full high resolution MS scan followed by MS/MS scans of the top 10 most intense precursor ions (within a mass range of 350-2000 m/z).

TABLE 1
A complete list of all proteins (n = 1,129) measured on the SOMAscan platform.

| | | | | Entrez Gene |
|---|---|---|---|---|
| SomaID | Target | Target Full Name | UniProt | Symbol |
| SL000002 | VEGF | Vascular endothelial growth factor A | P15692 | VEGFA |
| SL000003 | Angiogenin | Angiogenin | P03950 | ANG |
| SL000004 | bFGF | Fibroblast growth factor 2 | P09038 | FGF2 |
| SL000006 | PAI-1 | Plasminogen activator inhibitor 1 | P05121 | SERPINE1 |
| SL000007 | ER | Estrogen receptor | P03372 | ESR1 |
| SL000009 | ERBB2 | Receptor tyrosine-protein kinase erbB-2 | P04626 | ERBB2 |
| SL000017 | VWF | von Willebrand factor | P04275 | VWF |
| SL000019 | Apo A-I | Apolipoprotein A-I | P02647 | APOA1 |
| SL000020 | Apo B | Apolipoprotein B | P04114 | APOB |
| SL000021 | Insulin | Insulin | P01308 | INS |
| SL000022 | D-dimer | D-dimer | P02671 | FGA FGB FGG |
| | | | P02675 | |
| | | | P02679 | |
| SL000024 | TF | Tissue Factor | P13726 | F3 |
| SL000027 | COX-2 | Prostaglandin G/H synthase 2 | P35354 | PTGS2 |
| SL000038 | MCP-1 | C-C motif chemokine 2 | P13500 | CCL2 |
| SL000039 | IL-8 | Interleukin-8 | P10145 | CXCL8 |
| SL000045 | IGFBP-3 | Insulin-like growth factor-binding | P17936 | IGFBP3 |
| | | protein 3 | | |
| SL000047 | IGF-I | Insulin-like growth factor I | P05019 | IGF1 |
| SL000048 | Protein C | Vitamin K-dependent protein C | P04070 | PROC |
| SL000049 | Protein S | Vitamin K-dependent protein S | P07225 | PROS1 |
| SL000051 | CRP | C-reactive protein | P02741 | CRP |
| SL000053 | tPA | Tissue-type plasminogen activator | P00750 | PLAT |
| SL000055 | Cadherin E | Cadherin-1 | P12830 | CDH1 |
| SL000057 | Thymidine | Thymidine kinase, cytosolic | P04183 | TK1 |
| | kinase | | | |
| SL000062 | PSA | Prostate-specific antigen | P07288 | KLK3 |
| SL000064 | Kallikrein 7 | Kallikrein-7 | P49862 | KLK7 |
| SL000070 | Glypican 3 | Glypican-3 | P51654 | GPC3 |
| SL000076 | p27Kip1 | Cyclin-dependent kinase inhibitor 1B | P46527 | CDKN1B |
| SL000087 | IL-6 | Interleukin-6 | P05231 | IL6 |
| SL000088 | TGF-b2 | Transforming growth factor beta-2 | P61812 | TGFB2 |
| SL000089 | TGF-b3 | Transforming growth factor beta-3 | P10600 | TGFB3 |
| SL000104 | Bc1-2 | Apoptosis regulator Bcl-2 | P10415 | BCL2 |
| SL000124 | MMP-2 | 72 kDa type IV collagenase | P08253 | MMP2 |
| SL000125 | IL-1a | Interleukin-1 alpha | P01583 | IL1A |
| SL000130 | Cyclin B1 | G2/mitotic-specific cyclin-B1 | P14635 | CCNB1 |
| SL000131 | PCNA | Proliferating cell nuclear antigen | P12004 | PCNA |
| SL000133 | MIP-3a | C-C motif chemokine 20 | P78556 | CCL20 |
| SL000134 | Met | Hepatocyte growth factor receptor | P08581 | MET |
| SL000136 | AREG | Amphiregulin | P15514 | AREG |
| SL000138 | HB-EGF | Heparin-binding EGF-like growth factor | Q99075 | HBEGF |
| SL000139 | EPI | Epiregulin | O14944 | EREG |
| SL000142 | TS | Thymidylate synthase | P04818 | TYMS |
| SL000158 | PSMA | Glutamate carboxypeptidase 2 | Q04609 | FOLH1 |
| SL000164 | Myoglobin | Myoglobin | P02144 | MB |
| SL000247 | 6-Phospho- | 6-phosphogluconate dehydrogenase, | P52209 | PGD |
| | gluconate de | decarboxylate | | |
| SL000248 | a1-Antichymo- | Alpha-1-antichymotrypsin | P01011 | SERPINA3 |
| | trypsin | | | |
| SL000249 | a1- | Alpha-1-antitrypsin | P01009 | SERPINA1 |
| | Antitrypsin | | | |
| SL000250 | a2- | Alpha-2-antiplasmin | P08697 | SERPINF2 |
| | Antiplasmin | | | |
| SL000251 | a2-HS- | Alpha-2-HS-glycoprotein | P02765 | AHSG |
| | Glycoprotein | | | |
| SL000252 | a2-Macro- | Alpha-2-macroglobulin | P01023 | A2M |
| | globulin | | | |
| SL000254 | Albumin | Serum albumin | P02768 | ALB |
| SL000268 | Angiostatin | Angiostatin | P00747 | PLG |
| SL000271 | Angiotensinogen | Angiotensinogen | P01019 | AGT |
| SL000272 | Antithrombin | Antithrombin-III | P01008 | SERPINC1 |
| | III | | | |
| SL000276 | Apo E | Apolipoprotein E | P02649 | APOE |
| SL000277 | Apo E2 | Apolipoprotein E (isoform E2) | P02649 | APOE |
| SL000280 | GOT1 | Aspartate aminotransferase, cytoplasmic | P17174 | GOT1 |
| SL000283 | b2-Micro- | Beta-2-microglobulin | P61769 | B2M |
| | globulin | | | |
| SL000299 | b-ECGF | Fibroblast growth factor 1 | P05230 | FGF1 |
| SL000300 | b-Endorphin | Beta-endorphin | P01189 | POMC |
| SL000305 | b-NGF | beta-nerve growth factor | P01138 | NGF |
| SL000306 | BNP-32 | Brain natriuretic peptide 32 | P16860 | NPPB |
| SL000308 | C1-Esterase | Plasma protease C1 inhibitor | P05155 | SERPING1 |
| | Inhibitor | | | |
| SL000309 | C1q | Complement C1q subcomponent | P02745 | CIQA C1QB |
| | | | P02746 | |
| | | | P02747 | |
| SL000310 | C1r | Complement Clr subcomponent | P00736 | C1R |
| SL000311 | C1s | Complement Cls subcomponent | P09871 | C1S |
| SL000312 | C3 | Complement C3 | P01024 | C3 |
| SL000313 | C3a | C3a anaphylatoxin | P01024 | C3 |
| SL000314 | C3b | Complement C3b | P01024 | C3 |
| SL000316 | C4 | Complement C4 | P0C0L4 | C4A C4B |
| | | | P0C0L5 | |
| SL000318 | C4b | Complement C4b | P0C0L4 | C4A C4B |
| | | | P0C0L5 | |
| SL000319 | C5 | Complement C5 | P01031 | C5 |
| SL000320 | C5a | C5a anaphylatoxin | P01031 | C5 |
| SL000321 | C5b, 6 | Complement C5b-C6 complex | P01031 | C5 C6 |
| | Complex | | P13671 | |
| SL000322 | C6 | Complement component C6 | P13671 | C6 |
| SL000323 | C7 | Complement component C7 | P10643 | C7 |
| SL000324 | C8 | Complement component C8 | P07357 | C8A C8B C8G |
| | | | P07358 | |
| | | | P07360 | |
| SL000325 | C9 | Complement component C9 | P02748 | C9 |
| SL000337 | Calpain I | Calpain I | P07384 | CAPN1 CAPNS |
| | | | P04632 | |
| SL000338 | Calpastatin | Calpastatin | P20810 | CAST |
| SL000339 | carbonic | Carbonic anhydrase 2 | P00918 | CA2 |
| | anhydrase II | | | |
| SL000342 | Catalase | Catalase | P04040 | CAT |
| SL000343 | Cathepsin B | Cathepsin B | P07858 | CTSB |
| SL000344 | Cathepsin D | Cathepsin D | P07339 | CTSD |
| SL000345 | Cathepsin G | Cathepsin G | P08311 | CTSG |
| SL000346 | Cathepsin H | Cathepsin H | P09668 | CTSH |
| SL000347 | CBG | Corticosteroid-binding globulin | P08185 | SERPINA6 |
| SL000357 | Coagulation | Coagulation factor IX | P00740 | F9 |
| | Factor IX | | | |
| SL000358 | Coagulation | Coagulation Factor VII | P08709 | F7 |
| | Factor VI | | | |
| SL000360 | Coagulation | Coagulation Factor X | P00742 | F10 |
| | Factor X | | | |
| SL000377 | CK-BB | Creatine kinase B-type | P12277 | CKB |
| SL000382 | CK-MB | Creatine kinase M-type:Creatine kinase | P12277 | CKB CKM |
| | | B-type | P06732 | |
| SL000383 | CK-MM | Creatine kinase M-type | P06732 | CKM |
| SL000384 | CTLA-4 | Cytotoxic T-lymphocyte protein 4 | P16410 | CTLA4 |
| SL000396 | Cytochrome c | Cytochrome c | P99999 | CYCS |
| SL000398 | Cytochrome | Cytochrome P450 3A4 | P08684 | CYP3A4 |
| | P450 3A4 | | | |
| SL000401 | Elastase | Neutrophil elastase | P08246 | ELANE |
| SL000403 | Endostatin | Endostatin | P39060 | COL18A1 |
| SL000406 | Eotaxin | Eotaxin | P51671 | CCL11 |
| SL000408 | Epo | Erythropoietin | P01588 | EPO |
| SL000409 | ERK-1 | Mitogen-activated protein kinase 3 | P27361 | MAPK3 |
| SL000414 | Factor B | Complement factor B | P00751 | CFB |
| SL000415 | Factor H | Complement factor H | P08603 | CFH |
| SL000420 | Ferritin | Ferritin | P02794 | FTH1 FTL |
| | | | P02792 | |
| SL000424 | Fibrinogen | Fibrinogen | P02671 | FGA FGB FGG |
| | | | P02675 | |
| | | | P02679 | |
| SL000426 | Fibronectin | Fibronectin | P02751 | FN1 |
| SL000427 | Fractalkine/ | Fractalkine | P78423 | CX3CL1 |
| | CX3CL-1 | | | |
| SL000428 | FSH | Follicle stimulating hormone | P01215, | CGA FSHB |
| | | | P01225 | |
| SL000433 | Glucagon | Glucagon | P01275 | GCG |
| SL000437 | Haptoglobin, | Haptoglobin | P00738 | HP |
| | Mixed Ty | | | |
| SL000440 | Hemopexin | Hemopexin | P02790 | HPX |
| SL000441 | HGF | Hepatocyte growth factor | P14210 | HGF |
| SL000445 | HIV-2 Rev | Protein Rev_HV2BE | P18093 | Human-virus |
| SL000449 | HSP 40 | DnaJ homolog subfamily B member 1 | P25685 | DNAJB1 |
| SL000450 | HSP 60 | 60 kDa heat shock protein, | P10809 | HSPD1 |
| | | mitochondrial | | |
| SL000451 | HSP 70 | Heat shock 70 kDa protein 1A/1B | P08107 | HSPA1A |
| SL000456 | iC3b | Complement C3b, inactivated | P01024 | C3 |
| SL000458 | IFN-g R1 | Interferon gamma receptor 1 | P15260 | IFNGR1 |
| SL000460 | IgD | Immunoglobulin D | P01880 | IGHD IGK@ |
| SL000461 | IgE | Immunoglobulin E | P01854 | IGHE IGK@ I |
| SL000462 | IGFBP-1 | Insulin-like growth factor-binding | P08833 | IGFBP1 |
| | | protein 1 | | |
| SL000466 | IGFBP-2 | Insulin-like growth factor-binding | P18065 | IGFBP2 |
| | | protein 2 | | |
| SL000467 | IgG | Immunoglobulin G | P01857 | IGHG1 IGHG2 |
| SL000468 | IgM | Immunoglobulin M | P01871 | IGHM IGJ IG |
| SL000470 | IL-11 | Interleukin-11 | P20809 | IL11 |
| SL000474 | IL-16 | Interleukin-16 | Q14005 | IL16 |
| SL000478 | IL-2 | Interleukin-2 | P60568 | IL2 |
| SL000479 | IL-3 | Interleukin-3 | P08700 | IL3 |
| SL000480 | IL-4 | Interleukin-4 | P05112 | IL4 |
| SL000481 | IL-5 | Interleukin-5 | P05113 | IL5 |
| SL000483 | IL-7 | Interleukin-7 | P13232 | IL7 |
| SL000493 | LDH-H 1 | L-lactate dehydrogenase B chain | P07195 | LDHB |
| SL000496 | Lactoferrin | Lactotransferrin | P02788 | LTF |
| SL000497 | Laminin | Laminin | P25391 | LAMA1 LAMB1 |
| | | | P07942 | |
| | | | P11047 | |
| SL000498 | Leptin | Leptin | P41159 | LEP |
| SL000506 | Luteinizing | Luteinizing hormone | P01215 | CGA LHB |
| | hormone | | P01229 | |
| SL000507 | Lymphotoxin | Lymphotoxin alpha1:beta2 | P01374 | LTA LTB |
| | a1/b2 | | Q06643 | |
| SL000508 | Lymphotoxin | Lymphotoxin alpha2:beta1 | P01374 | LTA LTB |
| | a2/b1 | | Q06643 | |
| SL000509 | Lymphotoxin | Tumor necrosis factor receptor | P36941 | LTBR |
| | b R | superfamily me | | |
| SL000510 | Lysozyme | Lysozyme C | P61626 | LYZ |
| SL000515 | MCP-2 | C-C motif chemokine 8 | P80075 | CCL8 |
| SL000516 | MCP-3 | C-C motif chemokine 7 | P80098 | CCL7 |
| SL000517 | MCP-4 | C-C motif chemokine 13 | Q99616 | CCL13 |
| SL000519 | MIP-1a | C-C motif chemokine 3 | P10147 | CCL3 |
| SL000521 | MMP-1 | Interstitial collagenase | P03956 | MMP1 |
| SL000522 | MMP-12 | Macrophage metalloelastase | P39900 | MMP12 |
| SL000523 | MMP-13 | Collagenase 3 | P45452 | MMP13 |
| SL000524 | MMP-3 | Stromelysin-1 | P08254 | MMP3 |
| SL000525 | MMP-7 | Matrilysin | P09237 | MMP7 |
| SL000526 | MMP-8 | Neutrophil collagenase | P22894 | MMP8 |
| SL000527 | MMP-9 | Matrix metalloproteinase-9 | P14780 | MMP9 |
| SL000528 | NADPH- | NADPH--cytochrome P450 reductase | P16435 | POR |
| | P450 | | | |
| | Oxidoreduc | | | |
| SL000530 | OSM | Oncostatin-M | P13725 | OSM |
| SL000532 | ON | SPARC | P09486 | SPARC |
| SL000535 | PDGF-AA | Platelet-derived growth factor subunit A | P04085 | PDGFA |
| SL000537 | PDGF-BB | Platelet-derived growth factor subunit B | P01127 | PDGFB |
| SL000539 | PHI | Glucose-6-phosphate isomerase | P06744 | GPI |
| SL000540 | Plasmin | Plasmin | P00747 | PLG |
| SL000541 | Plasminogen | Plasminogen | P00747 | PLG |
| SL000542 | gpIIbIIIa | Integrin alpha-IIb:beta-3 complex | P08514 | ITGA2B ITGB |
| | | | P05106 | |
| SL000545 | Prekallikrein | Plasma kallikrein | P03952 | KLKB1 |
| SL000546 | PRL | Prolactin | P01236 | PRL |
| SL000550 | PCI | Plasma serine protease inhibitor | P05154 | SERPINA5 |
| SL000551 | PKC-A | Protein kinase C alpha type | P17252 | PRKCA |
| SL000553 | PKC-B-II | Protein kinase C beta type | P05771 | PRKCB |
| SL000554 | PKC-D | Protein kinase C delta type | Q05655 | PRKCD |
| SL000556 | PKC-G | Protein kinase C gamma type | P05129 | PRKCG |
| SL000557 | PKC-Z | Protein kinase C zeta type | Q05513 | PRKCZ |
| SL000558 | Prothrombin | Prothrombin | P00734 | F2 |
| SL000560 | P-Selectin | P-Selectin | P16109 | SELP |
| SL000563 | RANTES | C-C motif chemokine 5 | P13501 | CCL5 |
| SL000565 | Renin | Renin | P00797 | REN |
| SL000566 | RBP | Retinol-binding protein 4 | P02753 | RBP4 |
| SL000570 | Secretin | Secretin | P09683 | SCT |
| SL000572 | SAA | Serum amyloid A-1 protein | P0DJI8 | SAA1 |
| SL000573 | SAP | Serum amyloid P-component | P02743 | APCS |
| SL000581 | SOD | Superoxide dismutase [Cu—Zn] | P00441 | SOD1 |
| SL000582 | Survivin | Baculoviral IAP repeat-containing | O15392 | BIRC5 |
| | | protein 5 | | |
| SL000584 | TGF-b1 | Transforming growth factor beta-1 | P01137 | TGFB1 |
| SL000586 | Thrombin | Thrombin | P00734 | F2 |
| SL000587 | Thyroglobulin | Thyroglobulin | P01266 | TG |
| SL000588 | TMA | Thyroid peroxidase | P07202 | TPO |
| SL000589 | TSH | Thyroid Stimulating Hormone | P01215 | CGA TSHB |
| | | | P01222 | |
| SL000590 | Thyroxine- | Thyroxine-Binding Globulin | P05543 | SERPINA7 |
| | Binding | | | |
| | Globulin | | | |
| SL000591 | TIMP-1 | Metalloproteinase inhibitor 1 | P01033 | TIMP1 |
| SL000592 | TIMP-2 | Metalloproteinase inhibitor 2 | P16035 | TIMP2 |
| SL000597 | TNF-b | Lymphotoxin-alpha | P01374 | LTA |
| SL000601 | Transferrin | Serotransferrin | P02787 | TF |
| SL000603 | Trypsin | Trypsin-1 | P07477 | PRSS1 |
| SL000605 | Ubiquitin + 1 | Ubiquitin + 1, truncated mutation for UbB | P62979 | RPS27A |
| SL000613 | uPA | Urokinase-type plasminogen activator | P00749 | PLAU |
| SL000615 | Vasoactive | Vasoactive Intestinal Peptide | P01282 | VIP |
| | Intestinal | | | |
| SL000617 | ALT | Alanine aminotransferase 1 | P24298 | GPT |
| SL000622 | Coagulation | Coagulation Factor V | P12259 | F5 |
| | Factor V | | | |
| SL000633 | Fas ligand, | Tumor necrosis factor ligand | P48023 | FASLG |
| | soluble | superfamily member 6, soluble form | | |
| SL000638 | Cadherin-2 | Cadherin-2 | P19022 | CDH2 |
| SL000640 | Nidogen | Nidogen-1 | P14543 | NID1 |
| SL000645 | MMP-10 | Stromelysin-2 | P09238 | MMP10 |
| SL000655 | Keratin 18 | Keratin, type I cytoskeletal 18 | P05783 | KRT18 |
| SL000658 | GAS1 | Growth arrest-specific protein 1 | P54826 | GAS1 |
| SL000668 | CD36 | Platelet glycoprotein 4 | P16671 | CD36 |
| | ANTIGEN | | | |
| SL000670 | GSTA3 | Glutathione S-transferase A3 | Q16772 | GSTA3 |
| SL000674 | FST | Follistatin | P19883 | FST |
| SL000678 | Granulysin | Granulysin | P22749 | GNLY |
| SL000695 | Lipocalin 2 | Neutrophil gelatinase-associated | P80188 | LCN2 |
| | | lipocalin | | |
| SL000836 | Hemoglobin | Hemoglobin | P69905, | HBA1 HBB |
| | | | P68871 | |
| SL001691 | FGF7 | Fibroblast growth factor 7 | P21781 | FGF7 |
| SL001713 | IL-17 | Interleukin-17A | Q16552 | IL17A |
| SL001716 | IL-12 | Interleukin-12 | P29459, | IL12A IL12B |
| | | | P29460 | |
| SL001717 | IL-10 | Interleukin-10 | P22301 | IL10 |
| SL001718 | IL-13 | Interleukin-13 | P35225 | IL13 |
| SL001720 | VCAM-1 | Vascular cell adhesion protein 1 | P19320 | VCAM1 |
| SL001721 | PECAM-1 | Platelet endothelial cell adhesion | P16284 | PECAM1 |
| | | molecule | | |
| SL001726 | GM-CSF | Granulocyte-macrophage colony- | P04141 | CSF2 |
| | | stimulating factor | | |
| SL001729 | G-CSF | Granulocyte colony-stimulating factor | P09919 | CSF3 |
| SL001737 | STRATIFIN | 14-3-3 protein sigma | P31947 | SFN |
| SL001753 | Sialoadhesin | Sialoadhesin | Q9BZZ2 | SIGLEC1 |
| SL001761 | Troponin I | Troponin I, cardiac muscle | P19429 | TNNI3 |
| SL001766 | HCG | Human Chorionic Gonadotropin | P01215, | CGA CGB |
| | | | P01233 | |
| SL001774 | FABP | Fatty acid-binding protein, heart | P05413 | FABP3 |
| SL001777 | Cystatin C | Cystatin-C | P01034 | CST3 |
| SL001795 | IL-1b | Interleukin-1 beta | P01584 | IL1B |
| SL001796 | Myeloper- | Myeloperoxidase | P05164 | MPO |
| | oxidase | | | |
| SL001797 | Kallikrein 6 | Kallikrein-6 | Q92876 | KLK6 |
| SL001800 | TNF sR-II | Tumor necrosis factor receptor | P20333 | TNFRSF1B |
| | | superfamily member 1B | | |
| SL001802 | IFN-g | Interferon gamma | P01579 | IFNG |
| SL001815 | Mn SOD | Superoxide dismutase [Mn], | P04179 | SOD2 |
| | | mitochondrial | | |
| SL001888 | SLPI | Antileukoproteinase | P03973 | SLPI |
| SL001890 | GA733-1 | Tumor-associated calcium signal | P09758 | TACSTD2 |
| | protein | transducer 2 | | |
| SL001896 | Clusterin | Clusterin | P10909 | CLU |
| SL001897 | SPINT2 | Kunitz-type protease inhibitor 2 | O43291 | SPINT2 |
| SL001902 | BCAM | Basal Cell Adhesion Molecule | P50895 | BCAM |
| SL001905 | Mesothelin | Mesothelin | Q13421 | MSLN |
| SL001938 | Activin A | Inhibin beta A chain | P08476 | INHBA |
| SL001943 | IL-6 sRa | Interleukin-6 receptor subunit alpha | P08887 | IL6R |
| SL001945 | sE-Selectin | E-Selectin | P16581 | SELE |
| SL001947 | MIA | Melanoma-derived growth regulatory | Q16674 | MIA |
| | | protein | | |
| SL001973 | Mammaglobin 2 | Mammaglobin-B | O75556 | SCGB2A1 |
| SL001992 | TNF sR-I | Tumor necrosis factor receptor | P19438 | TNFRSF1A |
| | | superfamily member 1A | | |
| SL001995 | Angiopoietin-1 | Angiopoietin-1 | Q15389 | ANGPT1 |
| SL001996 | Angiopoietin-2 | Angiopoietin-2 | O15123 | ANGPT2 |
| SL001997 | IL-1 sRI | Interleukin-1 receptor type 1 | P14778 | IL1R1 |
| SL001998 | TFPI | Tissue factor pathway inhibitor | P10646 | TFPI |
| SL001999 | MDM2 | E3 ubiquitin-protein ligase Mdm2 | Q00987 | MDM2 |
| SL002036 | FGFR4 | Fibroblast growth factor receptor 4 | P22455 | FGFR4 |
| SL002075 | IFN-aA | Interferon alpha-2 | P01563 | IFNA2 |
| SL002077 | Alkaline | Alkaline phosphatase, tissue-nonspecific | P05186 | ALPL |
| | phosphatase, | isozyme | | |
| | bone | | | |
| SL002078 | TGF-b R II | TGF-beta receptor type-2 | P37173 | TGFBR2 |
| SL002081 | Cadherin-5 | Cadherin-5 | P33151 | CDH5 |
| SL002086 | Ficolin-3 | Ficolin-3 | O75636 | FCN3 |
| SL002093 | Histone | Histone H2A.z | P0C0S5 | H2AFZ |
| | H2A.z | | | |
| SL002505 | ANP | Atrial natriuretic factor | P01160 | NPPA |
| SL002506 | suPAR | Urokinase plasminogen activator surface | Q03405 | PLAUR |
| | | receptor | | |
| SL002508 | IL-18 BPa | Interleukin-18-binding protein | O95998 | IL18BP |
| SL002517 | TNF-a | Tumor necrosis factor | P01375 | TNF |
| SL002519 | ERBB3 | Receptor tyrosine-protein kinase erbB-3 | P21860 | ERBB3 |
| SL002522 | Rb | Retinoblastoma-associated protein | P06400 | RB1 |
| SL002524 | sCD4 | T-cell surface glycoprotein CD4 | P01730 | CD4 |
| SL002525 | C2 | Complement C2 | P06681 | C2 |
| SL002528 | NPS-PLA2 | Phospholipase A2, membrane associated | P14555 | PLA2G2A |
| SL002539 | OPG | Tumor necrosis factor receptor | O00300 | TNFRSF11B |
| | | superfamily me | | |
| SL002541 | sRANKL | Tumor necrosis factor ligand | O14788 | TNFSF11 |
| | | superfamily member 11 | | |
| SL002542 | K-ras | GTPase KRas | P01116 | KRAS |
| SL002561 | PTHrP | Parathyroid hormone-related protein | P12272 | PTHLH |
| SL002621 | Midkine | Midkine | P21741 | MDK |
| SL002640 | PlGF | Placenta growth factor | P49763 | PGF |
| SL002644 | ERBB1 | Epidermal growth factor receptor | P00533 | EGFR |
| SL002646 | MMP-14 | Matrix metalloproteinase-14 | P50281 | MMP14 |
| SL002650 | M2-PK | Pyruvate kinase PKM | P14618 | PKM2 |
| SL002654 | Epithelial | Ephrin type-A receptor 2 | P29317 | EPHA2 |
| | cell kinas | | | |
| SL002655 | CTGF | Connective tissue growth factor | P29279 | CTGF |
| SL002662 | Coagulation | Coagulation Factor XI | P03951 | F11 |
| | Factor XI | | | |
| SL002684 | CSF-1 | Macrophage colony-stimulating factor 1 | P09603 | CSF1 |
| SL002695 | Glutamate | Cytosolic non-specific dipeptidase | Q96KP4 | CNDP2 |
| | carboxy- | | | |
| | peptidase | | | |
| SL002702 | PIM1 | Serine/threonine-protein kinase pim-1 | P11309 | PIM1 |
| SL002704 | PTN | Pleiotrophin | P21246 | PTN |
| SL002705 | Thrombo- | Thrombospondin-1 | P07996 | THBS1 |
| | spondin-1 | | | |
| SL002706 | CD23 | Low affinity immunoglobulin epsilon Fc | P06734 | FCER2 |
| | | receptor | | |
| SL002755 | PAPP-A | Pappalysin-1 | Q13219 | PAPPA |
| SL002756 | hnRNP K | Heterogeneous nuclear | P61978 | HNRNPK |
| | | ribonucleoprotein K | | |
| SL002763 | Kallikrein 11 | Kallikrein-11 | Q9UBX7 | KLK11 |
| SL002783 | Cardiotrophin-1 | Cardiotrophin-1 | Q16619 | CTF1 |
| SL002792 | BARK1 | beta-adrenergic receptor kinase 1 | P25098 | ADRBK1 |
| SL002803 | PGP9.5 | Ubiquitin carboxyl-terminal hydrolase | P09936 | UCHL1 |
| | | isozyme | | |
| SL002823 | sL-Selectin | L-Selectin | P14151 | SELL |
| SL002922 | sICAM-1 | Intercellular adhesion molecule 1 | P05362 | ICAM1 |
| SL003041 | PF-4 | Platelet factor 4 | P02776 | PF4 |
| SL003043 | TIMP-3 | Metalloproteinase inhibitor 3 | P35625 | TIMP3 |
| SL003060 | bFGF-R | Fibroblast growth factor receptor 1 | P11362 | FGFR1 |
| SL003080 | MIF | Macrophage migration inhibitory factor | P14174 | MIF |
| SL003104 | Eotaxin-2 | C-C motif chemokine 24 | O00175 | CCL24 |
| SL003166 | ALCAM | CD166 antigen | Q13740 | ALCAM |
| SL003167 | BLC | C-X-C motif chemokine 13 | O43927 | CXCL13 |
| SL003168 | CTACK | C-C motif chemokine 27 | Q9Y4X3 | CCL27 |
| SL003169 | ENA-78 | C-X-C motif chemokine 5 | P42830 | CXCL5 |
| SL003171 | FGF-4 | Fibroblast growth factor 4 | P08620 | FGF4 |
| SL003172 | GCP-2 | C-X-C motif chemokine 6 | P80162 | CXCL6 |
| SL003173 | Gro-a | Growth-regulated alpha protein | P09341 | CXCL1 |
| SL003176 | I-309 | C-C motif chemokine 1 | P22362 | CCL1 |
| SL003177 | sICAM-2 | Intercellular adhesion molecule 2 | P13598 | ICAM2 |
| SL003178 | sICAM-3 | Intercellular adhesion molecule 3 | P32942 | ICAM3 |
| SL003179 | Integrin | Integrin alpha-I:beta-1 complex | P56199 | ITGA1 ITGB1 |
| | a1b1 | | P05556 | |
| SL003182 | Integrin | Integrin alpha-V:beta-5 complex | P06756 | ITGAV ITGB5 |
| | aVb5 | | P18084 | |
| SL003183 | IP-10 | C-X-C motif chemokine 10 | P02778 | CXCL10 |
| SL003184 | sLeptin R | Leptin receptor | P48357 | LEPR |
| SL003186 | Lymphotactin | Lymphotactin | P47992 | XCL1 |
| SL003187 | MDC | C-C motif chemokine 22 | O00626 | CCL22 |
| SL003189 | MIP-3b | C-C motif chemokine 19 | Q99731 | CCL19 |
| SL003190 | MIP-5 | C-C motif chemokine 15 | Q16663 | CCL15 |
| SL003191 | NAP-2 | Neutrophil-activating peptide 2 | P02775 | PPBP |
| SL003192 | Properdin | Properdin | P27918 | CFP |
| SL003193 | 6Ckine | C-C motif chemokine 21 | O00585 | CCL21 |
| SL003196 | TARC | C-C motif chemokine 17 | Q92583 | CCL17 |
| SL003197 | TECK | C-C motif chemokine 25 | O15444 | CCL25 |
| SL003198 | Tenascin | Tenascin | P24821 | TNC |
| SL003199 | sTie-1 | Tyrosine-protein kinase receptor Tie-1, | P35590 | TIE1 |
| | | soluble | | |
| SL003200 | sTie-2 | Angiopoietin-1 receptor, soluble | Q02763 | TEK |
| SL003201 | VEGF sR2 | Vascular endothelial growth factor | P35968 | KDR |
| | | receptor 2 | | |
| SL003220 | C3adesArg | C3a anaphylatoxin des Arginine | P01024 | C3 |
| SL003280 | HMG-1 | High mobility group protein B1 | P09429 | HMGB1 |
| SL003300 | HCC-4 | C-C motif chemokine 16 | O15467 | CCL16 |
| SL003301 | Ck-b-8-1 | Ck-beta-8-1 | P55773 | CCL23 |
| SL003302 | MPIF-1 | C-C motif chemokine 23 | P55773 | CCL23 |
| SL003303 | CCL28 | C-C motif chemokine 28 | Q9NRJ3 | CCL28 |
| SL003304 | IGF-I sR | Insulin-like growth factor 1 receptor | P08069 | IGF1R |
| SL003305 | IL-2 sRa | Interleukin-2 receptor subunit alpha | P01589 | IL2RA |
| SL003307 | IL-2 sRg | Cytokine receptor common subunit | P31785 | IL2RG |
| | | gamma | | |
| SL003308 | IL-4 sR | Interleukin-4 receptor subunit alpha | P24394 | IL4R |
| SL003309 | LBP | Lipopolysaccharide-binding protein | P18428 | LBP |
| SL003310 | VEGF121 | Vascular endothelial growth factor A, | P15692 | VEGFA |
| | | isoform | | |
| SL003322 | VEGF sR3 | Vascular endothelial growth factor | P35916 | FLT4 |
| | | receptor 3 | | |
| SL003323 | PARC | C-C motif chemokine 18 | P55774 | CCL18 |
| SL003324 | Coagulation | Coagulation factor Xa | P00742 | F10 |
| | Factor Xa | | | |
| SL003326 | I-TAC | C-X-C motif chemokine 11 | O14625 | CXCL11 |
| SL003327 | Factor D | Complement factor D | P00746 | CFD |
| SL003328 | Factor I | Complement factor I | P05156 | CFI |
| SL003329 | HCC-1 | C-C motif chemokine 14 | Q16627 | CCL14 |
| SL003331 | MMP-16 | Matrix metalloproteinase-16 | P51512 | MMP16 |
| SL003332 | MMP-17 | Matrix metalloproteinase-17 | Q9ULZ9 | MMP17 |
| SL003334 | EMAP-2 | Endothelial monocyte-activating | Q12904 | AIMP1 |
| | | polypeptide 2 | | |
| SL003341 | Fibrinogen | Fibrinogen gamma chain | P02679 | FGG |
| | g-chain di | | | |
| SL003362 | C3d | Complement C3d fragment | P01024 | C3 |
| SL003440 | PAFAH | Platelet-activating factor acetylhydrolase | Q13093 | PLA2G7 |
| SL003461 | ACTH | Corticotropin | P01189 | POMC |
| SL003520 | calreticulin | Calreticulin | P27797 | CALR |
| SL003522 | ERP29 | Endoplasmic reticulum resident protein | P30040 | ERP29 |
| | | 29 | | |
| SL003524 | Protein | Protein disulfide-isomerase A3 | P30101 | PDIA3 |
| | disulfide iso | | | |
| SL003542 | NG36 | Histone-lysine N-methyltransferase | Q96KQ7 | EHMT2 |
| | | EHMT2 | | |
| SL003643 | Glutathione | Glutathione S-transferase P | P09211 | GSTP1 |
| | S-transfe | | | |
| SL003647 | annexin VI | Annexin A6 | P08133 | ANXA6 |
| SL003648 | Rab GDP | Rab GDP dissociation inhibitor beta | P50395 | GDI2 |
| | dissociation | | | |
| SL003653 | phospho- | Phosphoglycerate kinase 1 | P00558 | PGK1 |
| | glycerate kina | | | |
| SL003655 | Transketolase | Transketolase | P29401 | TKT |
| SL003657 | Calcineurin | Calcineurin | Q08209 | PPP3CA PPP3 |
| | | | P63098 | |
| SL003658 | Aflatoxin B1 | Aflatoxin B1 aldehyde reductase | O43488 | AKR7A2 |
| | aldehyde | member 2 | | |
| SL003674 | BCL2-like | Bcl-2-like protein 1 | Q07817 | BCL2L1 |
| | 1 protein | | | |
| SL003679 | IGF-II | Cation-independent mannose-6- | P11717 | IGF2R |
| | receptor | phosphate recept | | |
| SL003680 | sRAGE | Advanced glycosylation end product- | Q15109 | AGER |
| | | specific r | | |
| SL003685 | PBEF | Nicotinamide phosphoribosyltransferase | P43490 | NAMPT |
| SL003687 | Nucleoside | Nucleoside diphosphate kinase A | P15531 | NME1 |
| | diphosphate | | | |
| | kinase A | | | |
| SL003690 | RANK | Tumor necrosis factor receptor | Q9Y6Q6 | TNFRSF11A |
| | | superfamily member 11A | | |
| SL003703 | BFL1 | Bcl-2-related protein A1 | Q16548 | BCL2A1 |
| SL003710 | Caspase-2 | Caspase-2 | P42575 | CASP2 |
| SL003711 | Caspase-3 | Caspase-3 | P42574 | CASP3 |
| SL003717 | Caspase-10 | Caspase-10 | Q92851 | CASP10 |
| SL003726 | Chk2 | Serine/threonine-protein kinase Chk2 | O96017 | CHEK2 |
| SL003728 | cIAP-2 | Baculoviral IAP repeat-containing | Q13489 | BIRC3 |
| | | protein 3 | | |
| SL003733 | SMAC | Diablo homolog, mitochondrial | Q9NR28 | DIABLO |
| SL003735 | 4-1BB | Tumor necrosis factor ligand | P41273 | TNFSF9 |
| | ligand | superfamily member 9 | | |
| SL003738 | B7 | T-lymphocyte activation antigen CD80 | P33681 | CD80 |
| SL003739 | DcR3 | Tumor necrosis factor receptor | O95407 | TNFRSF6B |
| | | superfamily me | | |
| SL003744 | Galectin-3 | Galectin-3 | P17931 | LGALS3 |
| SL003753 | DLC8 | Dynein light chain 1, cytoplasmic | P63167 | DYNLL1 |
| SL003761 | pTEN | Phosphatidylinositol 3,4,5-trisphosphate | P60484 | PTEN |
| | | 3-ph | | |
| SL003764 | NCAM-120 | Neural cell adhesion molecule 1, 120 | P13591 | NCAM1 |
| | | kDa isoform | | |
| SL003770 | SARP-2 | Secreted frizzled-related protein 1 | Q8N474 | SFRP1 |
| SL003785 | GAPDH, | Glyceraldehyde-3-phosphate | P04406 | GAPDH |
| | liver | dehydrogenase | | |
| SL003793 | MEK1 | Dual specificity mitogen-activated | Q02750 | MAP2K1 |
| | | protein kinase | | |
| SL003800 | Kallikrein 4 | Kallikrein-4 | Q9Y5K2 | KLK4 |
| SL003803 | ERBB4 | Receptor tyrosine-protein kinase erbB-4 | Q15303 | ERBB4 |
| SL003849 | FGF9 | Fibroblast growth factor 9 | P31371 | FGF9 |
| SL003862 | CD40 | CD40 ligand | P29965 | CD40LG |
| | ligand, | | | |
| | soluble | | | |
| SL003863 | kallikrein 5 | Kallikrein-5 | Q9Y337 | KLK5 |
| SL003872 | gp130, | Interleukin-6 receptor subunit beta | P40189 | IL6ST |
| | soluble | | | |
| SL003915 | kallikrein 8 | Kallikrein-8 | O60259 | KLK8 |
| SL003916 | kallikrein 12 | Kallikrein-12 | Q9UKR0 | KLK12 |
| SL003918 | kallikrein 13 | Kallikrein-13 | Q9UKR3 | KLK13 |
| SL003919 | kallikrein 14 | Kallikrein-14 | Q9P0G3 | KLK14 |
| SL003930 | HPG- | 15-hydroxyprostaglandin dehydrogenase | P15428 | HPGD |
| | | [NAD(+)] | | |
| SL003951 | BDNF | Brain-derived neurotrophic factor | P23560 | BDNF |
| SL003970 | PTH | Parathyroid hormone | P01270 | PTH |
| SL003974 | Activated | Activated Protein C | P04070 | PROC |
| | Protein C | | | |
| SL003990 | FGFR-2 | Fibroblast growth factor receptor 2 | P21802 | FGFR2 |
| SL003993 | BMP-6 | Bone morphogenetic protein 6 | P22004 | BMP6 |
| SL003994 | BMP-1 | Bone morphogenetic protein 1 | P13497 | BMP1 |
| SL004008 | Proteinase-3 | Myeloblastin | P24158 | PRTN3 |
| SL004009 | RAC1 | Ras-related C3 botulinum toxin substrate | P63000 | RAC1 |
| | | 1 | | |
| SL004010 | SCF sR | Mast/stem cell growth factor receptor | P10721 | KIT |
| | | Kit | | |
| SL004015 | TAFI | Carboxypeptidase B2 | Q96IY4 | CPB2 |
| SL004016 | CXCL16, | C-X-C motif chemokine 16 | Q9H2A7 | CXCL16 |
| | soluble | | | |
| SL004060 | Endothelin- | Endothelin-converting enzyme 1 | P42892 | ECE1 |
| | converting | | | |
| SL004063 | FGFR-3 | Fibroblast growth factor receptor 3 | P22607 | FGFR3 |
| SL004064 | GIB | Phospholipase A2 | P04054 | PLA2G1B |
| SL004066 | GIIE | Group IIE secretory phospholipase A2 | Q9NZK7 | PLA2G2E |
| SL004067 | GX | Group 10 secretory phospholipase A2 | O15496 | PLA2G10 |
| SL004068 | Granzyme B | Granzyme B | P10144 | GZMB |
| SL004070 | Ubiquitin | Ubiquitin | P62979 | RPS27A |
| SL004078 | BMP-7 | Bone morphogenetic protein 7 | P18075 | BMP7 |
| SL004080 | BMPR1A | Bone morphogenetic protein receptor | P36894 | BMPR1A |
| | | type-1A | | |
| SL004081 | Bone | Decorin | P07585 | DCN |
| | proteoglycan | | | |
| | II | | | |
| SL004118 | TrATPase | Tartrate-resistant acid phosphatase type | P13686 | ACP5 |
| | | 5 | | |
| SL004119 | discoidin | Epithelial discoidin domain-containing | Q08345 | DDR1 |
| | domain | receptor 1 | | |
| | receptor 1 | | | |
| SL004120 | Discoidin | Discoidin domain-containing receptor 2 | Q16832 | DDR2 |
| | domain | | | |
| | receptor 2 | | | |
| SL004125 | IR | Insulin receptor | P06213 | INSR |
| SL004126 | 4-1BB | Tumor necrosis factor receptor | Q07011 | TNFRSF9 |
| | | superfamily member 9 | | |
| SL004128 | Activin RIB | Activin receptor type-1B | P36896 | ACVR1B |
| SL004131 | B7-2 | T-lymphocyte activation antigen CD86 | P42081 | CD86 |
| SL004133 | BMP RII | Bone morphogenetic protein receptor | Q13873 | BMPR2 |
| | | type-2 | | |
| SL004134 | CD27 | CD27 antigen | P26842 | CD27 |
| SL004136 | Dtk | Tyrosine-protein kinase receptor TYRO3 | Q06418 | TYRO3 |
| SL004137 | EphA1 | Ephrin type-A receptor 1 | P21709 | EPHA1 |
| SL004140 | Ephrin-A4 | Ephrin-A4 | P52798 | EFNA4 |
| SL004141 | Ephrin-A5 | Ephrin-A5 | P52803 | EFNA5 |
| SL004142 | Ephrin-B3 | Ephrin-B3 | Q15768 | EFNB3 |
| SL004143 | GFRa-2 | GDNF family receptor alpha-2 | O00451 | GFRA2 |
| SL004144 | GFRa-3 | GDNF family receptor alpha-3 | O60609 | GFRA3 |
| SL004145 | HVEM | Tumor necrosis factor receptor | Q92956 | TNFRSF14 |
| | | superfamily member 14 | | |
| SL004146 | IL-1 R4 | Interleukin-1 receptor-like 1 | Q01638 | IL1RL1 |
| SL004147 | IL-10 Rb | Interleukin-10 receptor subunit beta | Q08334 | IL10RB |
| SL004148 | IL-12 Rb1 | Interleukin-12 receptor subunit beta-1 | P42701 | IL12RB1 |
| SL004149 | IL-13 Ra1 | Interleukin-13 receptor subunit alpha-1 | P78552 | IL13RA1 |
| SL004151 | IL-15 Ra | Interleukin-15 receptor subunit alpha | Q13261 | IL15RA |
| SL004152 | IL-18 Ra | Interleukin-18 receptor 1 | Q13478 | IL18R1 |
| SL004153 | M-CSF R | Macrophage colony-stimulating factor 1 | P07333 | CSF1R |
| | | receptor | | |
| SL004154 | NCAM-L1 | Neural cell adhesion molecule L1 | P32004 | L1CAM |
| SL004155 | PDGF Rb | Platelet-derived growth factor receptor | P09619 | PDGFRB |
| | | beta | | |
| SL004156 | TRAIL R1 | Tumor necrosis factor receptor | O00220 | TNFRSF10A |
| | | superfamily member 10A | | |
| SL004160 | TrkB | BDNF/NT-3 growth factors receptor | Q16620 | NTRK2 |
| SL004180 | CD30 | Tumor necrosis factor receptor | P28908 | TNFRSF8 |
| | | superfamily member 8 | | |
| SL004182 | GV | Calcium-dependent phospholipase A2 | P39877 | PLA2G5 |
| SL004183 | P-Cadherin | Cadherin-3 | P22223 | CDH3 |
| SL004208 | annexin I | Annexin A1 | P04083 | ANXA1 |
| SL004209 | annexin II | Annexin A2 | P07355 | ANXA2 |
| SL004230 | tau | Microtubule-associated protein tau | P10636 | MAPT |
| SL004253 | 17-beta- | Estradiol 17-beta-dehydrogenase 1 | P14061 | HSD17B1 |
| | HSD 1 | | | |
| SL004258 | Adiponectin | Adiponectin | Q15848 | ADIPOQ |
| SL004260 | resistin | Resistin | Q9HD89 | RETN |
| SL004271 | GFAP | Glial fibrillary acidic protein | P14136 | GFAP |
| SL004296 | Myokinase, | Adenylate kinase isoenzyme 1 | P00568 | AK1 |
| | human | | | |
| SL004298 | granzyme A | Granzyme A | P12544 | GZMA |
| SL004299 | Livin B | Baculoviral IAP repeat-containing | Q96CA5 | BIRC7 |
| | | protein 7 I | | |
| SL004301 | Ku70 | X-ray repair cross-complementing | P12956 | XRCC6 |
| | | protein 6 | | |
| SL004304 | STX1a | Syntaxin-1A | Q16623 | STX1A |
| SL004305 | Topoisomerase I | DNA topoisomerase 1 | P11387 | TOP1 |
| SL004306 | UBC9 | SUMO-conjugating enzyme UBC9 | P63279 | UBE2I |
| SL004326 | TNFSF18 | Tumor necrosis factor ligand | Q9UNG2 | TNFSF18 |
| | | superfamily member 18 | | |
| SL004327 | BAFF | Tumor necrosis factor ligand | Q9Y275 | TNFSF13B |
| | | superfamily member 13B | | |
| SL004329 | BMP-14 | Growth/differentiation factor 5 | P43026 | GDF5 |
| SL004330 | CD22 | B-cell receptor CD22 | P20273 | CD22 |
| SL004331 | CNTF | Ciliary Neurotrophic Factor | P26441 | CNTF |
| SL004332 | EG-VEGF | Prokineticin-1 | P58294 | PROK1 |
| SL004333 | FGF-10 | Fibroblast growth factor 10 | O15520 | FGF10 |
| SL004334 | FGF-16 | Fibroblast growth factor 16 | O43320 | FGF16 |
| SL004335 | FGF-17 | Fibroblast growth factor 17 | O60258 | FGF17 |
| SL004336 | FGF-18 | Fibroblast growth factor 18 | O76093 | FGF18 |
| SL004337 | FGF-19 | Fibroblast growth factor 19 | O95750 | FGF19 |
| SL004338 | FGF-20 | Fibroblast growth factor 20 | Q9NP95 | FGF20 |
| SL004339 | FGF-5 | Fibroblast growth factor 5 | P12034 | FGF5 |
| SL004340 | FGF-6 | Fibroblast growth factor 6 | P10767 | FGF6 |
| SL004342 | FGF-8B | Fibroblast growth factor 8 isoform B | P55075 | FGF8 |
| SL004343 | Flt3 ligand | Fms-related tyrosine kinase 3 ligand | P49771 | FLT3LG |
| SL004345 | GDF-11 | Growth/differentiation factor 11 | O95390 | GDF11 |
| SL004346 | IL-20 | Interleukin-20 | Q9NYY1 | IL20 |
| SL004347 | IL-22 | Interleukin-22 | Q9GZX6 | IL22 |
| SL004348 | IFN-lambda 1 | Interferon lambda-1 | Q8IU54 | IFNL1 |
| SL004349 | IFN-lambda 2 | Interferon lambda-2 | Q8IZJ0 | IFNL2 |
| SL004350 | IL-17B | Interleukin-17B | Q9UHF5 | IL17B |
| SL004351 | IL-17E | Interleukin-25 | Q9H293 | IL25 |
| SL004352 | IL-17F | Interleukin-17F | Q96PD4 | IL17F |
| SL004353 | IL-17D | Interleukin-17D | Q8TAD2 | IL17D |
| SL004354 | IL-19 | Interleukin-19 | Q9UHD0 | IL19 |
| SL004355 | LD78-beta | C-C motif chemokine 3-like 1 | P16619 | CCL3L1 |
| SL004356 | LAG-1 | C-C motif chemokine 4-like | Q8NHW4 | CCL4L1 |
| SL004359 | Neurotrophin-3 | Neurotrophin-3 | P20783 | NTF3 |
| SL004360 | Neurotrophin-5 | Neurotrophin-4 | P34130 | NTF4 |
| SL004362 | SCGF-beta | Stem Cell Growth Factor-beta | Q9Y240 | CLEC11A |
| SL004363 | SCGF- | Stem Cell Growth Factor-alpha | Q9Y240 | CLEC11A |
| | alpha | | | |
| SL004364 | TACI | Tumor necrosis factor receptor | O14836 | TNFRSF13B |
| | | superfamily member 13B | | |
| SL004365 | TWEAK | Tumor necrosis factor ligand | O43508 | TNFSF12 |
| | | superfamily member 12 | | |
| SL004366 | TWEAKR | Tumor necrosis factor receptor | Q9NP84 | TNFRSF12A |
| | | superfamily member 12A | | |
| SL004367 | DKK1 | Dickkopf-related protein 1 | O94907 | DKK1 |
| SL004400 | Coagulation | Coagulation factor IXab | P00740 | F9 |
| | Factor IX | | | |
| SL004415 | ACE2 | Angiotensin-converting enzyme 2 | Q9BYF1 | ACE2 |
| SL004438 | Cystatin M | Cystatin-M | Q15828 | CST6 |
| SL004457 | Protease | Glia-derived nexin | P07093 | SERPINE2 |
| | nexin I | | | |
| SL004458 | Elafin | Elafin | P19957 | PI3 |
| SL004466 | Heparin | Heparin cofactor 2 | P05546 | SERPIND1 |
| | cofactor II | | | |
| SL004469 | amyloid | Amyloid beta A4 protein | P05067 | APP |
| | precursor | | | |
| | pro | | | |
| SL004477 | calgranulin B | Protein S100-A9 | P06702 | S100A9 |
| SL004482 | Endoglin | Endoglin | P17813 | ENG |
| SL004484 | SP-D | Pulmonary surfactant-associated protein | P35247 | SFTPD |
| | | D | | |
| SL004486 | VEGF-C | Vascular endothelial growth factor C | P49767 | VEGFC |
| SL004492 | TLR2 | Toll-like receptor 2 | O60603 | TLR2 |
| SL004511 | BPI | Bactericidal permeability-increasing | P17213 | BPI |
| | | protein | | |
| SL004515 | PGRP-S | Peptidoglycan recognition protein 1 | O75594 | PGLYRP1 |
| SL004516 | MBL | Mannose-binding protein C | P11226 | MBL2 |
| SL004536 | LEAP-1 | Hepcidin | P81172 | HAMP |
| SL004556 | DAF | Complement decay-accelerating factor | P08174 | CD55 |
| SL004579 | Macrophage | Macrophage mannose receptor 1 | P22897 | MRC1 |
| | mannose re | | | |
| SL004580 | Macrophage | Macrophage scavenger receptor types I | P21757 | MSR1 |
| | scavenger | and II | | |
| SL004588 | IL-1 R AcP | Interleukin-1 Receptor accessory protein | Q9NPH3 | IL1RAP |
| SL004589 | Azurocidin | Azurocidin | P20160 | AZU1 |
| SL004591 | G-CSF-R | Granulocyte colony-stimulating factor | Q99062 | CSF3R |
| | | receptor | | |
| SL004594 | Troponin I, | Troponin I, fast skeletal muscle | P48788 | TNNI2 |
| | skeletal, | | | |
| SL004605 | 40S | 40S ribosomal protein SA | P08865 | RPSA |
| | ribosomal | | | |
| | protein | | | |
| SL004610 | LRP8 | Low-density lipoprotein receptor-related | Q14114 | LRP8 |
| | | protein 8 | | |
| SL004625 | ADAMTS-4 | A disintegrin and metalloproteinase with | O75173 | ADAMTS4 |
| | | thrombospondin motifs 4 | | |
| SL004626 | ADAMTS-5 | A disintegrin and metalloproteinase with | Q9UNA0 | ADAMTS5 |
| | | thro | | |
| SL004635 | CD30 | Tumor necrosis factor ligand | P32971 | TNFSF8 |
| | Ligand | superfamily member 8 | | |
| SL004636 | Flt-3 | Receptor-type tyrosine-protein kinase | P36888 | FLT3 |
| | | FLT3 | | |
| SL004637 | MSP R | Macrophage-stimulating protein receptor | Q04912 | MST1R |
| SL004639 | TrkC | NT-3 growth factor receptor | Q16288 | NTRK3 |
| SL004642 | ADAM 9 | Disintegrin and metalloproteinase | Q13443 | ADAM9 |
| | | domain-containing protein 9 | | |
| SL004643 | Angiopoietin-4 | Angiopoietin-4 | Q9Y264 | ANGPT4 |
| SL004644 | EDA | Ectodysplasin-A, secreted form | Q92838 | EDA |
| SL004645 | HAI-1 | Kunitz-type protease inhibitor 1 | O43278 | SPINT1 |
| SL004646 | Layilin | Layilin | Q6UX15 | LAYN |
| SL004648 | LIGHT | Tumor necrosis factor ligand | O43557 | TNFSF14 |
| | | superfamily member 14 | | |
| SL004649 | OX40 | Tumor necrosis factor ligand | P23510 | TNFSF4 |
| | Ligand | superfamily member 4 | | |
| SL004650 | sFRP-3 | Secreted frizzled-related protein 3 | Q92765 | FRZB |
| SL004652 | WIF-1 | Wnt inhibitory factor 1 | Q9Y5W5 | WIF1 |
| SL004654 | Granzyme H | Granzyme H | P20718 | GZMH |
| SL004660 | BSP | Bone sialoprotein 2 | P21815 | IBSP |
| SL004661 | Aggrecan | Aggrecan core protein | P16112 | ACAN |
| SL004668 | Apo E3 | Apolipoprotein E (isoform E3) | P02649 | APOE |
| SL004669 | Apo E4 | Apolipoprotein E (isoform E4) | P02649 | APOE |
| SL004670 | Artemin | Artemin | Q5T4W7 | ARTN |
| SL004671 | BAFF | Tumor necrosis factor receptor | Q96RJ3 | TNFRSF13C |
| | Receptor | superfamily member 13C | | |
| SL004672 | BCMA | Tumor necrosis factor receptor | Q02223 | TNFRSF17 |
| | | superfamily member 17 | | |
| SL004673 | Cathepsin S | Cathepsin S | P25774 | CTSS |
| SL004676 | IGFBP-5 | Insulin-like growth factor-binding | P24593 | IGFBP5 |
| | | protein 5 | | |
| SL004683 | Noggin | Noggin | Q13253 | NOG |
| SL004685 | Persephin | Persephin | O60542 | PSPN |
| SL004686 | TNFSF15 | Tumor necrosis factor ligand | O95150 | TNFSF15 |
| | | superfamily member 15 | | |
| SL004687 | TSLP | Thymic stromal lymphopoietin | Q969D9 | TSLP |
| SL004689 | WISP-1 | WNT1-inducible-signaling pathway | O95388 | WISP1 |
| | | protein 1 | | |
| SL004692 | CLF-1/CLC | Cytokine receptor-like factor | O75462 | CRLF1 CLCF1 |
| | Complex | 1:Cardiotrophin | Q9UBD9 | |
| SL004697 | HPV E7 | Protein E7_HPV16 | P03129 | Human-virus |
| | Type 16 | | | |
| SL004698 | HPV E7 | Protein E7_HPV18 | P06788 | Human-virus |
| | Type18 | | | |
| SL004704 | COMMD7 | COMM domain-containing protein 7 | Q86VX2 | COMMD7 |
| SL004708 | CTAP-III | Connective tissue-activating peptide III | P02775 | PPBP |
| SL004712 | SDF-1 | Stromal cell-derived factor 1 | P48061 | CXCL12 |
| SL004714 | LIF sR | Leukemia inhibitory factor receptor | P42702 | LIFR |
| SL004716 | JNK2 | Mitogen-activated protein kinase 9 | P45984 | MAPK9 |
| SL004718 | Karyopherin- | Importin subunit alpha-1 | P52292 | KPNA2 |
| | a2 | | | |
| SL004720 | Calcineurin | Calcineurin subunit B type 1 | P63098 | PPP3R1 |
| | Ba | | | |
| SL004723 | HDAC8 | Histone deacetylase 8 | Q9BY41 | HDAC8 |
| SL004724 | MOZ | Histone acetyltransferase KAT6A | Q92794 | KAT6A |
| SL004725 | Hat1 | Histone acetyltransferase type B | O14929 | HAT1 |
| | | catalytic subunit | | |
| SL004726 | CD97 | CD97 antigen | P48960 | CD97 |
| SL004737 | Tropomyosin | Tropomyosin alpha-1 chain | P09493 | TPM1 |
| | 1 alpha | | | |
| | chain | | | |
| SL004739 | ITI heavy | Inter-alpha-trypsin inhibitor heavy chain | Q14624 | ITIH4 |
| | chain H4 | H4 | | |
| SL004742 | Afamin | Afamin | P43652 | AFM |
| SL004750 | DEAD-box | ATP-dependent RNA helicase DDX19B | Q9UMR2 | DDX19B |
| | protein 19B | | | |
| SL004751 | HO-2 | Heme oxygenase 2 | P30519 | HMOX2 |
| SL004752 | DRR1 | Protein FAM107A | O95990 | FAM107A |
| SL004757 | DRG-1 | Vacuolar protein sorting-associated | Q9NP79 | VTA1 |
| | | protein V | | |
| SL004759 | eIF-5 | Eukaryotic translation initiation factor 5 | P55010 | EIF5 |
| SL004760 | PAFAH | Platelet-activating factor acetylhydrolase | P68402 | PAFAH1B2 |
| | beta subunit | IB | | |
| SL004765 | MAPKAPK3 | MAP kinase-activated protein kinase 3 | Q16644 | MAPKAPK3 |
| SL004768 | AIF1 | Allograft inflammatory factor 1 | P55008 | AIF1 |
| SL004771 | Aurora | Aurora kinase A | O14965 | AURKA |
| | kinase A | | | |
| SL004781 | CSK | Tyrosine-protein kinase CSK | P41240 | CSK |
| SL004782 | TSG-6 | Tumor necrosis factor-inducible gene 6 | P98066 | TNFAIP6 |
| | | protein | | |
| SL004791 | DR3 | Tumor necrosis factor receptor | Q93038 | TNFRSF25 |
| | | superfamily member 25 | | |
| SL004795 | ERAB | 3-hydroxyacyl-CoA dehydrogenase | Q99714 | HSD17B10 |
| | | type-2 | | |
| SL004804 | Nectin-like | Cell adhesion molecule 3 | Q8N126 | CADM3 |
| | protein 1 | | | |
| SL004805 | Nectin-like | Cell adhesion molecule 1 | Q9BY67 | CADM1 |
| | protein 2 | | | |
| SL004812 | Triosephosphate | Triosephosphate isomerase | P60174 | TPI1 |
| | isomese | | | |
| SL004814 | Coactosin- | Coactosin-like protein | Q14019 | COTL1 |
| | like protein | | | |
| SL004820 | Phosphoglycerate | Phosphoglycerate mutase 1 | P18669 | PGAM1 |
| | mutase 1 | | | |
| SL004823 | Cyclophilin A | Peptidyl-prolyl cis-trans isomerase A | P62937 | PPIA |
| SL004837 | Activin AB | Inhibin beta A chain:Inhibin beta B | P08476 | INHBA INHBB |
| | | chain heterodimer | P09529 | |
| SL004844 | EphA5 | Ephrin type-A receptor 5 | P54756 | EPHA5 |
| SL004845 | EphB4 | Ephrin type-B receptor 4 | P54760 | EPHB4 |
| SL004849 | IL-1 sR9 | X-linked interleukin-1 receptor | Q9NP60 | IL1RAPL2 |
| | | accessory protein-like 2 | | |
| SL004850 | IL-17 sR | Interleukin-17 receptor A | Q96F46 | IL17RA |
| SL004851 | ALK-1 | Serine/threonine-protein kinase receptor | P37023 | ACVRL1 |
| | | R3 | | |
| SL004852 | B7-H1 | Programmed cell death 1 ligand 1 | Q9NZQ7 | CD274 |
| SL004853 | B7-H2 | ICOS ligand | O75144 | ICOSLG |
| SL004855 | contactin-1 | Contactin-1 | Q12860 | CNTN1 |
| SL004856 | Desmoglein-1 | Desmoglein-1 | Q02413 | DSG1 |
| SL004857 | Desmoglein-2 | Desmoglein-2 | Q14126 | DSG2 |
| SL004858 | GFRa-1 | GDNF family receptor alpha-1 | P56159 | GFRA1 |
| SL004859 | GITR | Tumor necrosis factor receptor | Q9Y5U5 | TNFRSF18 |
| | | superfamily member 18 | | |
| SL004860 | HTRA2 | Serine protease HTRA2, mitochondrial | O43464 | HTRA2 |
| SL004861 | IL-18 Rb | Interleukin-18 receptor accessory protein | O95256 | IL18RAP |
| SL004862 | PD-L2 | Programmed cell death 1 ligand 2 | Q9BQ51 | PDCD1LG2 |
| SL004863 | TAJ | Tumor necrosis factor receptor | Q9NS68 | TNFRSF19 |
| | | superfamily member 19 | | |
| SL004864 | Cadherin-12 | Cadherin-12 | P55289 | CDH12 |
| SL004865 | Cadherin-6 | Cadherin-6 | P55285 | CDH6 |
| SL004866 | Carbonic | Carbonic anhydrase 1 | P00915 | CA1 |
| | anhydrase I | | | |
| SL004867 | Carbonic | Carbonic anhydrase 3 | P07451 | CA3 |
| | anhydrase II | | | |
| SL004868 | Carbonic | Carbonic anhydrase 7 | P43166 | CA7 |
| | anhydrase VI | | | |
| SL004869 | Carbonic | Carbonic anhydrase 13 | Q8N1Q1 | CA13 |
| | anhydrase XI | | | |
| SL004871 | DR6 | Tumor necrosis factor receptor | O75509 | TNFRSF21 |
| | | superfamily member 21 | | |
| SL004872 | EDAR | Tumor necrosis factor receptor | Q9UNE0 | EDAR |
| | | superfamily member EDAR | | |
| SL004875 | IL-1Rrp2 | Interleukin-1 receptor-like 2 | Q9HB29 | IL1RL2 |
| SL004876 | Kallistatin | Kallistatin | P29622 | SERPINA4 |
| SL004891 | hnRNP | Heterogeneous nuclear | P22626 | HNRNPA2B1 |
| | A2/B1 | ribonucleoproteins A2/B | | |
| SL004899 | HSP70 | Heat shock cognate 71 kDa protein | P11142 | HSPA8 |
| | protein 8 | | | |
| SL004901 | Protein | Protein disulfide-isomerase | P07237 | P4HB |
| | disulfide- | | | |
| | isomerase | | | |
| SL004908 | Tropomyosin 2 | Tropomyosin beta chain | P07951 | TPM2 |
| SL004914 | PPase | Inorganic pyrophosphatase | Q15181 | PPA1 |
| SL004915 | NCC27 | Chloride intracellular channel protein 1 | O00299 | CLIC1 |
| SL004919 | Peroxiredoxin-1 | Peroxiredoxin-1 | Q06830 | PRDX1 |
| SL004920 | Cofilin-1 | Cofilin-1 | P23528 | CFL1 |
| SL004921 | NDP kinase B | Nucleoside diphosphate kinase B | P22392 | NME2 |
| SL004925 | AGR2 | Anterior gradient protein 2 homolog | O95994 | AGR2 |
| SL004932 | Peroxiredoxin-5 | Peroxiredoxin-5, mitochondrial | P30044 | PRDX5 |
| SL004938 | CaMKK | Calcium/calmodulin-dependent protein | Q8N5S9 | CAMKK1 |
| | alpha | kinase k | | |
| SL004939 | PTP-1B | Tyrosine-protein phosphatase non- | P18031 | PTPN1 |
| | | receptor type 1B | | |
| SL004940 | PTP-1C | Tyrosine-protein phosphatase non- | P29350 | PTPN6 |
| | | receptor type 1C | | |
| SL005034 | RAN | GTP-binding nuclear protein Ran | P62826 | RAN |
| SL005059 | TGF-b R III | Transforming growth factor beta | Q03167 | TGFBR3 |
| | | receptor type | | |
| SL005084 | Periostin | Periostin | Q15063 | POSTN |
| SL005087 | IGFBP-7 | Insulin-like growth factor-binding | Q16270 | IGFBP7 |
| | | protein 7 | | |
| SL005102 | SHBG | Sex hormone-binding globulin | P04278 | SHBG |
| SL005115 | Spondin-1 | Spondin-1 | Q9HCB6 | SPON1 |
| SL005152 | TIG2 | Retinoic acid receptor responder protein | Q99969 | RARRES2 |
| | | 2 | | |
| SL005153 | CNTFR | Ciliary neurotrophic factor receptor | P26992 | CNTFR |
| | alpha | subunit | | |
| SL005155 | Cripto | Teratocarcinoma-derived growth factor 1 | P13385 | TDGF1 |
| SL005156 | DAN | Neuroblastoma suppressor of | P41271 | NBL1 |
| | | tumorigenicity 1 | | |
| SL005157 | DC-SIGN | CD209 antigen | Q9NNX6 | CD209 |
| SL005158 | DC-SIGNR | C-type lectin domain family 4 member | Q9H2X3 | CLEC4M |
| | | M | | |
| SL005159 | EPO-R | Erythropoietin receptor | P19235 | EPOR |
| SL005160 | ESAM | Endothelial cell-selective adhesion | Q96AP7 | ESAM |
| | | molecule | | |
| SL005161 | FGF-12 | Fibroblast growth factor 12 | P61328 | FGF12 |
| SL005164 | Galectin-2 | Galectin-2 | P05162 | LGALS2 |
| SL005165 | Galectin-4 | Galectin-4 | P56470 | LGALS4 |
| SL005167 | Galectin-8 | Galectin-8 | O00214 | LGALS8 |
| SL005168 | Growth | Growth hormone receptor | P10912 | GHR |
| | hormone | | | |
| | receptor | | | |
| SL005169 | sICAM-5 | Intercellular adhesion molecule 5 | Q9UMF0 | ICAM5 |
| SL005170 | ICOS | Inducible T-cell costimulator | Q9Y6W8 | ICOS |
| SL005171 | IGFBP-4 | Insulin-like growth factor-binding | P22692 | IGFBP4 |
| | | protein 4 | | |
| SL005172 | IGFBP-6 | Insulin-like growth factor-binding | P24592 | IGFBP6 |
| | | protein 6 | | |
| SL005174 | IL-17B R | interleukin-17 receptor B | Q9NRM6 | IL17RB |
| SL005178 | IL-1F7 | Interleukin-37 | Q9NZH6 | IL37 |
| SL005181 | IL-20 Ra | Interleukin-20 receptor subunit alpha | Q9UHF4 | IL20RA |
| SL005183 | IL-22BP | Interleukin-22 receptor subunit alpha-2 | Q969J5 | IL22RA2 |
| SL005184 | IL-23 | Interleukin-23 | P29460, | IL12B IL23A |
| | | | Q9NPF7 | |
| SL005185 | IL-23 R | Interleukin-23 receptor | Q5VWK5 | IL23R |
| SL005187 | IL-3 Ra | Interleukin-3 receptor subunit alpha | P26951 | IL3RA |
| SL005188 | IL-5 Ra | Interleukin-5 receptor subunit alpha | Q01344 | IL5RA |
| SL005189 | IL-7 Ra | Interleukin-7 receptor subunit alpha | P16871 | IL7R |
| SL005190 | ILT-2 | Leukocyte immunoglobulin-like receptor | Q8NHL6 | LILRB1 |
| | | subfamily B member 1 | | |
| SL005191 | ILT-4 | Leukocyte immunoglobulin-like receptor | Q8N423 | LILRB2 |
| | | subfamily B member 2 | | |
| SL005193 | JAM-B | Junctional adhesion molecule B | P57087 | JAM2 |
| SL005194 | JAM-C | Junctional adhesion molecule C | Q9BX67 | JAM3 |
| SL005195 | LAG-3 | Lymphocyte activation gene 3 protein | P18627 | LAG3 |
| SL005196 | LSAMP | Limbic system-associated membrane | Q13449 | LSAMP |
| | | protein | | |
| SL005197 | LIMP II | Lysosome membrane protein 2 | Q14108 | SCARB2 |
| SL005199 | MICA | MHC class I polypeptide-related | Q29983 | MICA |
| | | sequence A | | |
| SL005200 | MICB | MHC class I polypeptide-related | Q29980 | MICB |
| | | sequence B | | |
| SL005201 | MIS | Muellerian-inhibiting factor | P03971 | AMH |
| SL005202 | MSP | Hepatocyte growth factor-like protein | P26927 | MST1 |
| SL005204 | NKG2D | NKG2-D type II integral membrane | P26718 | KLRK1 |
| | | protein | | |
| SL005205 | NKp30 | Natural cytotoxicity triggering receptor 3 | O14931 | NCR3 |
| SL005206 | NKp44 | Natural cytotoxicity triggering receptor 2 | O95944 | NCR2 |
| SL005207 | NKp46 | Natural cytotoxicity triggering receptor 1 | O76036 | NCR1 |
| SL005208 | Nogo | Reticulon-4 receptor | Q9BZR6 | RTN4R |
| | Receptor | | | |
| SL005209 | Notch-3 | Neurogenic locus notch homolog protein | Q9UM47 | NOTCH3 |
| | | 3 | | |
| SL005210 | Nr-CAM | Neuronal cell adhesion molecule | Q92823 | NRCAM |
| SL005212 | Prolactin | Prolactin receptor | P16471 | PRLR |
| | Receptor | | | |
| SL005213 | RELT | Tumor necrosis factor receptor | Q969Z4 | RELT |
| | | superfamily member 19L | | |
| SL005214 | Semaphorin- | Semaphorin-6A | Q9H2E6 | SEMA6A |
| | 6A | | | |
| SL005215 | Siglec-3 | Myeloid cell surface antigen CD33 | P20138 | CD33 |
| SL005217 | Siglec-6 | Sialic acid-binding Ig-like lectin 6 | O43699 | SIGLEC6 |
| SL005218 | Siglec-7 | Sialic acid-binding Ig-like lectin 7 | Q9Y286 | SIGLEC7 |
| SL005219 | Siglec-9 | Sialic acid-binding Ig-like lectin 9 | Q9Y336 | SIGLEC9 |
| SL005220 | Sonic | Sonic hedgehog protein | Q15465 | SHH |
| | Hedgehog | | | |
| SL005221 | SREC-I | Scavenger receptor class F member 1 | Q14162 | SCARF1 |
| SL005222 | SREC-II | Scavenger receptor class F member 2 | Q96GP6 | SCARF2 |
| SL005223 | TCCR | Interleukin-27 receptor subunit alpha | Q6UWB1 | IL27RA |
| SL005224 | Thrombopoietin | Thrombopoietin Receptor | P40238 | MPL |
| | Receptor | | | |
| SL005225 | TrkA | High affinity nerve growth factor | P04629 | NTRK1 |
| | | receptor | | |
| SL005226 | TSLP R | Cytokine receptor-like factor 2 | Q9HC73 | CRLF2 |
| SL005227 | ULBP-1 | NKG2D ligand 1 | Q9BZM6 | ULBP1 |
| SL005228 | ULBP-2 | NKG2D ligand 2 | Q9BZM5 | ULBP2 |
| SL005229 | ULBP-3 | NKG2D ligand 3 | Q9BZM4 | ULBP3 |
| SL005230 | UNC5H3 | Netrin receptor UNC5C | O95185 | UNC5C |
| SL005231 | UNC5H4 | Netrin receptor UNC5D | Q6UXZ4 | UNC5D |
| SL005233 | XEDAR | Tumor necrosis factor receptor | Q9HAV5 | EDA2R |
| | | superfamily member 27 | | |
| SL005234 | GDF-9 | Growth/differentiation factor 9 | O60383 | GDF9 |
| SL005235 | NANOG | Homeobox protein NANOG | Q9H9S0 | NANOG |
| SL005236 | NovH | Protein NOV homolog | P48745 | NOV |
| SL005250 | Chymase | Chymase | P23946 | CMA1 |
| SL005256 | Histone | Histone H1.2 | P16403 | HIST1H1C |
| | H1.2 | | | |
| SL005258 | PLK-1 | Serine/threonine-protein kinase PLK1 | P53350 | PLK1 |
| SL005261 | TCPTP | Tyrosine-protein phosphatase non- | P17706 | PTPN2 |
| | | receptor type 2 | | |
| SL005263 | RAP | alpha-2-macroglobulin receptor- | P30533 | LRPAP1 |
| | | associated protein | | |
| SL005308 | PSME3 | Proteasome activator complex subunit 3 | P61289 | PSME3 |
| SL005352 | FABPE | Fatty acid-binding protein, epidermal | Q01469 | FABP5 |
| SL005358 | prostatic | Phosphatidylethanolamine-binding | P30086 | PEBP1 |
| | binding pro | protein 1 | | |
| SL005361 | Apo D | Apolipoprotein D | P05090 | APOD |
| SL005372 | Sorting | Sorting nexin-4 | O95219 | SNX4 |
| | nexin 4 | | | |
| SL005392 | Arylsulfatase A | Arylsulfatase A | P15289 | ARSA |
| SL005437 | MEPE | Matrix extracellular | Q9NQ76 | MEPE |
| | | phosphoglycoprotein | | |
| SL005488 | SPARCL1 | SPARC-like protein 1 | Q14515 | SPARCL1 |
| SL005491 | OBCAM | Opioid-binding protein/cell adhesion | Q14982 | OPCML |
| | | molecule | | |
| SL005493 | paraoxonase 1 | Serum paraoxonase/arylesterase 1 | P27169 | PON1 |
| SL005508 | Carbonic | Carbonic anhydrase 9 | Q16790 | CA9 |
| | anhydrase 9 | | | |
| SL005572 | Gelsolin | Gelsolin | P06396 | GSN |
| SL005574 | Aminoacylase-1 | Aminoacylase-1 | Q03154 | ACY1 |
| SL005575 | Fucosyltrans- | Galactoside 3(4)-L-fucosyltransferase | P21217 | FUT3 |
| | ferase 3 | | | |
| SL005588 | FER | Tyrosine-protein kinase Fer | P16591 | FER |
| SL005629 | NAGK | N-acetyl-D-glucosamine kinase | Q9UJ70 | NAGK |
| SL005630 | PSA6 | Proteasome subunit alpha type-6 | P60900 | PSMA6 |
| SL005675 | ATP | ATP synthase subunit beta, | P06576 | ATP5B |
| | synthase | mitochondrial | | |
| | beta cha | | | |
| SL005679 | TCTP | Translationally-controlled tumor protein | P13693 | TPT1 |
| SL005685 | EF-1-beta | Elongation factor 1-beta | P24534 | EEF1B2 |
| SL005687 | eIF-5A-1 | Eukaryotic translation initiation factor | P63241 | EIF5A |
| | | 5A-1 | | |
| SL005694 | Peroxiredoxin-6 | Peroxiredoxin-6 | P30041 | PRDX6 |
| SL005703 | Notch 1 | Neurogenic locus notch homolog protein | P46531 | NOTCH1 |
| | | 1 | | |
| SL005725 | GRB2- | GRB2-related adapter protein 2 | O75791 | GRAP2 |
| | related | | | |
| | adapter | | | |
| SL005730 | cGMP- | cGMP-dependent 3′,5′-cyclic | O00408 | PDE2A |
| | stimulated | phosphodiesterase | | |
| | PDE | | | |
| SL005764 | sCD163 | Scavenger receptor cysteine-rich type 1 | Q86VB7 | CD163 |
| | | protein M130 | | |
| SL005789 | Stanniocalcin-1 | Stanniocalcin-1 | P52823 | STC1 |
| SL005793 | Cyclophilin F | Peptidyl-prolyl cis-trans isomerase F, | P30405 | PPIF |
| | | mitochondrial | | |
| SL005797 | PIGR | Polymeric immunoglobulin receptor | P01833 | PIGR |
| SL005846 | Moesin | Moesin | P26038 | MSN |
| SL006029 | Chitotriosidase-1 | Chitotriosidase-1 | Q13231 | CHIT1 |
| SL006088 | Sphingosine | Sphingosine kinase 1 | Q9NYA1 | SPHK1 |
| | kinase 1 | | | |
| SL006091 | NCK1 | Cytoplasmic protein NCK1 | P16333 | NCK1 |
| SL006108 | CD5L | CD5 antigen-like | O43866 | CD5L |
| SL006114 | ROR1 | Tyrosine-protein kinase transmembrane | Q01973 | ROR1 |
| | | receptor ROR1 | | |
| SL006119 | TFF3 | Trefoil factor 3 | Q07654 | TFF3 |
| SL006132 | Lamin-B1 | Lamin-B1 | P20700 | LMNB1 |
| SL006189 | KIF23 | Kinesin-like protein KIF23 | Q02241 | KIF23 |
| SL006197 | DnaJ | Mitochondrial import inner membrane | Q96DA6 | DNAJC19 |
| | homolog | translocase subunit TIM14 | | |
| SL006268 | NSF1C | NSFL1 cofactor p47 | Q9UNZ2 | NSFL1C |
| SL006372 | YES | Tyrosine-protein kinase Yes | P07947 | YES1 |
| SL006374 | BMX | Cytoplasmic tyrosine-protein kinase | P51813 | BMX |
| | | BMX | | |
| SL006378 | Esterase D | S-formylglutathione hydrolase | P10768 | ESD |
| SL006397 | NRP1 | Neuropilin-1 | O14786 | NRP1 |
| SL006406 | PLXC1 | Plexin-C1 | O60486 | PLXNC1 |
| SL006448 | HRG | Histidine-rich glycoprotein | P04196 | HRG |
| SL006460 | GP1BA | Platelet glycoprotein Ib alpha chain | P07359 | GP1BA |
| SL006476 | NMT1 | Glycylpeptide N- | P30419 | NMT1 |
| | | tetradecanoyltransferase 1 | | |
| SL006480 | TRY3 | Trypsin-3 | P35030 | PRSS3 |
| SL006512 | HGFA | Hepatocyte growth factor activator | Q04756 | HGFAC |
| SL006522 | LG3BP | Galectin-3-binding protein | Q08380 | LGALS3BP |
| SL006523 | MFGM | Lactadherin | Q08431 | MFGE8 |
| SL006528 | SEPR | Seprase | Q12884 | FAP |
| SL006542 | FCN2 | Ficolin-2 | Q15485 | FCN2 |
| SL006544 | BGH3 | Transforming growth factor-beta- | Q15582 | TGFBI |
| | | induced protein ig-h3 | | |
| SL006550 | ECM1 | Extracellular matrix protein 1 | Q16610 | ECM1 |
| SL006610 | ATS13 | A disintegrin and metalloproteinase with | Q76LX8 | ADAMTS13 |
| | | thro | | |
| SL006629 | SIRT2 | NAD-dependent protein deacetylase | Q8IXJ6 | SIRT2 |
| | | sirtuin-2 | | |
| SL006675 | CKAP2 | Cytoskeleton-associated protein 2 | Q8WWK9 | CKAP2 |
| SL006694 | CNDP1 | Beta-Ala-His dipeptidase | Q96KN2 | CNDP1 |
| SL006698 | transcription | Ligand-dependent nuclear receptor | Q8N3X6 | LCORL |
| | factor | corepressor | | |
| SL006705 | PFD5 | Prefoldin subunit 5 | Q99471 | PFDN5 |
| SL006713 | Collectin | Collectin-11 | Q9BWP8 | COLEC11 |
| | Kidney 1 | | | |
| SL006777 | FETUB | Fetuin-B | Q9UGM5 | FETUB |
| SL006803 | ANGL3 | Angiopoietin-related protein 3 | Q9Y5C1 | ANGPTL3 |
| SL006805 | MRCKB | Serine/threonine-protein kinase MRCK | Q9Y5S2 | CDC42BPB |
| | | beta | | |
| SL006830 | complement | Complement factor H-related protein 5 | Q9BXR6 | CFHR5 |
| | factor H-r | | | |
| SL006892 | ABL1 | Tyrosine-protein kinase ABL1 | P00519 | ABL1 |
| SL006910 | Cathepsin V | Cathepsin L2 | O60911 | CTSV |
| SL006911 | CHK1 | Serine/threonine-protein kinase Chk1 | O14757 | CHEK1 |
| SL006912 | FGR | Tyrosine-protein kinase Fgr | P09769 | FGR |
| SL006913 | FYN | Tyrosine-protein kinase Fyn | P06241 | FYN |
| SL006914 | Glucocorticoid | Glucocorticoid receptor | P04150 | NR3C1 |
| | receptor | | | |
| SL006915 | IL-27 | Interleukin-27 | Q8NEV9 | IL27 EBI3 |
| | | | Q14213 | |
| SL006916 | LCK | Tyrosine-protein kinase Lck | P06239 | LCK |
| SL006917 | LYN | Tyrosine-protein kinase Lyn | P07948 | LYN |
| SL006918 | MK01 | Mitogen-activated protein kinase 1 | P28482 | MAPK1 |
| SL006919 | RSK-like | Ribosomal protein S6 kinase alpha-5 | O75582 | RPS6KA5 |
| | protein | | | |
| | kinase | | | |
| SL006920 | MAPK14 | Mitogen-activated protein kinase 14 | Q16539 | MAPK14 |
| SL006921 | PDK1 | [Pyruvate dehydrogenase (acetyl- | Q15118 | PDK1 |
| | | transferring) | | |
| SL006922 | RAD51 | DNA repair protein RAD51 homolog 1 | Q06609 | RAD51 |
| SL006923 | TBP | TATA-box-binding protein | P20226 | TBP |
| SL006924 | ART | Agouti-related protein | O00253 | AGRP |
| SL006970 | DLL1 | Delta-like protein 1 | O00548 | DLL1 |
| SL006992 | MATN3 | Matrilin-3 | O15232 | MATN3 |
| SL006993 | MK13 | Mitogen-activated protein kinase 13 | O15264 | MAPK13 |
| SL006998 | PDPK1 | 3-phosphoinositide-dependent protein | O15530 | PDPK1 |
| | | kinase 1 | | |
| SL007003 | DHH | Desert hedgehog protein N-product | O43323 | DHH |
| SL007022 | HNRPQ | Heterogeneous nuclear | O60506 | SYNCRIP |
| | | ribonucleoprotein Q | | |
| SL007024 | GREM1 | Gremlin-1 | O60565 | GREM1 |
| SL007025 | JAK2 | Tyrosine-protein kinase JAK2 | O60674 | JAK2 |
| SL007049 | CYTF | Cystatin-F | O76096 | CST7 |
| SL007056 | BMP10 | Bone morphogenetic protein 10 | O95393 | BMP10 |
| SL007059 | LY86 | Lymphocyte antigen 86 | O95711 | LY86 |
| SL007100 | LKHA4 | Leukotriene A-4 hydrolase | P09960 | LTA4H |
| SL007121 | CATE | Cathepsin E | P14091 | CTSE |
| SL007122 | IDE | Insulin-degrading enzyme | P14735 | IDE |
| SL007145 | NR1D1 | Nuclear receptor subfamily 1 group D | P20393 | NR1D1 |
| | | member 1 | | |
| SL007153 | PERL | Lactoperoxidase | P22079 | LPO |
| SL007173 | GRN | Granulins | P28799 | GRN |
| SL007179 | EPHB2 | Ephrin type-B receptor 2 | P29323 | EPHB2 |
| SL007181 | TYK2 | Non-receptor tyrosine-protein kinase | P29597 | TYK2 |
| | | TYK2 | | |
| SL007195 | CD70 | CD70 antigen | P32970 | CD70 |
| SL007206 | TSP2 | Thrombospondin-2 | P35442 | THBS2 |
| SL007207 | TSP4 | Thrombospondin-4 | P35443 | THBS4 |
| SL007228 | KPCI | Protein kinase C iota type | P41743 | PRKCI |
| SL007237 | MP2K4 | Dual specificity mitogen-activated | P45985 | MAP2K4 |
| | | protein kinase | | |
| SL007250 | PK3CG | Phosphatidylinositol 4,5-bisphosphate 3- | P48736 | PIK3CG |
| | | kinase | | |
| SL007261 | AMPM2 | Methionine aminopeptidase 2 | P50579 | METAP2 |
| SL007266 | PSD7 | 26S proteasome non-ATPase regulatory | P51665 | PSMD7 |
| | | subunit | | |
| SL007280 | CATC | Dipeptidyl peptidase 1 | P53634 | CTSC |
| SL007281 | MK12 | Mitogen-activated protein kinase 12 | P53778 | MAPK12 |
| SL007284 | CRIS3 | Cysteine-rich secretory protein 3 | P54108 | CRISP3 |
| SL007295 | CAD15 | Cadherin-15 | P55291 | CDH15 |
| SL007324 | CSK21 | Casein kinase II subunit alpha | P68400 | CSNK2A1 |
| SL007327 | OLR1 | Oxidized low-density lipoprotein | P78380 | OLR1 |
| | | receptor 1 | | |
| SL007328 | JAG1 | Protein jagged-1 | P78504 | JAG1 |
| SL007336 | SET | Protein SET | Q01105 | SET |
| SL007356 | NOTC2 | Neurogenic locus notch homolog protein | Q04721 | NOTCH2 |
| | | 2 | | |
| SL007358 | KPCT | Protein kinase C theta type | Q04759 | PRKCQ |
| SL007373 | PPID | Peptidyl-prolyl cis-trans isomerase D | Q08752 | PPID |
| SL007385 | IL24 | Interleukin-24 | Q13007 | IL24 |
| SL007403 | DMP1 | Dentin matrix acidic phosphoprotein 1 | Q13316 | DMP1 |
| SL007423 | IL-11 RA | Interleukin-11 receptor subunit alpha | Q14626 | IL11RA |
| SL007429 | GPNMB | Transmembrane glycoprotein NMB | Q14956 | GPNMB |
| SL007453 | MK11 | Mitogen-activated protein kinase 11 | Q15759 | MAPK11 |
| SL007464 | AMHR2 | Anti-Muellerian hormone type-2 | Q16671 | AMHR2 |
| | | receptor | | |
| SL007471 | COLEC12 | Collectin-12 | Q5KU26 | COLEC12 |
| SL007502 | ST4S6 | Carbohydrate sulfotransferase 15 | Q7LFX5 | CHST15 |
| SL007531 | BMPER | BMP-binding endothelial regulator | Q8N8U9 | BMPER |
| | | protein | | |
| SL007547 | TIMD3 | Hepatitis A virus cellular receptor 2 | Q8TDQ0 | HAVCR2 |
| SL007560 | STAB2 | Stabilin-2 | Q8WWQ8 | STAB2 |
| SL007620 | IL-12 RB2 | Interleukin-12 receptor subunit beta-2 | Q99665 | IL12RB2 |
| SL007640 | CLC7A | C-type lectin domain family 7 member | Q9BXN2 | CLEC7A |
| | | A | | |
| SL007642 | ANGL4 | Angiopoietin-related protein 4 | Q9BY76 | ANGPTL4 |
| SL007651 | FGF23 | Fibroblast growth factor 23 | Q9GZV9 | FGF23 |
| SL007673 | NET4 | Netrin-4 | Q9HB63 | NTN4 |
| SL007674 | LY9 | T-lymphocyte surface antigen Ly-9 | Q9HBG7 | LY9 |
| SL007680 | ROBO2 | Roundabout homolog 2 | Q9HCK4 | ROBO2 |
| SL007729 | ARTS1 | Endoplasmic reticulum aminopeptidase | Q9NZ08 | ERAP1 |
| | | 1 | | |
| SL007747 | TBK1 | Serine/threonine-protein kinase TBK1 | Q9UHD2 | TBK1 |
| SL007752 | DAPK2 | Death-associated protein kinase 2 | Q9UIK4 | DAPK2 |
| SL007756 | GDF2 | Growth/differentiation factor 2 | Q9UK05 | GDF2 |
| SL007774 | JAG2 | Protein jagged-2 | Q9Y219 | JAG2 |
| SL007804 | BGN | Biglycan | P21810 | BGN |
| SL007806 | IL22RA1 | Interleukin-22 receptor subunit alpha-1 | Q8N6P7 | IL22RA1 |
| SL007869 | PPIB | Peptidyl-prolyl cis-trans isomerase B | P23284 | PPIB |
| SL007871 | Cytidylate | UMP-CMP kinase | P30085 | CMPK1 |
| | kinase | | | |
| SL007888 | Cystatin-S | Cystatin-S | P01036 | CST4 |
| SL008008 | ARGI1 | Arginase-1 | P05089 | ARG1 |
| SL008023 | HPLN1 | Hyaluronan and proteoglycan link | P10915 | HAPLN1 |
| | | protein 1 | | |
| SL008039 | AK1A1 | Alcohol dehydrogenase [NADP(+)] | P14550 | AKR1A1 |
| SL008059 | RS3 | 40S ribosomal protein S3 | P23396 | RPS3 |
| SL008063 | PPAC | Low molecular weight phosphotyrosine | P24666 | ACP1 |
| | | protein | | |
| SL008072 | CO8A1 | Collagen alpha-1(VIII) chain | P27658 | COL8A1 |
| SL008085 | 3HIDH | 3-hydroxyisobutyrate dehydrogenase, | P31937 | HIBADH |
| | | mitochondrial | | |
| SL008099 | CAPG | Macrophage-capping protein | P40121 | CAPG |
| SL008102 | MDHC | Malate dehydrogenase, cytoplasmic | P40925 | MDH1 |
| SL008122 | DUS3 | Dual specificity protein phosphatase 3 | P51452 | DUSP3 |
| SL008143 | UBE2N | Ubiquitin-conjugating enzyme E2 N | P61088 | UBE2N |
| SL008157 | UB2L3 | Ubiquitin-conjugating enzyme E2 L3 | P68036 | UBE2L3 |
| SL008176 | PSME1 | Proteasome activator complex subunit 1 | Q06323 | PSME1 |
| SL008177 | C1QBP | Complement component 1 Q | Q07021 | C1QBP |
| | | subcomponent-binding | | |
| SL008178 | DERM | Dermatopontin | Q07507 | DPT |
| SL008190 | SPTA2 | Spectrin alpha chain, non-erythrocytic 1 | Q13813 | SPTAN1 |
| SL008193 | NID2 | Nidogen-2 | Q14112 | NID2 |
| SL008309 | RTN4 | Reticulon-4 | Q9NQC3 | RTN4 |
| SL008331 | PA2G4 | Proliferation-associated protein 2G4 | Q9UQ80 | PA2G4 |
| SL008378 | 4EBP2 | Eukaryotic translation initiation factor | Q13542 | EIF4EBP2 |
| | | 4E-b | | |
| SL008380 | CATZ | Cathepsin Z | Q9UBR2 | CTSZ |
| SL008382 | CYTD | Cystatin-D | P28325 | CST5 |
| SL008414 | EphB6 | Ephrin type-B receptor 6 | O15197 | EPHB6 |
| SL008416 | MRC2 | C-type mannose receptor 2 | Q9UBG0 | MRC2 |
| SL008421 | ATS1 | A disintegrin and metalloproteinase with | Q9UHI8 | ADAMTS1 |
| | | thrombospondin motifs 1 | | |
| SL008504 | GNS | N-acetylglucosamine-6-sulfatase | P15586 | GNS |
| SL008516 | CYTT | Cystatin-SA | P09228 | CST2 |
| SL008574 | OMD | Osteomodulin | Q99983 | OMD |
| SL008588 | SLAF5 | SLAM family member 5 | Q9UIB8 | CD84 |
| SL008590 | Olfactomedin-4 | Olfactomedin-4 | Q6UX06 | OLFM4 |
| SL008609 | FCG3B | Low affinity immunoglobulin gamma Fc | O75015 | FCGR3B |
| | | region r | | |
| SL008611 | ASAHL | N-acylethanolamine-hydrolyzing acid | Q02083 | NAAA |
| | | amidase | | |
| SL008623 | CNTN2 | Contactin-2 | Q02246 | CNTN2 |
| SL008639 | IDS | Iduronate 2-sulfatase | P22304 | IDS |
| SL008644 | BST1 | ADP-ribosyl cyclase/cyclic ADP-ribose | Q10588 | BST1 |
| | | hydrolase | | |
| SL008703 | CBPE | Carboxypeptidase E | P16870 | CPE |
| SL008709 | DSC3 | Desmocollin-3 | Q14574 | DSC3 |
| SL008728 | NRX3B | Neurexin-3-beta | Q9HDB5 | NRXN3 |
| SL008759 | GPVI | Platelet glycoprotein VI | Q9HCN6 | GP6 |
| SL008773 | CD109 | CD109 antigen | Q6YHK3 | CD109 |
| SL008808 | SKP1 | S-phase kinase-associated protein 1 | P63208 | SKP1 |
| SL008822 | EMR2 | EGF-like module-containing mucin-like | Q9UHX3 | EMR2 |
| | | hormone | | |
| SL008835 | ASGR1 | Asialoglycoprotein receptor 1 | P07306 | ASGR1 |
| SL008865 | PSA2 | Proteasome subunit alpha type-2 | P25787 | PSMA2 |
| SL008904 | LYVE1 | Lymphatic vessel endothelial hyaluronic | Q9Y5Y7 | LYVE1 |
| | | acid | | |
| SL008909 | LGMN | Legumain | Q99538 | LGMN |
| SL008916 | DPP2 | Dipeptidyl peptidase 2 | Q9UHL4 | DPP7 |
| SL008933 | PARK7 | Protein DJ-1 | Q99497 | PARK7 |
| SL008936 | CHL1 | Neural cell adhesion molecule L1-like | O00533 | CHL1 |
| | | protein | | |
| SL008945 | TGM3 | Protein-glutamine gamma- | Q08188 | TGM3 |
| | | glutamyltransferase E | | |
| SL008956 | ARSB | Arylsulfatase B | P15848 | ARSB |
| SL009045 | ENPP7 | Ectonucleotide | Q6UWV6 | ENPP7 |
| | | pyrophosphatase/phosphodiester | | |
| SL009054 | NRX1B | Neurexin-1-beta | P58400 | NRXN1 |
| SL009089 | PGCB | Brevican core protein | Q96GW7 | BCAN |
| SL009202 | JAML1 | Junctional adhesion molecule-like | Q86YT9 | AMICA1 |
| SL009207 | Dynactin | Dynactin subunit 2 | Q13561 | DCTN2 |
| | subunit 2 | | | |
| SL009213 | Cathepsin A | Lysosomal protective protein | P10619 | CTSA |
| SL009216 | dopa | Aromatic-L-amino-acid decarboxylase | P20711 | DDC |
| | decarboxylase | | | |
| SL009324 | FSTL3 | Follistatin-related protein 3 | O95633 | FSTL3 |
| SL009341 | BASI | Basigin | P35613 | BSG |
| SL009400 | CRDL1 | Chordin-like protein 1 | Q9BU40 | CHRDL1 |
| SL009412 | DKK3 | Dickkopf-related protein 3 | Q9UBP4 | DKK3 |
| SL009431 | HINT1 | Histidine triad nucleotide-binding | P49773 | HINT1 |
| | | protein 1 | | |
| SL009628 | ING1 | Inhibitor of growth protein 1 | Q9UK53 | ING1 |
| SL009629 | MBD4 | Methyl-CpG-binding domain protein 4 | O95243 | MBD4 |
| SL009768 | CBX5 | Chromobox protein homolog 5 | P45973 | CBX5 |
| SL009790 | RUXF | Small nuclear ribonucleoprotein F | P62306 | SNRPF |
| SL009791 | hnRNP A/B | Heterogeneous nuclear | Q99729 | HNRNPAB |
| | | ribonucleoprotein A/B | | |
| SL009792 | PUR8 | Adenylosuccinate lyase | P30566 | ADSL |
| SL009868 | SSRP1 | FACT complex subunit SSRP1 | Q08945 | SSRP1 |
| SL009951 | WNT7A | Protein Wnt-7a | O00755 | WNT7A |
| SL009988 | ADAM12 | Disintegrin and metalloproteinase | O43184 | ADAM12 |
| | | domain-containing protein 12 | | |
| SL010250 | Stress- | Stress-induced-phosphoprotein 1 | P31948 | STIP1 |
| | induced- | | | |
| | phosph | | | |
| SL010288 | Carbonic | Carbonic anhydrase 6 | P23280 | CA6 |
| | anhydrase 6 | | | |
| SL010328 | MED-1 | Mediator of RNA polymerase II | Q15648 | MED1 |
| | | transcription subunit 1 | | |
| SL010348 | FN1.4 | Fibronectin Fragment 4 | P02751 | FN1 |
| SL010349 | FN1.3 | Fibronectin Fragment 3 | P02751 | FN1 |
| SL010368 | IDUA | alpha-L-iduronidase | P35475 | IDUA |
| SL010369 | Carbonic | Carbonic anhydrase 4 | P22748 | CA4 |
| | Anhydrase | | | |
| | IV | | | |
| SL010371 | CD39 | Ectonucleoside triphosphate | P49961 | ENTPD1 |
| | | diphosphohydrolase | | |
| SL010372 | Enterokinase | Enteropeptidase | P98073 | TMPRSS15 |
| SL010373 | FCAR | Immunoglobulin alpha Fc receptor | P24071 | FCAR |
| SL010374 | METAP1 | Methionine aminopeptidase 1 | P53582 | METAP1 |
| SL010375 | ASAH2 | Neutral ceramidase | Q9NR71 | ASAH2 |
| SL010376 | MMEL2 | Membrane metallo-endopeptidase-like 1 | Q495T6 | MMEL1 |
| SL010378 | RET | Proto-oncogene tyrosine-protein kinase | P07949 | RET |
| | | receptor | | |
| SL010379 | Semaphorin | Semaphorin-3A | Q14563 | SEMA3A |
| | 3A | | | |
| SL010381 | Soggy-1 | Dickkopf-like protein 1 | Q9UK85 | DKKL1 |
| SL010384 | Testican-1 | Testican-1 | Q08629 | SPOCK1 |
| SL010388 | Trypsin 2 | Trypsin-2 | P07478 | PRSS2 |
| SL010390 | URB | Coiled-coil domain-containing protein | Q76M96 | CCDC80 |
| | | 80 | | |
| SL010391 | WFKN2 | WAP, Kazal, immunoglobulin, Kunitz | Q8TEU8 | WFIKKN2 |
| | | and NTR domain-containing protein 2 | | |
| SL010393 | KREM2 | Kremen protein 2 | Q8NCW0 | KREMEN2 |
| SL010449 | Carbonic | Carbonic anhydrase-related protein 10 | Q9NS85 | CA10 |
| | Anhydrase X | | | |
| SL010450 | CD48 | CD48 antigen | P09326 | CD48 |
| SL010451 | CFC1 | Cryptic protein | P0CG37 | CFC1 |
| SL010454 | Contactin-4 | Contactin-4 | Q8IWV2 | CNTN4 |
| SL010455 | Contactin-5 | Contactin-5 | O94779 | CNTN5 |
| SL010456 | CYTN | Cystatin-SN | P01037 | CST1 |
| SL010457 | DLL4 | Delta-like protein 4 | Q9NR61 | DLL4 |
| SL010458 | Endocan | Endothelial cell-specific molecule 1 | Q9NQ30 | ESM1 |
| SL010461 | FCGR1 | High affinity immunoglobulin gamma | P12314 | FCGR1A |
| | | Fc receptor | | |
| SL010462 | FCN1 | Ficolin-1 | O00602 | FCN1 |
| SL010463 | GPC2 | Glypican-2 | Q8N158 | GPC2 |
| SL010464 | LRIG3 | Leucine-rich repeats and | Q6UXM1 | LRIG3 |
| | | immunoglobulin-like | | |
| SL010465 | MATN2 | Matrilin-2 | O00339 | MATN2 |
| SL010466 | MFRP | Membrane frizzled-related protein | Q9BY79 | MFRP |
| SL010467 | RGMA | Repulsive guidance molecule A | Q96B86 | RGMA |
| SL010468 | RGMB | RGM domain family member B | Q6NW40 | RGMB |
| SL010469 | RGM-C | Hemojuvelin | Q6ZVN8 | HFE2 |
| SL010470 | Semaphorin 3E | Semaphorin-3E | O15041 | SEMA3E |
| SL010471 | Testican-2 | Testican-2 | Q92563 | SPOCK2 |
| SL010488 | ABL2 | Abelson tyrosine-protein kinase 2 | P42684 | ABL2 |
| SL010489 | CAMK1 | Calcium/calmodulin-dependent protein | Q14012 | CAMK1 |
| | | kinase type 1 | | |
| SL010490 | CAMK1D | Calcium/calmodulin-dependent protein | Q8IU85 | CAMKID |
| | | kinase type 1D | | |
| SL010491 | CAMK2A | Calcium/calmodulin-dependent protein | Q9UQM7 | CAMK2A |
| | | kinase type II subunit alpha | | |
| SL010492 | CAMK2B | Calcium/calmodulin-dependent protein | Q13554 | CAMK2B |
| | | kinase type II subunit beta | | |
| SL010493 | CAMK2D | Calcium/calmodulin-dependent protein | Q13557 | CAMK2D |
| | | kinase type II subunit delta | | |
| SL010494 | CDK1/cyclin B | Cyclin-dependent kinase 1:G2/mitotic- | P06493 | CDC2 CCNB1 |
| | | specific | P14635 | |
| SL010495 | CDK2/cyclin A | Cyclin-dependent kinase 2:Cyclin-A2 | P24941 | CDK2 CCNA2 |
| | | complex | P20248 | |
| SL010496 | CDK5/p35 | Cyclin-dependent kinase 5:Cyclin- | Q00535 | CDK5 CDK5R1 |
| | | dependent kinase 5 activator 1 complex | Q15078 | |
| SL010498 | EPHA3 | Ephrin type-A receptor 3 | P29320 | EPHA3 |
| SL010499 | HCK | Tyrosine-protein kinase HCK | P08631 | HCK |
| SL010500 | LYNB | Tyrosine-protein kinase Lyn, isoform B | P07948 | LYN |
| SL010501 | MP2K2 | Dual specificity mitogen-activated | P36507 | MAP2K2 |
| | | protein kinase kinase 2 | | |
| SL010502 | MK08 | Mitogen-activated protein kinase 8 | P45983 | MAPK8 |
| SL010503 | MAPK2 | MAP kinase-activated protein kinase 2 | P49137 | MAPKAPK2 |
| SL010504 | MAPK5 | MAP kinase-activated protein kinase 5 | Q8IW41 | MAPKAPK5 |
| SL010505 | MATK | Megakaryocyte-associated tyrosine- | P42679 | MATK |
| | | protein kinase | | |
| SL010508 | PAK3 | Serine/threonine-protein kinase PAK 3 | O75914 | PAK3 |
| SL010509 | PAK6 | Serine/threonine-protein kinase PAK 6 | Q9NQU5 | PAK6 |
| SL010510 | PAK7 | Serine/threonine-protein kinase PAK 7 | Q9P286 | PAK7 |
| SL010512 | PIK3CA/ | Phosphatidylinositol 4,5-bisphosphate 3- | P42336 | PIK3CA PIK3 |
| | PIK3R1 | kinase catalytic subunit alpha | P27986 | |
| | | isoform:Phosphatidylinositol 3-kinase | | |
| | | regulatory subunit alpha complex | | |
| SL010513 | PRKACA | cAMP-dependent protein kinase | P17612 | PRKACA |
| | | catalytic subunit A | | |
| SL010514 | PTK6 | Protein-tyrosine kinase 6 | Q13882 | PTK6 |
| SL010515 | RPS6KA3 | Ribosomal protein S6 kinase alpha-3 | P51812 | RPS6KA3 |
| SL010516 | SRCN1 | Proto-oncogene tyrosine-protein kinase | P12931 | SRC |
| | | Src | | |
| SL010517 | STK16 | Serine/threonine-protein kinase 16 | O75716 | STK16 |
| SL010518 | TEC | Tyrosine-protein kinase Tec | P42680 | TEC |
| SL010519 | ZAP70 | Tyrosine-protein kinase ZAP-70 | P43403 | ZAP70 |
| SL010520 | AURKB | Aurora kinase B | Q96GD4 | AURKB |
| SL010521 | BTK | Tyrosine-protein kinase BTK | Q06187 | BTK |
| SL010522 | CDK8/cyclin | Cyclin-dependent kinase 8:Cyclin-C | P49336 | CDK8 CCNC |
| | C | complex | P24863 | |
| SL010523 | HIPK3 | Homeodomain-interacting protein kinase | Q9H422 | HIPK3 |
| | | 3 | | |
| SL010528 | UFM1 | Ubiquitin-fold modifier 1 | P61960 | UFM1 |
| SL010529 | UFC1 | Ubiquitin-fold modifier-conjugating | Q9Y3C8 | UFC1 |
| | | enzyme 1 | | |
| SL010530 | OCAD1 | OCIA domain-containing protein 1 | Q9NX40 | OCIAD1 |
| SL010610 | CLC4K | C-type lectin domain family 4 member | Q9UJ71 | CD207 |
| | | K | | |
| SL010612 | Dkk-4 | Dickkopf-related protein 4 | Q9UBT3 | DKK4 |
| SL010613 | IL-17 RD | Interleukin-17 receptor D | Q8NFM7 | IL17RD |
| SL010616 | SHP-2 | Tyrosine-protein phosphatase non- | Q06124 | PTPN11 |
| | | receptor type 11 | | |
| SL010617 | TPSB2 | Tryptase beta-2 | P20231 | TPSB2 |
| SL010619 | TPSG1 | Tryptase gamma | Q9NRR2 | TPSG1 |
| SL010830 | 41 | Protein 4.1 | P11171 | EPB41 |
| SL010927 | IMB1 | Importin subunit beta-1 | Q14974 | KPNB1 |
| SL010928 | IMDH2 | Inosine-5′-monophosphate | P12268 | IMPDH2 |
| | | dehydrogenase 2 | | |
| SL010973 | PSA1 | Proteasome subunit alpha type-1 | P25786 | PSMA1 |
| SL011049 | MASP3 | Mannan-binding lectin serine protease 1 | P48740 | MASP1 |
| SL011068 | IL-17 RC | Interleukin-17 receptor C | Q8NAC3 | IL17RC |
| SL011069 | Marapsin | Serine protease 27 | Q9BQR3 | PRSS27 |
| SL011071 | PDGF-CC | Platelet-derived growth factor C | Q9NRA1 | PDGFC |
| SL011073 | XPNPEP1 | Xaa-Pro aminopeptidase 1 | Q9NQW7 | XPNPEP1 |
| SL011100 | CD226 | CD226 antigen | Q15762 | CD226 |
| SL011202 | SNAA | Alpha-soluble NSF attachment protein | P54920 | NAPA |
| SL011211 | IF4G2 | Eukaryotic translation initiation factor 4 | P78344 | EIF4G2 |
| | | gamma 2 | | |
| SL011232 | CDC37 | Hsp90 co-chaperone Cdc37 | Q16543 | CDC37 |
| SL011404 | PDE4D | CAMP-specific 3′,5′-cyclic | Q08499 | PDE4D |
| | | phosphodiesterase | | |
| SL011405 | PDE5A | cGMP-specific 3′,5′-cyclic | O76074 | PDE5A |
| | | phosphodiesterase | | |
| SL011406 | PDE7A | High affinity cAMP-specific 3′,5′-cyclic | Q13946 | PDE7A |
| | | phosphate | | |
| SL011448 | TNR4 | Tumor necrosis factor receptor | P43489 | TNFRSF4 |
| | | superfamily me | | |
| SL011498 | PACAP-38 | Pituitary adenylate cyclase-activating | P18509 | ADCYAP1 |
| | | polypeptide 38 | | |
| SL011499 | PH | Pancreatic hormone | P01298 | PPY |
| SL011508 | PACAP-27 | Pituitary adenylate cyclase-activating | P18509 | ADCYAP1 |
| | | polypeptide 27 | | |
| SL011509 | PYY | Peptide YY | P10082 | PYY |
| SL011510 | Somatostatin- | Somatostatin-28 | P61278 | SST |
| | 28 | | | |
| SL011528 | RS7 | 40S ribosomal protein S7 | P62081 | RPS7 |
| SL011529 | SBDS | Ribosome maturation protein SBDS | Q9Y3A5 | SBDS |
| SL011530 | DLRB1 | Dynein light chain roadblock-type 1 | Q9NP97 | DYNLRB1 |
| SL011532 | ETHE1 | Persulfide dioxygenase ETHE1, | O95571 | ETHE1 |
| | | mitochondrial | | |
| SL011533 | SGTA | Small glutamine-rich tetratricopeptide | O43765 | SGTA |
| | | repeat | | |
| SL011535 | RBM39 | RNA-binding protein 39 | Q14498 | RBM39 |
| SL011549 | ARI3A | AT-rich interactive domain-containing | Q99856 | ARID3A |
| | | protein | | |
| SL011616 | IF4A3 | Eukaryotic initiation factor 4A-III | P38919 | EIF4A3 |
| SL011628 | DBNL | Drebrin-like protein | Q9UJU6 | DBNL |
| SL011629 | AIP | AH receptor-interacting protein | O00170 | AIP |
| SL011630 | SE6L2 | Seizure 6-like protein 2 | Q6UXD5 | SEZ6L2 |
| SL011631 | NACA | Nascent polypeptide-associated complex | Q13765 | NACA |
| | | subunit | | |
| SL011708 | ARP19 | CAMP-regulated phosphoprotein 19 | P56211 | ARPP19 |
| SL011709 | PLPP | Pyridoxal phosphate phosphatase | Q96GD0 | PDXP |
| SL011768 | NUDC3 | NudC domain-containing protein 3 | Q8IVD9 | NUDCD3 |
| SL011769 | AN32B | Acidic leucine-rich nuclear | Q92688 | ANP32B |
| | | phosphoprotein 32 | | |
| SL011770 | LCMT1 | Leucine carboxyl methyltransferase 1 | Q9UIC8 | LCMT1 |
| SL011772 | PESC | Pescadillo homolog | O00541 | PES1 |
| SL011808 | CPNE1 | Copine-1 | Q99829 | CPNE1 |
| SL011809 | XTP3A | dCTP pyrophosphatase 1 | Q9H773 | DCTPP1 |
| SL012108 | PLCG1 | 1-phosphatidylinositol 4,5-bisphosphate | P19174 | PLCG1 |
| | | phosphate | | |
| SL012168 | LIN7B | Protein lin-7 homolog B | Q9HAP6 | LIN7B |
| SL012188 | EP15R | Epidermal growth factor receptor | Q9UBC2 | EPS15L1 |
| | | substrate 15 | | |
| SL012248 | FAK1 | Focal adhesion kinase 1 | Q05397 | PTK2 |
| SL012457 | NXPH1 | Neurexophilin-1 | P58417 | NXPH1 |
| SL012469 | GPC5 | Glypican-5 | P78333 | GPC5 |
| SL012538 | ARMEL | Cerebral dopamine neurotrophic factor | Q49AH0 | CDNF |
| SL012698 | KI2L4 | Killer cell immunoglobulin-like receptor | Q99706 | KIR2DL4 |
| | | 2DL4 | | |
| SL012707 | PCSK9 | Proprotein convertase subtilisin/kexin | Q8NBP7 | PCSK9 |
| | | type 9 | | |
| SL012740 | ATS15 | A disintegrin and metalloproteinase with | Q8TE58 | ADAMTS15 |
| | | thrombospondin motifs 15 | | |
| SL012754 | ASM3A | Acid sphingomyelinase-like | Q92484 | SMPDL3A |
| | | phosphodiesterase | | |
| SL012783 | WFKN1 | WAP, kazal, immunoglobulin, kunitz | Q96NZ8 | WFIKKN1 |
| | | and NTR domain-containing protein 1 | | |
| SL012822 | BSSP4 | Brain-specific serine protease 4 | Q9GZN4 | PRSS22 |
| SL013240 | CRK | Adapter molecule crk | P46108 | CRK |
| SL013488 | CLC1B | C-type lectin domain family 1 member B | Q9P126 | CLEC1B |
| SL013489 | AMNLS | Protein amnionless | Q9BXJ7 | AMN |
| SL013490 | BOC | Brother of CDO | Q9BWV1 | BOC |
| SL013548 | IL-34 | Interleukin-34 | Q6ZMJ4 | IL34 |
| SL013570 | DYRK3 | Dual specificity tyrosine- | 043781 | DYRK3 |
| | | phosphorylation-reg | | |
| SL013754 | RASA1 | Ras GTPase-activating protein 1 | P20936 | RASA1 |
| SL013928 | PPIE | Peptidyl-prolyl cis-trans isomerase E | Q9UNP9 | PPIE |
| SL013969 | KYNU | Kynureninase | Q16719 | KYNU |
| SL013988 | CHST2 | Carbohydrate sulfotransferase 2 | Q9Y4C5 | CHST2 |
| SL013989 | RSPO2 | R-spondin-2 | Q6UXX9 | RSPO2 |
| SL014008 | FUT5 | Alpha-(1,3)-fucosyltransferase 5 | Q11128 | FUT5 |
| SL014009 | HDGR2 | Hepatoma-derived growth factor-related | Q7Z4V5 | HDGFRP2 |
| | | protein | | |
| SL014028 | ENTP5 | Ectonucleoside triphosphate | O75356 | ENTPD5 |
| | | diphosphohydrolase | | |
| SL014029 | SPHK2 | Sphingosine kinase 2 | Q9NRA0 | SPHK2 |
| SL014048 | CONA1 | Collagen alpha-1(XXIII) chain | Q86Y22 | COL23A1 |
| SL014069 | PCSK7 | Proprotein convertase subtilisin/kexin | Q16549 | PCSK7 |
| | | type 7 | | |
| SL014070 | SLIK5 | SLIT and NTRK-like protein 5 | O94991 | SLITRK5 |
| SL014071 | FLRT1 | Leucine-rich repeat transmembrane | Q9NZU1 | FLRT1 |
| | | protein FLR | | |
| SL014088 | FCRL3 | Fc receptor-like protein 3 | Q96P31 | FCRL3 |
| SL014091 | SORC2 | VPS10 domain-containing receptor | Q96PQ0 | SORCS2 |
| | | SorCS2 | | |
| SL014092 | CDON | Cell adhesion molecule-related/down- | Q4KMG0 | CDON |
| | | regulated | | |
| SL014093 | ENTP3 | Ectonucleoside triphosphate | O75355 | ENTPD3 |
| | | diphosphohydrolase | | |
| SL014094 | GP114 | Probable G-protein coupled receptor 114 | Q8IZF4 | GPR114 |
| SL014096 | LRRT1 | Leucine-rich repeat transmembrane | Q86UE6 | LRRTM1 |
| | | neuronal protein 1 | | |
| SL014108 | LRRT3 | Leucine-rich repeat transmembrane | Q86VH5 | LRRTM3 |
| | | neuronal protein 3 | | |
| SL014111 | KIRR3 | Kin of IRRE-like protein 3 | Q8IZU9 | KIRREL3 |
| SL014113 | NLGNX | Neuroligin-4, X-linked | Q8N0W4 | NLGN4X |
| SL014129 | H6ST1 | Heparan-sulfate 6-O-sulfotransferase 1 | O60243 | HS6ST1 |
| SL014130 | CHST6 | Carbohydrate sulfotransferase 6 | Q9GZX3 | CHST6 |
| SL014148 | ROBO3 | Roundabout homolog 3 | Q96MS0 | ROBO3 |
| SL014208 | CRTAM | Cytotoxic and regulatory T-cell | 095727 | CRTAM |
| | | molecule | | |
| SL014209 | KLRF1 | Killer cell lectin-like receptor subfamily | Q9NZS2 | KLRF1 |
| | | F | | |
| SL014228 | SLAF6 | SLAM family member 6 | Q96DU3 | SLAMF6 |
| SL014268 | OX2G | OX-2 membrane glycoprotein | P41217 | CD200 |
| SL014269 | KI3L2 | Killer cell immunoglobulin-like receptor | P43630 | KIR3DL2 |
| | | 3DL2 | | |
| SL014270 | CLM6 | CMRF35-like molecule 6 | Q08708 | CD300C |
| SL014288 | MO2R1 | Cell surface glycoprotein CD200 | Q8TD46 | CD200R1 |
| | | receptor 1 | | |
| SL014289 | KI3S1 | Killer cell immunoglobulin-like receptor | Q14943 | KIR3DS1 |
| | | 3DS1 | | |
| SL014292 | SIG14 | Sialic acid-binding Ig-like lectin 14 | Q08ET2 | SIGLEC14 |
| SL014294 | EPHAA | Ephrin type-A receptor 10 | Q5JZY3 | EPHA10 |
| SL014308 | FGF-8A | Fibroblast growth factor 8 isoform A | P55075 | FGF8 |
| SL014468 | SH21A | SH2 domain-containing protein 1A | O60880 | SH2D1A |
| SL014469 | SHC1 | SHC-transforming protein 1 | P29353 | SHC1 |
| SL014470 | BCAR3 | Breast cancer anti-estrogen resistance | O75815 | BCAR3 |
| | | protein | | |
| SL014735 | IMDH1 | Inosine-5′-monophosphate | P20839 | IMPDH1 |
| | | dehydrogenase 1 | | |
| SL015728 | GCKR | Glucokinase regulatory protein | Q14397 | GCKR |
| SL016128 | TXD12 | Thioredoxin domain-containing protein | O95881 | TXNDC12 |
| | | 12 | | |
| SL016129 | FAM107B | Protein FAM107B | Q9H098 | FAM107B |
| SL016130 | BRF-1 | Transcription factor IIIB 90 kDa subunit | Q92994 | BRF1 |
| SL016148 | C34 gp41 | gp41 C34 peptide, HIV | Q70626 | Human-virus |
| | HIV | | | |
| | Fragment | | | |
| SL016548 | AMPK | AMP Kinase (alpha1beta1gamma1) | Q13131 | PRKAA1 PRKA |
| | alb1g1 | | Q9Y478 | |
| | | | P54619 | |
| SL016549 | AMPK | AMP Kinase (alpha2beta2gamma1) | P54646 | PRKAA2 PRKA |
| | a2b2g1 | | O43741 | |
| | | | P54619 | |
| SL016550 | CK2-A1:B | Casein kinase II 2-alpha:2-beta | P68400 | CSNK2A1 CSN |
| | | heterotetramer | P67870 | |
| SL016551 | CK2-A2:B | Casein kinase II 2-alpha′:2-beta | P19784 | CSNK2A2 CSN |
| | | heterotetramer | P67870 | |
| SL016553 | PDE3A | cGMP-inhibited 3′,5′-cyclic | Q14432 | PDE3A |
| | | phosphodiesterase | | |
| SL016554 | PDE9A | High affinity cGMP-specific 3′,5′-cyclic | O76083 | PDE9A |
| | | phosphodiesterase | | |
| SL016555 | PDE11 | Dual 3′,5′-cyclic-AMP and -GMP | Q9HCR9 | PDE11A |
| | | phosphodiester | | |
| SL016557 | HMGR | 3-hydroxy-3-methylglutaryl-coenzyme | P04035 | HMGCR |
| | | A reductase | | |
| SL016563 | GHC2 | Mitochondrial glutamate carrier 2 | Q9H1K4 | SLC25A18 |
| SL016566 | DRAK2 | Serine/threonine-protein kinase 17B | 094768 | STK17B |
| SL016567 | TAK1- | Mitogen-activated protein kinase kinase | 043318 | MAP3K7 TAB1 |
| | TAB1 | kinase | Q15750 | |
| SL016928 | SLAF7 | SLAM family member 7 | Q9NQ25 | SLAMF7 |
| SL017188 | GSK-3 | Glycogen synthase kinase-3 alpha/beta | P49840 | GSK3A GSK3B |
| | alpha/beta | | P49841 | |
| SL017189 | Kininogen, | Kininogen-1 | P01042 | KNG1 |
| | HMW | | | |
| SL017610 | Gro-b/g | Gro-beta/gamma | P19876 | CXCL3 CXCL2 |
| | | | P19875 | |
| SL017611 | 14-3-3 | 14-3-3 protein family | P31946, | YWHAB |
| | | | P62258, | YWHAE |
| | | | P61981 | |
| SL017612 | HSP 90a/b | Heat shock protein HSP 90-alpha/beta | P07900 | HSP90AA1 HS |
| | | | P08238 | |
| SL017613 | FCG2A/B | Low affinity immunoglobulin gamma Fc | P12318 | FCGR2A FCGR |
| | | region r | P31994 | |
| SL017614 | PKB a/b/g | Protein kinase B alpha/beta/gamma | | |
| SL018548 | alpha-1- | alpha-1-antichymotrypsin complex | P07288, | SERPINA3 |
| | antichymotryp | | P01011 | |
| SL018625 | TLR4:MD- | Toll-like receptor 4:Lymphocyte antigen | O00206 | TLR4 LY96 |
| | 2 complex | 96 co | Q9Y6Y9 | |

### Statistical Analysis

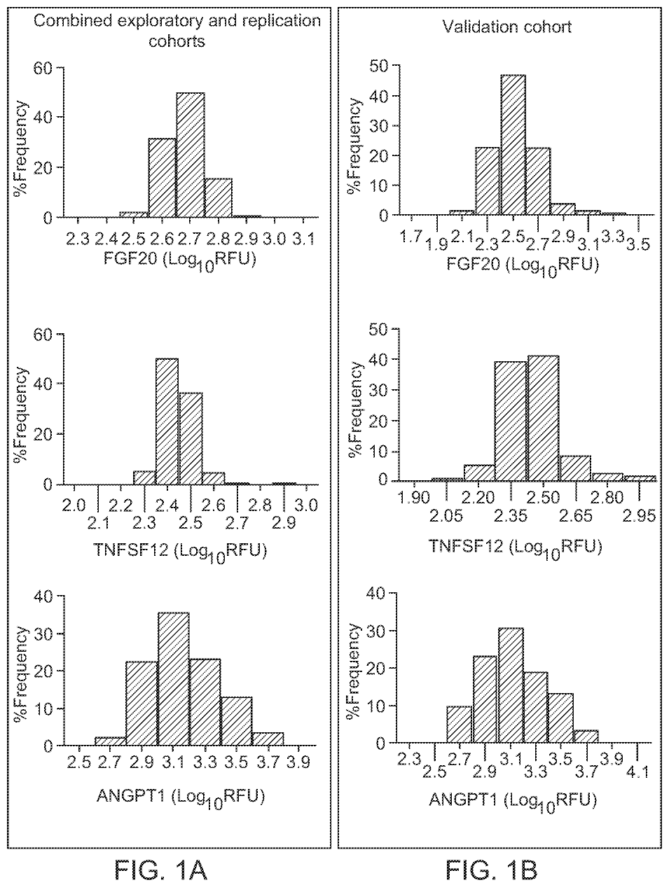

All statistical analyses were performed using SAS for Windows, version 9.4 (SAS Institute, Cary, NC). All data were presented as either mean and standard deviations, median (25^{th} and 75^{th} percentiles) or count (proportion) measures, where applicable. Correlations between circulating plasma concentrations with eGFR slopes, TNF-R1 and clinical covariates were assessed using a Spearman's rank correlation (rₛ). Clusters of protective proteins were identified using a hierarchical cluster analysis (Ward's method). Baseline protein RFU concentrations (n=1,129) were natural log transformed and then were categorized into quartiles of their distributions prior to association testing. The distributions of the top 3 protective proteins after natural log transformation in the combined discovery and replication cohorts, and in the validation cohort are shown in FIGS. 1A-1B. Univariate and multivariable logistic regression models were used to test associations of relevant circulating plasma proteins measured at baseline with the outcome measure (being a progressor, if eGFR loss ≥3.0 ml/min/year or progression to ESKD), and expressed as odds ratios per one quartile increase in circulating plasma concentration of the relevant protein with corresponding 95% confidence intervals. The cumulative incidence rate of ESKD according to the index of protection—the combined effect of the three exemplar protective proteins, was analyzed using PROC LIFETEST in SAS software. Comparisons between plasma protein concentrations in non-diabetics, non-progressors and progressors were examined using one-way ANOVA with Dunn's multiple comparisons test. Significance was defined as *P<0.05, **P<0.01, ***P<0.001, and ****P<0.0001.

### Example 1. Characteristics of the Exploratory and Replication Cohorts of Joslin Kidney Study

The study disclosed herein included subjects participating in the ongoing Joslin Kidney Study. Two independent cohorts of subjects with diabetes and impaired kidney function (CKD Stage 3) were assembled; an exploratory Joslin cohort of 214 subjects with T1D and a replication Joslin cohort of 144 subjects with T2D. These cohorts were followed for 7-15 years to determine eGFR slope and ascertain time of onset of ESKD. The clinical characteristics of these cohorts are shown in Table 2. All study participants included in the Joslin T1D cohort and 92% of study participants in the T2D cohort were Caucasian. At baseline, in comparison with subjects with T1D, those with T2D were older, had shorter duration of diabetes, higher body mass index (BMI), lower hemoglobin A1c (HbA1c) and lower urinary albumin to creatinine ratio (ACR) but similarly impaired eGFR.

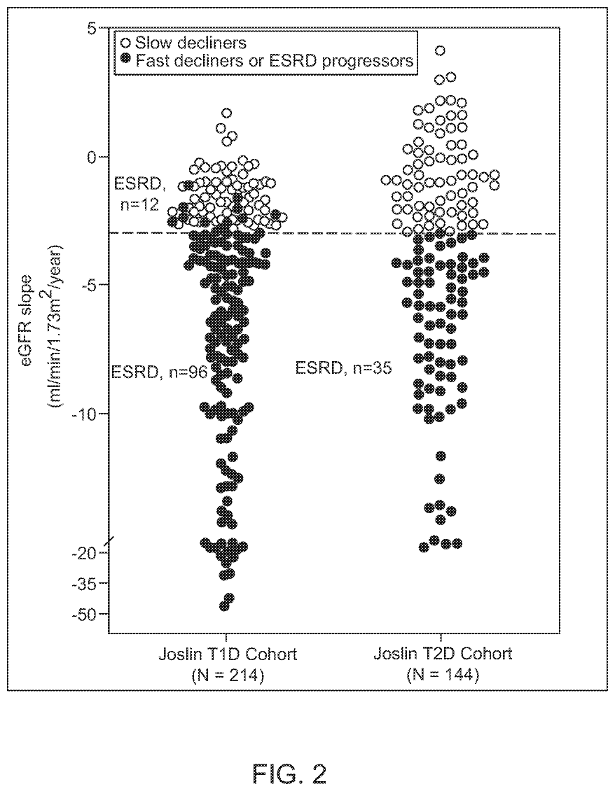

During 7-15 years of follow-up, majority of subjects in both cohorts had progressive renal decline. However, eGFR slopes varied greatly among subjects, with slopes being slightly steeper in subjects with T1D than in those with T2D. The distribution of eGFR slopes in the Joslin cohorts with T1D and T2D is described in FIG. 2. The number of slow decliners (referred to as non-progressors) defined as eGFR loss <3.0 ml/min/year was 71 (33%) and 69 (48%) in the T1D exploratory and T2D replication cohorts, respectively (Table 2). These non-progressors, the focus of this research, had very shallow eGFR slopes, with the median (25^{th}, 75^{th} percentile) being −1.6 ml/min/year (−2.3, −1.0) and −0.9 ml/min/year (−2.0, 0.4) in T1D and T2D cohorts, respectively. None of these subjects progressed to ESKD during the 7-15 years of follow-up. In contrast, a large proportion (61% of combined cohorts) of fast decliners (referred to as progressors) defined as eGFR loss ≥3.0 ml/min/year progressed to ESKD within 10 years of follow-up, as described in Table 2.

TABLE 2
Demographics and clinical characteristics of the
Joslin Kidney Study cohorts with T1D and T2D.

| | EXPLORATORY | | REPLICATION | | |
|---|---|---|---|---|---|
| | Joslin T1D Cohort | | Joslin T2D Cohort | | |
| Characteristics | (N = 214) | | (N = 144) | | P-value |
| At baseline | | | | | |
| Male, n (%) | 104 | (49%) | 94 | (65%) | 0.002 |
| Ethnicity | | | | | <0.0001 |
| Caucasian, n (%) | 214 | (100%) | 132 | (92%) | |
| Non-Caucasian, n (%) | 0 | (0%) | 12 | (8%) | |
| Age at DM onset (years) | 13 | (8, 20) | 44 | (38, 50) | <0.0001 |
| Age at study entry (years) | 44 | (38, 51) | 61 | (56, 64) | <0.0001 |
| Duration of diabetes (years) | 28 | (23, 36) | 15 | (11, 21) | <0.0001 |
| BMI (kg/m²) | 26.4 | (23, 28) | 33.4 | (29, 37) | <0.0001 |
| Systolic BP (mm Hg) | 133 | (124, 147) | 139 | (128, 150) | 0.02 |
| Diastolic BP (mm Hg) | 78 | (70, 84) | 74 | (69, 81) | 0.04 |
| Insulin Rx, % | 100% | | 65% | | <0.0001 |
| Renoprotection Rx, % | 81% | | 86% | | 0.19 |
| HbA1c (%) | 8.6 | (7.7, 9.6) | 7.3 | (6.7, 8.3) | <0.0001 |
| ACR (mg/g creatinine) | 795 | (274, 1803) | 255 | (57, 1096) | <0.0001 |
| eGFR (ml/min/1.73 m²) | 43.2 | (35, 51) | 48.7 | (40, 57) | <0.0001 |
| During follow-up | | | | | |
| eGFR slope (ml/min/1.73 m²/year) | −4.0 | (−7.8, −2.1) | −3.1 | (−6.4, −0.9) | 0.007 |
| Non-progressors^{a}, n (%) | 71 | (33%) | 69 | (48%) | |
| Progressors^{a}, n (%) | 143 | (67%) | 75 | (52%) | |
| New incidence of ESKD during 10- | 108 | (50%) | 35 | (24%) | <0.0001 |
| year follow-up, n (%) | | | | | |
| Deaths unrelated to ESKD, n (%) | 15 | (7%) | 8 | (6%) | 0.58 |

T1D, Type 1 diabetes; T2D, Type 2 diabetes; DM, Diabetes mellitus; BMI, Body mass index; BP, Blood pressure; Rx, treatment; Renoprotection, Prescription of angiotensin-converting enzyme inhibitor (ACE-I) or angiotensin II receptor blocker (ARB); HbA1c, Hemoglobin A1c; ACR, Albumin-to-creatinine ratio; eGFR, Estimated glomerular filtration rate; ESKD, End-stage kidney disease.
^{a}Non-progressors were defined as eGFR loss <3.0 ml/min/1.73 m²/year and Progressors as eGFR loss ≥3.0 ml/min/1.73 m²/year. Data presented as median (25^{th}, 75^{th} percentile) or count (proportion) measures.

Differences between the two cohorts were tested using the Wilcoxon-rank-sum test for continuous variables, and the χ² test for categorical variables.

### Example 2. Profiling Plasma Proteins that Protect Against Progressive Renal Decline

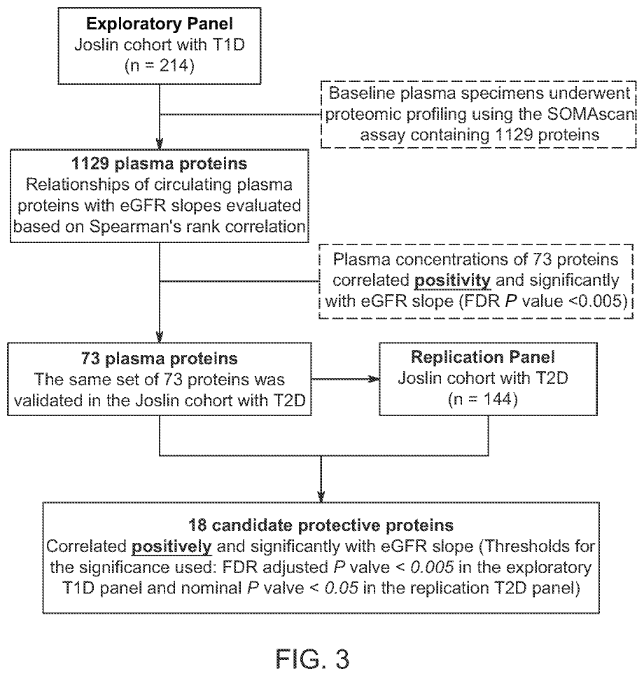

The SOMAscan proteomic platform was used to measure 1129 plasma proteins, as described in Table 1 above. These plasma proteins were examined for elevated concentration in non-progressors at baseline. The schematic representation of this study is outlined in FIG. 3. In the Joslin exploratory T1D cohort, baseline plasma concentration of 73 proteins were positively and significantly correlated with eGFR slope at a false discovery rate (FDR) adjusted P<0.005 (Table 3), therefore, elevated baseline concentrations of these proteins were associated with slow or minimal renal decline during follow-up. These proteins can be considered candidate protective factors/biomarkers against progressive renal decline. Proteins that were negatively correlated with eGFR slope might be considered candidate factors/biomarkers increasing the risk of progressive renal decline and progression to ESKD. Rather, a separate study has published the association of 194 inflammatory circulating proteins with the risk of progression to ESKD in these two Joslin cohorts using the same SOMAscan proteomic platform (Niewczas et al., Nat Med 25: 805-813 (2019)).

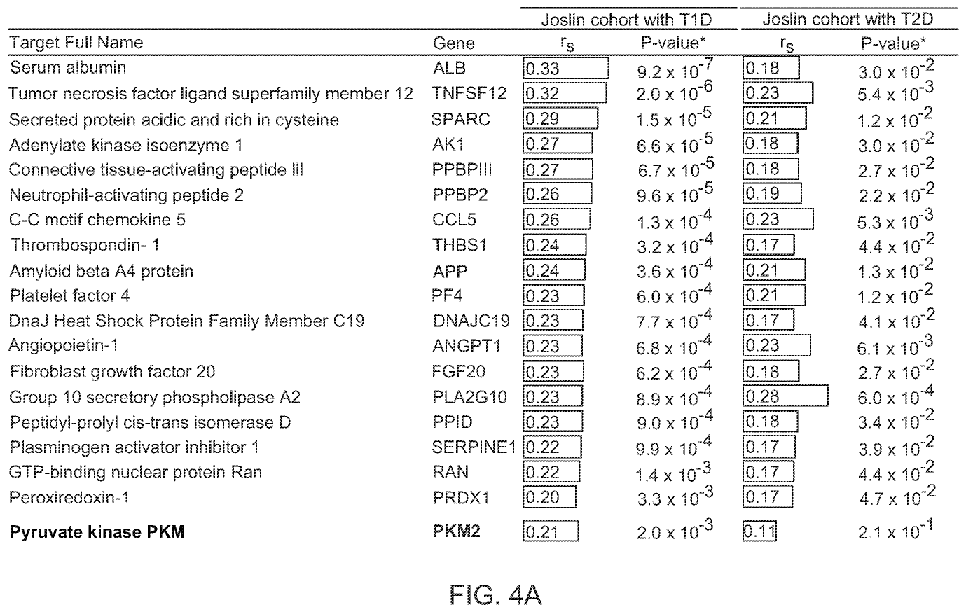

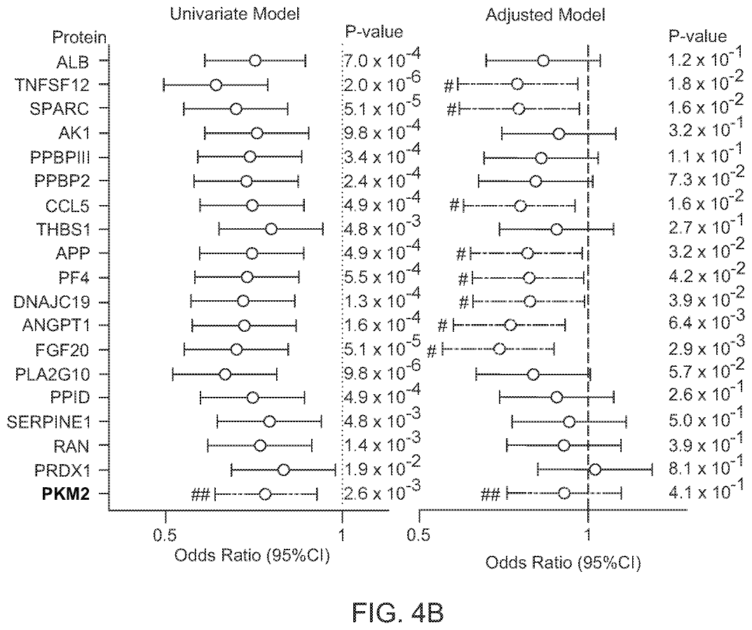

The 73 plasma proteins positively correlated with eGFR slope in subjects with T1D were analyzed further in the replication cohort of subjects with T2D. Eighteen proteins were found positively correlated with eGFR slope at a nominal P<0.05 (Table 3). As discussed herein, elevated concentrations of PKM2 in kidney tissue and in plasma were recently demonstrated as a novel biomarker and potential therapeutic target protecting against DKD in subjects with long duration of T1D (Qi et al., Nat Med 23: 753-762 (2017)). To determine whether this protein may be also involved in protection against progressive renal decline in subjects with impaired kidney function, PKM2, along with the 18 candidate proteins were included, in further analyses despite its non-significant correlation with eGFR slope in subjects with T2D. The names of 19 plasma proteins, correlation coefficients and P-values for each positively correlated protein with eGFR slope in the T1D and T2D cohorts, respectively, are presented in FIG. 4A. Correlations were generally slightly weaker in those with T2D, but all 18 proteins correlated positively and significantly with eGFR slope.

TABLE 3
Global proteomic profiling data of the circulating plasma proteins
in the exploratory cohort of 214 T1D subjects and in the replication
cohort of 144 T2D subjects. Spearman's rank correlation coefficients
(rₛ) between baseline concentration of 73 proteins and eGFR slope.

| | | Joslin T1D Cohort | | Joslin T2D Cohort | |
|---|---|---|---|---|---|
| UniProt ID | Gene Symbol | rₛ | P-value* | rₛ | P-value* |
| P02768 | ALB | 0.33 | 9.20E−07 | 0.18 | 3.01E−02 |
| O43508 | TNFSF12 | 0.32 | 2.00E−06 | 0.23 | 5.40E−03 |
| P09486 | SPARC | 0.29 | 1.50E−05 | 0.21 | 1.15E−02 |
| P00568 | AK1 | 0.27 | 6.60E−05 | 0.18 | 3.04E−02 |
| P02775 | PPBPIII | 0.27 | 6.70E−05 | 0.18 | 2.65E−02 |
| P02775 | PPBP2 | 0.26 | 9.60E−05 | 0.19 | 2.18E−02 |
| P13501 | CCL5 | 0.26 | 1.30E−04 | 0.23 | 5.30E−03 |
| P07996 | THBS1 | 0.24 | 3.20E−04 | 0.17 | 4.35E−02 |
| P05067 | APP | 0.24 | 3.60E−04 | 0.21 | 1.34E−02 |
| P02776 | PF4 | 0.23 | 6.00E−04 | 0.21 | 1.17E−02 |
| Q9NP95 | FGF20 | 0.23 | 6.20E−04 | 0.18 | 2.71E−02 |
| Q15389 | ANGPT1 | 0.23 | 6.80E−04 | 0.23 | 6.10E−03 |
| Q96DA6 | DNAJC19 | 0.23 | 7.70E−04 | 0.17 | 4.09E−02 |
| O15496 | PLA2G10 | 0.23 | 8.90E−04 | 0.28 | 6.00E−04 |
| Q08752 | PPID | 0.23 | 9.00E−04 | 0.18 | 3.41E−02 |
| P05121 | SERPINE1 | 0.22 | 9.90E−04 | 0.17 | 3.88E−02 |
| P62826 | RAN | 0.22 | 1.40E−03 | 0.17 | 4.44E−02 |
| Q06830 | PRDX1 | 0.20 | 3.30E−03 | 0.17 | 4.72E−02 |
| P14618 | PKM2 | 0.21 | 2.00E−03 | 0.11 | 2.09E−01 |
| P24298 | GPT | 0.31 | 5.10E−06 | 0.07 | 3.91E−01 |
| P35625 | TIMP3 | 0.30 | 7.10E−06 | 0.10 | 2.27E−01 |
| P01857 | IGHG1 IGHG2 | 0.30 | 9.10E−06 | 0.16 | 5.45E−02 |
| P52209 | PGD | 0.28 | 4.20E−05 | 0.15 | 7.19E−02 |
| P19876 P19875 | CXCL3 CXCL2 | 0.27 | 8.10E−05 | 0.15 | 7.65E−02 |
| Q9UHL4 | DPP7 | 0.25 | 2.10E−04 | 0.09 | 2.67E−01 |
| P14210 | HGF | 0.25 | 2.30E−04 | 0.03 | 7.38E−01 |
| Q96RJ3 | TNFRSF13C | 0.25 | 2.70E−04 | 0.07 | 4.26E−01 |
| P62979 | RPS27A | 0.24 | 3.00E−04 | 0.02 | 8.05E−01 |
| P40925 | MDH1 | 0.24 | 3.30E−04 | 0.02 | 8.47E−01 |
| P49137 | MAPKAPK2 | 0.24 | 4.90E−04 | 0.11 | 2.07E−01 |
| Q9UJU6 | DBNL | 0.24 | 5.20E−04 | 0.04 | 6.24E−01 |
| P07355 | ANXA2 | 0.23 | 5.70E−04 | −0.05 | 5.84E−01 |
| P07384 P04632 | CAPN1 CAPNS | 0.23 | 5.90E−04 | 0.11 | 1.95E−01 |
| P30041 | PRDX6 | 0.23 | 6.20E−04 | 0.09 | 2.71E−01 |
| P29401 | TKT | 0.23 | 6.40E−04 | 0.02 | 8.10E−01 |
| Q9Y3A5 | SBDS | 0.23 | 6.60E−04 | 0.14 | 1.04E−01 |
| P51452 | DUSP3 | 0.23 | 7.00E−04 | 0.08 | 3.68E−01 |
| P69905, P68871 | HBA1 HBB | 0.23 | 8.50E−04 | 0.06 | 4.43E−01 |
| P61088 | UBE2N | 0.22 | 9.20E−04 | 0.03 | 7.13E−01 |
| P14550 | AKR1A1 | 0.22 | 9.30E−04 | −0.05 | 5.22E−01 |
| P09960 | LTA4H | 0.22 | 9.40E−04 | −0.32 | <.0001 |
| O60383 | GDF9 | 0.22 | 9.50E−04 | 0.02 | 8.28E−01 |
| O14929 | HAT1 | 0.22 | 9.80E−04 | 0.10 | 2.35E−01 |
| O15264 | MAPK13 | 0.22 | 1.10E−03 | −0.05 | 5.20E−01 |
| P50395 | GDI2 | 0.22 | 1.10E−03 | 0.02 | 8.10E−01 |
| P12931 | SRC | 0.22 | 1.20E−03 | 0.08 | 3.27E−01 |
| Q13421 | MSLN | 0.22 | 1.20E−03 | 0.00 | 1.00E+00 |
| P04040 | CAT | 0.22 | 1.30E−03 | −0.01 | 9.52E−01 |
| P60174 | TPI1 | 0.22 | 1.30E−03 | 0.01 | 9.08E−01 |
| Q93038 | TNFRSF25 | 0.22 | 1.30E−03 | 0.11 | 1.71E−01 |
| P22392 | NME2 | 0.22 | 1.40E−03 | 0.06 | 4.88E−01 |
| P02794 P02792 | FTH1 FTL | 0.22 | 1.40E−03 | 0.11 | 2.06E−01 |
| Q06323 | PSME1 | 0.22 | 1.50E−03 | 0.00 | 9.78E−01 |
| P62937 | PPIA | 0.21 | 1.60E−03 | 0.09 | 2.63E−01 |
| P78556 | CCL20 | 0.21 | 1.70E−03 | 0.05 | 5.46E−01 |
| P19784 P67870 | CSNK2A2 CSN | 0.21 | 1.70E−03 | 0.19 | 1.17E−01 |
| Q02083 | NAAA | 0.21 | 1.70E−03 | 0.07 | 3.92E−01 |
| Q15181 | PPA1 | 0.21 | 1.80E−03 | 0.04 | 6.29E−01 |
| Q16548 | BCL2A1 | 0.21 | 1.90E−03 | 0.03 | 7.24E−01 |
| P31948 | STIP1 | 0.21 | 2.10E−03 | 0.09 | 2.77E−01 |
| P63241 | EIF5A | 0.21 | 2.20E−03 | 0.06 | 4.82E−01 |
| P0C0S5 | H2AFZ | 0.21 | 2.20E−03 | −0.10 | 2.37E−01 |
| P56211 | ARPP19 | 0.21 | 2.50E−03 | 0.07 | 4.25E−01 |
| P17612 | PRKACA | 0.21 | 2.50E−03 | 0.03 | 6.81E−01 |
| P30086 | PEBP1 | 0.21 | 2.60E−03 | −0.02 | 7.77E−01 |
| P23528 | CFL1 | 0.21 | 2.60E−03 | −0.06 | 4.78E−01 |
| P54920 | NAPA | 0.21 | 2.60E−03 | 0.14 | 8.46E−02 |
| Q8N5S9 | CAMKK1 | 0.20 | 2.70E−03 | 0.11 | 1.81E−01 |
| P63000 | RAC1 | 0.20 | 2.70E−03 | 0.15 | 8.25E−02 |
| P62979 | RPS27A | 0.20 | 2.90E−03 | −0.02 | 8.13E−01 |
| P55008 | AIF1 | 0.20 | 2.90E−03 | −0.07 | 3.91E−01 |
| Q9UQ80 | PA2G4 | 0.20 | 3.00E−03 | 0.07 | 3.73E−01 |
| P14735 | IDE | 0.20 | 3.00E−03 | 0.06 | 4.78E−01 |

*Threshold for the significance used in cohort with T1D: FDR adjusted P-value <0.005 in the exploratory T1D cohort and a nominal P-value <0.05 in the replication T2D cohort. Coefficients (rₛ) are presented below and corresponding two-sided P values have been provided. Gene symbols indicated in bold were examined in the present study.

### Example 3. Plasma Proteins Protecting Against Progressive Renal Decline

As both Joslin cohorts had impaired kidney function (CKD Stage 3) at baseline and had homogenous strength of association with eGFR slope, the SOMAscan results from both cohorts were combined. The association of baseline plasma concentration of each of the 19 proteins and the rate of progressive renal decline was analyzed using the logistic regression analysis. Subjects from combined Joslin cohorts were grouped to those with (1) fast renal decline (eGFR loss ≥3.0 ml/min/year) or progression to ESKD, referred to as progressors; or (2) subjects with slow or minimal renal decline (eGFR loss <3.0 ml/min/year), referred to as non-progressors. To assess statistical independence of protective effect from clinical characteristics and risk factors associated with progressive renal decline, first univariate and then multivariable logistic models adjusted for baseline clinical covariates were performed. The list of potential confounders included age, gender, ethnicity/race, duration of diabetes, insulin treatment, renoprotection treatment, BMI, systolic and diastolic blood pressures, HbA1c, eGFR and ACR. The key covariates, consisting of HbA1c, eGFR and ACR were included in the final logistic model. Information about selection of covariates into the logistic models are provided in Table 4. The results of univariable and multivariable analyses are shown in FIG. 4B. All models were adjusted for type of diabetes. The effects are shown as odds ratios (OR) with 95% confidence interval (95% CI) per one quartile increase in baseline plasma concentration of the specific protein. In the univariate model, all 19 proteins including PKM2 (FIG. 4B-marked with ##) protected (had OR<1.0) against progressive renal decline. Elevated plasma concentrations of 8 proteins remained associated with protection against progressive renal decline in the final model adjusted for baseline clinical covariates including eGFR, HbA1c, ACR and type of diabetes (FIG. 4B and Table 5). These 8 plasma proteins, referred to as “confirmed” protective proteins, included TNFSF12, SPARC, CCL5, APP, PF4, DNAJC19, ANGPT1 and FGF20 (FIG. 4B-marked with #). Baseline concentrations of PKM2 were not associated with protection against progressive renal decline after further adjustment by clinical covariates. Although significant (P<0.05) in the univariate analysis, the effect of PKM2 became statistically non-significant after adjustment for clinical covariates.

TABLE 4
Selection of potential covariates into the logistic regression model.

| | Maximum Likelihood Estimates | | | |
|---|---|---|---|---|
| Potential covariates | Estimate | Standard Error | Wald Chi-square | P-value |
| Age | −0.02 | 0.02 | 0.94 | 0.33 |
| Gender | 0.04 | 0.27 | 0.02 | 0.90 |
| Ethnicity | 0.96 | 0.74 | 1.68 | 0.20 |
| Insulin Rx | −0.27 | 0.45 | 0.36 | 0.55 |
| Renoprotection Rx | 0.18 | 0.35 | 0.26 | 0.61 |
| Duration of diabetes | −0.02 | 0.02 | 0.67 | 0.41 |
| BMI | −0.01 | 0.02 | 0.33 | 0.56 |
| Systolic BP | −0.001 | 0.01 | 0.01 | 0.93 |
| Diastolic BP | 0.01 | 0.02 | 0.16 | 0.69 |
| HbA1c | 0.24 | 0.09 | 6.85 | 0.0089 |
| ACR | 1.10 | 0.19 | 33.96 | <.0001 |
| eGFR | −0.05 | 0.01 | 15.11 | 0.0001 |

BMI, Body mass index; BP, Blood pressure; Rx, treatment; Renoprotection, Prescription of angiotensin-converting enzyme inhibitor (ACE-I) or angiotensin II receptor blocker (ARB); HbA1c, Hemoglobin A1c; ACR, Albumin-to-creatinine ratio; eGFR, Estimated glomerular filtration rate.

The criteria to retain a covariate in the final model were statistical significance at nominal P<0.05 and by inspection of β estimates, such that a change of β of 20% or higher was considered non-negligible.

TABLE 5
Logistic regression models examining the association of
19 circulating plasma proteins and progressive renal decline
in the combined Joslin cohorts with T1D and T2D.

| | Model 1 | Model 2 |
|---|---|---|
| Gene symbol for proteins | OR (95% CI) | OR (95% CI) |
| ALB | 0.71 (0.58, 0.87) | 0.83 (0.66, 1.05) |
| TNFSF12 | 0.61 (0.50, 0.75) | 0.75 (0.59, 0.95)* |
| SPARC | 0.66 (0.54, 0.81) | 0.75 (0.59, 0.95)* |
| AK1 | 0.72 (0.59, 0.87) | 0.89 (0.70, 1.02) |
| PPBPIII | 0.70 (0.57, 0.85) | 0.83 (0.65, 1.04) |
| PPBP2 | 0.69 (0.56, 0.84) | 0.81 (0.64, 1.02) |
| CCL5 | 0.70 (0.58, 0.86) | 0.75 (0.60, 0.95)* |
| THBS1 | 0.75 (0.62, 0.92) | 0.88 (0.70, 1.11) |
| APP | 0.70 (0.58, 0.86) | 0.78 (0.61, 0.98)* |
| PF4 | 0.69 (0.56, 0.84) | 0.78 (0.62, 0.99)* |
| FGF20 | 0.66 (0.54, 0.81) | 0.69 (0.54, 0.88)* |
| ANGPT1 | 0.68 (0.56, 0.83) | 0.72 (0.57, 0.91)* |
| DNAJC19 | 0.68 (0.55, 0.83) | 0.78 (0.62, 0.99)* |
| PLA2G10 | 0.63 (0.52, 0.78) | 0.80 (0.63, 1.01) |
| PPID | 0.70 (0.58, 0.86) | 0.88 (0.70, 1.10) |
| SERPINE1 | 0.75 (0.62, 0.92) | 0.92 (0.73, 1.17) |
| RAN | 0.73 (0.60, 0.88) | 0.90 (0.71, 1.15) |
| PRDX1 | 0.79 (0.65, 0.96) | 1.03 (0.82, 1.30) |
| PKM2 | 0.74 (0.61, 0.90) | 0.91 (0.72, 1.15) |

The effect is shown as an odds ratio (95% CI) per one quartile increase in circulating concentration of the relevant protein. Model 1: Unadjusted; Model 2: Adjusted for baseline eGFR, HbA1c and ACR. All models were adjusted by type of diabetes. *Proteins in bold are significant (P<0.05) in both models.

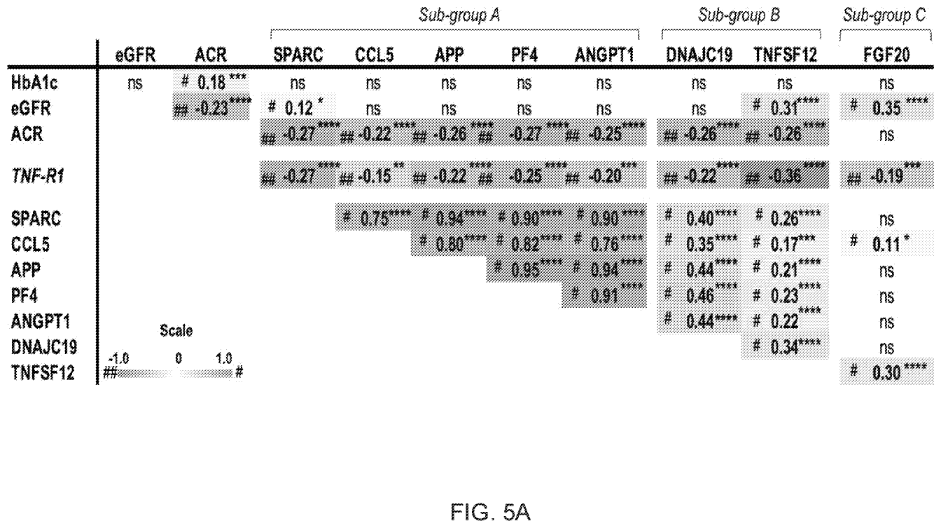

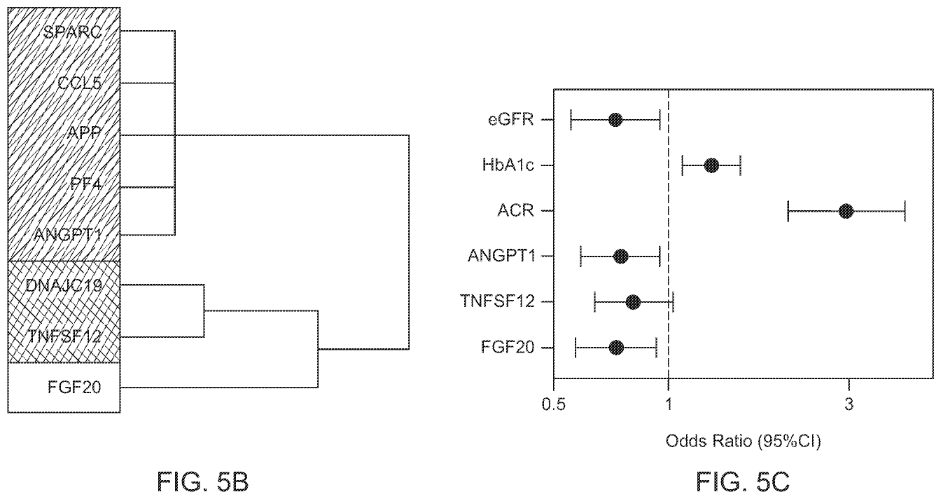

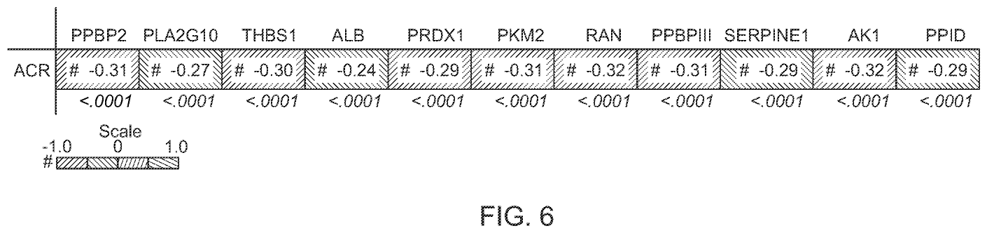

To examine which of the confirmed protective proteins contributed independently to protection against progressive renal decline, first, relationships at baseline were analyzed among the 8 proteins and important clinical covariates using a Spearman's rank correlation. The correlation matrix shown in FIG. 5A indicates that variation in baseline HbA1c had no impact on variation of the 8 protective proteins, whereas variation in baseline eGFR correlated weakly with TNFSF12 and FGF20. In contrast, baseline ACR correlated weakly with all of the proteins (FIG. 5A and FIG. 6) except for FGF20. In addition, all of the protective proteins correlated negatively with plasma tumor necrosis factor receptor 1 (TNF-R1) concentration, reported by us previously as one of the circulating inflammatory proteins associated with increased risk of progression to ESKD (Niewczas et al., Nat Med 25: 805-813 (2019)), indicating a decreased plasma TNF-R1 concentrations with increasing concentrations of the protective proteins. The confirmed protective proteins were grouped into three sub-groups according to their correlation coefficients with each other (FIG. 5A). Sub-group (A) contained 5 extremely highly inter-correlated proteins; SPARC, CCL5, APP, PF4 and ANGPT1. Sub-group (B) contained 2 proteins; DNAJC19 and TNFSF12, that were moderately correlated between themselves and with proteins in sub-group (A). Sub-group (C) contained FGF20, a protein not correlated with any of the other proteins except for moderate correlation with TNFSF12. This pattern of grouping of proteins was preserved and confirmed in the hierarchical cluster analysis, as described in FIG. 5B. This finding suggests that plasma concentration of these three sub-groups of proteins are regulated by different mechanisms. This is in contrast to the 5 proteins in sub-group (A) which showed such strong inter-correlation that one can hypothesize that they are regulated by the same mechanisms.

To further test which of these 8 proteins (three sub-groups) independently contributed to protection against progressive renal decline, a multivariable logistic regression analysis was performed with backward elimination of proteins and clinical covariates that had no or weak effects (α>0.1) (Table 6). All relevant clinical characteristics and 8 confirmed protective proteins were included in the analysis. In the final model, three baseline clinical variables, eGFR, HbA1c, and ACR significantly increased the risk of progressive renal decline, and three baseline plasma proteins, ANGPT1 (exemplar of sub-group A), TNFSF12 (exemplar of sub-group B) and FGF20 (sub-group C) significantly protected against progressive renal decline. The odds ratios (95% CI) obtained from the multivariable logistic regression analysis for the clinical covariates and the exemplar protective proteins are shown in FIG. 5C.

TABLE 6
Ranking of proteins/clinical covariates for elimination from
the multivariable logistic regression analysis using backward
elimination procedure. Proteins with α > 0.1 were
eliminated from the final logistic regression model.

| | |
|---|---|
| Proteins/Clinical | Summary of backward elimination |

| | | |
|---|---|---|
| covariates | Wald Chi-Square | P-value |

Eliminated proteins

| | | |
|---|---|---|
| PF4 | 0.073 | 0.79 |
| SPARC | 0.18 | 0.67 |
| CCL5 | 0.58 | 0.45 |
| APP | 0.57 | 0.45 |
| DNAJC19 | 0.76 | 0.39 |

Selected proteins/covariates in the final model

| | | |
|---|---|---|
| eGFR | 5.21 | 0.022 |
| HbA1c | 8.43 | 0.0037 |
| ACR | 35.81 | <.0001 |
| TNFSF12 | 2.84 | 0.092 |
| ANGPT1 | 5.73 | 0.017 |
| FGF20 | 6.48 | 0.011 |

### Example 4. Combined Effect of Three Exemplar Protective Proteins

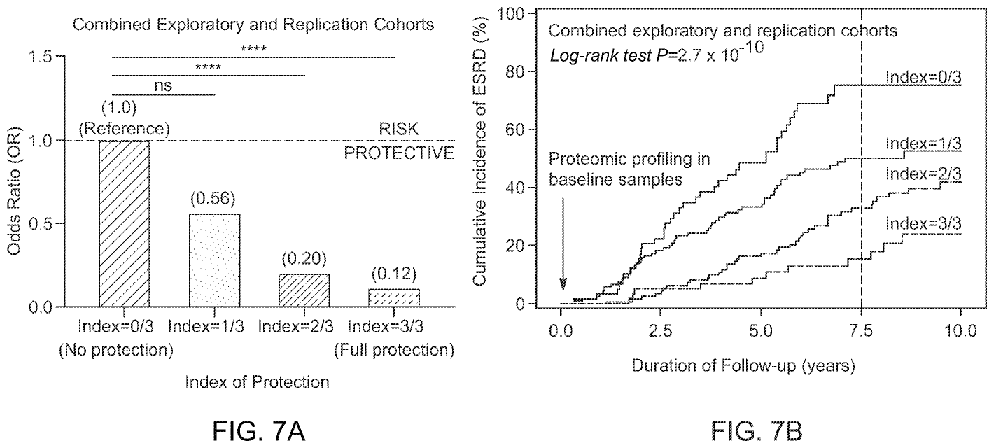

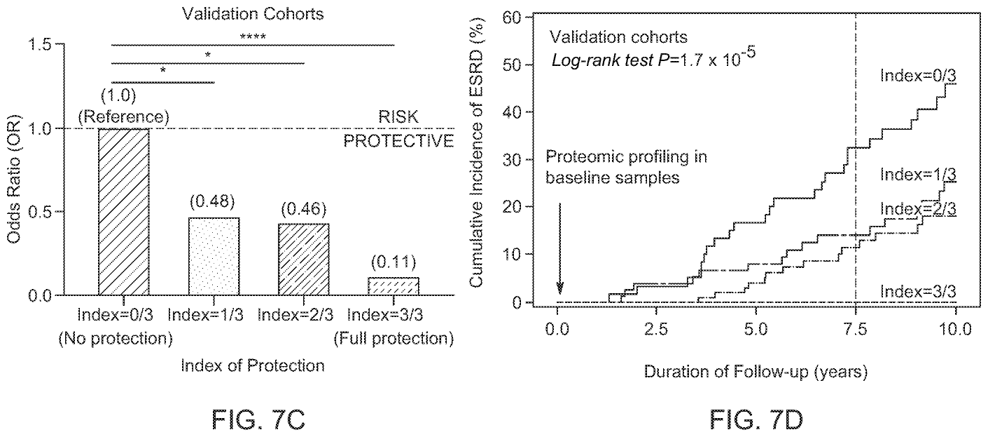

To estimate the combined effect of the three exemplar protective proteins on risk of progressive renal decline and progression to ESKD, an “index of protection” was developed. The plasma concentration of the three exemplar protective proteins (ANGPT1, TNFSF12 and FGF20) were evaluated in each subject. Value above median for each protein was scored as 1 and below as 0; by summing up the scores, a subject could have a total protection index varying between 0 (all proteins below median) and 3 (all proteins above median). The association between the index of protection and progressive renal decline is shown in FIG. 7A. The odds ratio (95% CI) for progressive renal decline was 0.69 (0.28, 1.69), 0.34 (0.14, 0.83) and 0.19 (0.1, 0.52) for subjects with the total index of protection 1, 2 and 3, respectively, when compared with subjects with the protection index value 0. To visualize the combined effect of the three protective proteins, the cumulative risk of progression to ESKD was analyzed in the combined study cohorts according to the index of protection. FIG. 7B shows the cumulative incidence of ESKD during 7.5 years of follow-up according to values of the protection index. Subjects with all 3 protective protein values above median had very low risk of developing ESKD, with the cumulative incidence of 16% during 7.5 years of follow-up. In contrast, those with the protective index value 0, e.g. all three protective protein values below median, had very high cumulative incidence of ESKD of 80%. The highly statistically significant P-value (P=2.7×10⁻¹⁰) indicates strong evidence of a significant difference in the cumulative incidence of ESKD among the four subgroups.

To examine whether the results shown in FIG. 7A could have been confounded by inflammatory circulating proteins (e.g. high TNF-R1 plasma concentration) or clinical covariates, the logistic regression analysis was performed in the combined Joslin cohorts (T1D and T2D). In this analysis, the protection index was considered as a continuous variable as opposed to discrete variable as in FIG. 7A. As shown in Table 7, the effect of index of protection was highly significant (P<0.0001), the odds ratio was 0.47 (95% CI:0.32-0.60). By including into the model one inflammatory protein, TNF-R1, reported by us previously (5), the protective effect of the index was attenuated, the odds ratio increased to 0.60 (95% CI:0.45-0.78) but remained highly statistically significant (P<0.0002). It is instructive that adding into the model many clinical covariates did not substantially change the odds ratio for the protective index.

TABLE 7
Effect estimates measured as odds ratios (95% CI) of index of protection (FGF20,
TNFSF12 and ANGPT1) on risk of progressive renal decline in univariate
and multivariable logistic regression models in both Joslin cohorts combined.

| | | | | Model comparisons | | |
|---|---|---|---|---|---|---|
| | Model | | | (P-value) | | |
| Predictive metrics | 1 | 2 | 3 | 2 vs 1 | 3 vs 2 | 3 vs 1 |
| C-statistics ± SE | 0.687 ± 0.03 | 0.765 ± 0.03 | 0.833 ± 0.02 | 0.0005 | <0.0001 | <0.0001 |
| −2 Log Likelihood | 439 | 401 | 352 | | | |
| Akaike information | 443 | 407 | 364 | | | |
| criterion (AIC) | | | | | | |
| Covariates | Odds Ratio (95% CI) | | | Significance (P-value) | | |
| Protection Index | 0.47 (0.36, | 0.60 (0.45, | 0.61 (0.45, | <0.0001 | 0.0002 | 0.001 |
| | 0.60) | 0.78) | 0.82) | | | |
| TNF-R1 | | 2.04 (1.61, | 1.63 (1.23, | | <0.0001 | 0.0007 |
| | | 2.58) | 2.15) | | | |
| HbA1c | | | 1.32 (1.12, | | | 0.001 |
| | | | 1.56) | | | |
| ACR | | | 2.54 (1.77, | | | <0.0001 |
| | | | 3.63) | | | |
| eGFR | | | 0.99 (0.96, | | | 0.49 |
| | | | 1.02) | | | |

SE, Standard error;
CI, Confidence intervals;
TNF-R1, Tumor necrosis factor receptor 1;
HbA1c, Hemoglobin A1c;
ACR, Albumin-to-creatinine ratio;
eGFR, Estimated glomerular filtration rate.
Model 1 has been compared to the model with the same protection index in the presence of TNF-R1 (Model 2) and to the model with same protection index and TNF-R1, in the presence of important clinical covariates (Model 3).

### Example 5. Validation of Three Exemplar Protective Proteins in Early CKD

To demonstrate the robustness of the findings, a validation study was conducted in an independent Joslin cohort of 294 subjects with T1D who had had albuminuria but normal kidney function at baseline. This cohort was followed for 7-15 years to determine eGFR slope and ascertain time of onset of ESKD. Plasma samples from the validation study of 294 T1D subjects underwent profiling of the proteins of interest using the same SOMAscan platform. In contrast to the exploratory and replication cohorts, which had impaired kidney function (CKD Stage 3) at baseline, the validation cohort had normal kidney function (CKD Stages 1 and 2; Median eGFR (25^{th}, 75^{th} percentile): 100 (82, 114) ml/min/1.73 m²) at baseline. The clinical characteristics of the validation cohort are shown in Table 8.

TABLE 8
Demographics and clinical characteristics of an independent validation
cohort of T1D subjects with normal kidney function.

| | Validation Cohort | |
|---|---|---|
| | Joslin T1D CKD12 Cohort | |
| Characteristics | (N = 294) | |
| At baseline | | |
| Male (%) | 55 | |
| Age (years) | 38 | (32, 45) |
| Duration of diabetes (years) | 25 | (17, 32) |
| HbA1c (%) | 8.8 | (7.9, 9.8) |
| eGFR (ml/min/1.73 m²) | 100 | (82, 114) |
| ACR (μg/mg creatinine) | 491 | (112, 1099) |
| During follow-up | | |
| eGFR slope (ml/min/1.73 m²/year) | −2.6 | (−7.1, −1.1) |

| | |
|---|---|
| Non-progressors* (%) | 53 |
| Progressors* (%) | 47 |
| New cases of ESKD within 10 years follow-up (%) | 19 |

T1D, Type 1 diabetes; CKD, Chronic Kidney Disease; HbA1c, Hemoglobin A1c; eGFR, Estimated glomerular filtration rate; ACR, Albumin-to-creatinine ratio; ESKD, End-stage renal disease. Non-progressors were defined as eGFR loss <3.0 ml/min/1.73 m²/year and progressors as eGFR loss ≥3.0 ml/min/1.73 m²/year. Data presented as median (25th, 75th percentile) or count (proportion) measures.

The plasma concentration of the three exemplar protective proteins (ANGPT1, TNFSF12 and FGF20) were evaluated in each subject and the index of protection was developed. The association between the index of protection and progressive renal decline is shown in FIG. 7C. The odds ratio (95% CI) for progressive renal decline was 0.48 (0.24, 0.95), 0.46 (0.24, 0.89) and 0.11 (0.05, 0.27) for subjects with the total index of protection 1, 2 and 3, respectively, when compared with subjects with the protection index value 0. The cumulative risk of progression to ESKD was also analyzed in the validation cohort according to the index of protection. FIG. 7D shows the cumulative incidence of ESKD during 7.5 years of follow-up according to values of the protection index. None of the subjects with all 3 protective protein values above median progressed to ESKD during 7.5 years of follow-up. The low cumulative incidence of ESKD was observed for subjects with the protection index values 1 and 2; 14% and 11%, respectively, when compared with subjects with the protection index value 0 with the cumulative incidence of 33% during 7.5 years of follow-up. The highly statistically significant P-value (P=1.7×10⁻⁵) suggests strong evidence of a significant difference in the cumulative incidence of ESKD among the four subgroups.

Furthermore, two (ANGPT1 and FGF20) out of three exemplar protective proteins were validated using different platforms. ANGPT1 measurements were validated in a subset of samples (n=32) using the Human Ang-1 MSD R-Plex assay (F21YQ-3, Meso Scale Diagnostics) according to the manufacturer's protocols. Briefly, an MSD GOLD Small Spot Streptavidin plate was coated with 100 μl of biotinylated Ang-1 capture antibody in coating diluent 100 and incubated for 1 hour at room temperature. The plate was washed with 150 μl/well of washing buffer (1×PBS-Tween 20), and duplicates of 25 μl of serially diluted standard from 100,000 pg to 24 pg/ml and 32 plasma samples from our study were all loaded on the same plate. After 1-hour incubation with shaking at room temperature, the plate was washed and incubated with 50 μl of conjugated detection antibody (MSD GOLD SULFO-TAG™) for 1 hour at room temperature, then washed, and finally 150 μl/well of read buffer was added on the plate. The plate was loaded into an MSD instrument where a voltage was applied to the plate electrodes to measure to intensity of the emitted light and provided a quantitative measure of the analyte in the sample.

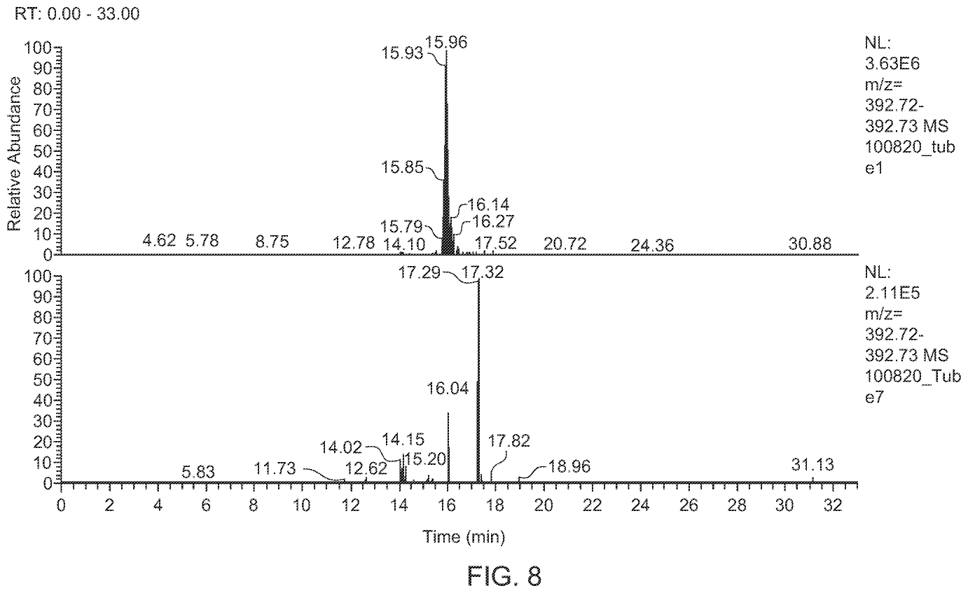

The correlation between antibody-based (MSD) measurements and aptamer-based (SOMAscan) results was extremely good. The Spearman's rank correlation coefficient between the SOMAscan and MSD ANGPT1 measurements was rₛ=0.76, P<0.0001. To analytically validate SOMAmer specificity, protocols integrating DNA-based affinity pull-down of intact proteins with mass spectrometry were developed. Fourteen FGF20 tryptic peptides spanning amino acids (a.a.) 50-211 of the FGF20 protein sequence were identified in the FGF20 SOMAmer plasma pull-downs spiked with recombinant FGF20, whereas no FGF20 peptides were identified in the FGF20 SOMAmer plasma pull-downs that were not spiked with recombinant FGF20. An example of an extracted ion chromatogram of FGF20 tryptic peptide GGPGAAQLAHLHGILR (a.a. 50-65; SEQ ID NO: 9) is shown in FIG. 8. This FGF20 peptide was identified in the plasma pull-down spiked with recombinant FGF20 but was not detected in the plasma pull-down not spiked with recombinant FGF20, thereby verifying the FGF20 SOMAmer specificity on the SOMAscan platform.

### Example 6. Plasma Concentration of Protective Proteins in Non-Diabetic and Diabetic Subjects

Two possibilities exist on how to explain the elevated concentrations of protective proteins in non-progressors compared to those at risk of progressive renal decline at study entry. The first possibility is that diabetes and related kidney damage may cause a decrease in plasma concentrations of the putative protective proteins. As a result, progressors would have lower protein concentrations than non-progressors due to more extensive underlying kidney damage, which was not recognized by clinical covariates and not accounted for in the multivariable models. If this was true, one would hypothesize that protective proteins are further elevated in non-diabetics as compared to slow-declining diabetics. The second possibility is that diabetes may not be a factor in determining the concentrations of the putative protective proteins, however, elevated concentrations of these proteins at baseline could protect against progressive renal decline. Consequently, subjects with elevated plasma concentrations of these proteins would comprise mainly non-progressors, whereas those with low concentrations of these proteins would be at risk of progressive renal decline. If this was true, one would hypothesize that, in comparison with non-diabetics, non-progressors should have higher concentrations of the putative protective proteins, whereas progressors would have protein concentrations lower than or similar to the controls.

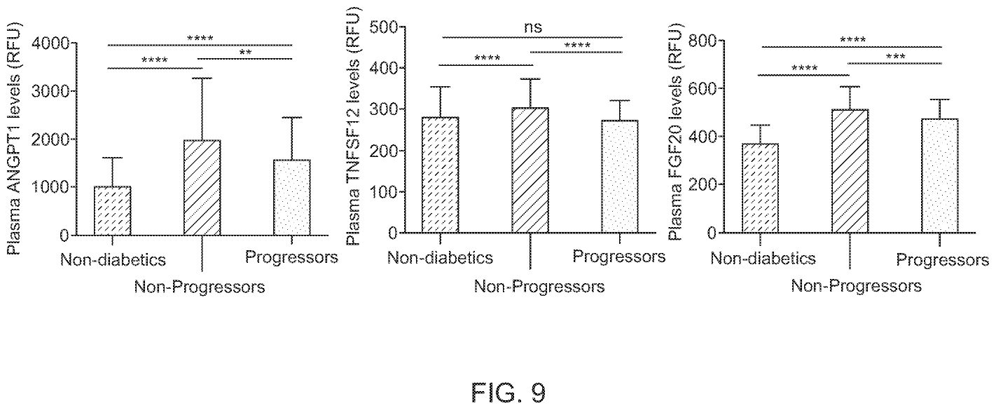

To distinguish between the two possibilities described above, plasma concentrations of the protective proteins were compared among healthy non-diabetic parents of T1D subjects, non-progressors and progressors with T1D and T2D, using the same aptamer-based SOMAscan platform. Baseline clinical characteristics and baseline values of the protective proteins among the three study sub-groups are shown in Table 9. The non-diabetics were older, had normal HbA1c, normal ACR and almost normal eGFR in comparison with diabetic subjects. By design, non-progressors and progressors had similarly impaired kidney function at baseline but dramatically different eGFR slopes during 7-15 years of follow-up. With regard to the 8 confirmed protective proteins, the lowest baseline concentrations were observed in non-diabetics and the highest values were observed in non-progressors, while progressors' concentrations fell between the two other sub-groups. A comparison of the 3 exemplar protective proteins among the 3 sub-groups is shown in FIG. 9, supporting the role of these protective proteins primarily against progressive renal decline.

TABLE 9
Clinical characteristics and plasma concentrations of 8 confirmed protective proteins in non-diabetic
parents of T1D subjects and in the combined Joslin cohorts, for non-progressors and progressors.

| | | | Combined Joslin cohorts (N = 358) | | | |
|---|---|---|---|---|---|---|
| | Non-diabetics | | Non-progressors | | Progressors | |
| Characteristics | (N = 79) | | (N = 140) | | (N = 218) | |
| At baseline | | | | | | |
| Male, n | 40 | (51%) | 78 | (56%) | 120 | (55%) |
| Age at study entry (years) | 61 | (57, 66) | 56 | (48, 61) | 47 | (40, 60) |
| Duration of diabetes (years) | — | | 24 | (14, 34) | 24 | (18, 31) |
| BMI (kg/m²) | — | | 29 | (25, 34) | 27 | (24, 33) |
| Systolic BP (mm Hg) | — | | 133 | (122, 148) | 136 | (126, 149) |
| Diastolic BP (mm Hg) | — | | 72 | (67, 81) | 78 | (70, 83) |
| Insulin Rx, % | — | | 81% | | 89% | |
| Renoprotection Rx, % | — | | 82% | | 83% | |
| HbA1c (%) | 5.4 | (5.2, 5.6) | 7.4 | (6.9, 8.6) | 8.4 | (7.4, 9.6) |
| eGFR (ml/min/1.73 m²) | 71.2 | (62, 82) | 49 | (42, 55) | 42 | (34, 51) |
| ACR (mg/g creatinine) | 5.8 | (3.9, 7.8) | 175 | (40, 502) | 1106 | (402, 2232) |
| During follow-up | | | | | | |

| | | | | | |
|---|---|---|---|---|---|
| eGFR slope (ml/min/1.73 m²/year) | — | −1.2 | (−2.2, −0.31) | −6.2 | (−9.8, −4.1) |
| Deaths unrelated to ESKD, n (%) | — | 10 | (7%) | 13 | (6%) |

Baseline plasma concentrations (RFU)
Sub-group A

| | | | | | | |
|---|---|---|---|---|---|---|
| SPARC | 17775 | (13587, 28777) | 43192 | (30001, 62701) | 33266 | (21572, 49352) |
| CCL5 | 14900 | (7180, 24835) | 25351 | (13498, 46022) | 18973 | (10635, 32056) |
| APP | 23162 | (17824, 36917) | 45776 | (29392, 72317) | 35561 | (23106, 52307) |
| PF4 | 20031 | (9581, 46044) | 52730 | (21260, 100893) | 31230 | (13449, 70052) |
| ANGPT1 | 757 | (640, 1189) | 1564 | (1093, 2522) | 1248 | (934, 1916) |

Sub-group B

| | | | | | | |
|---|---|---|---|---|---|---|
| DNAJC19 | 540 | (484, 585) | 587 | (540, 675) | 555 | (507, 604) |
| TNFSF12 | 270 | (240, 296) | 291 | (269, 316) | 267 | (244, 288) |

Sub-group C

| | | | | | | |
|---|---|---|---|---|---|---|
| FGF20 | 371 | (311, 417) | 491 | (449, 550) | 460 | (421, 507) |

BMI, Body mass index; BP, Blood pressure; Rx, treatment; Renoprotection, Prescription of angiotensin-converting enzyme inhibitor (ACE-I) or angiotensin II receptor blocker (ARB); HbA1c, Hemoglobin A1c; eGFR, Estimated glomerular filtration rate; ACR, Albumin-to-creatinine ratio; RFU, Relative fluorescence unit. Data presented as median (25^{th}, 75^{th} percentile) or count (proportion) measures.

To examine whether plasma concentration of protective proteins preceded the diabetic state and the development of early renal decline, a comparative analysis was performed on plasma concentration of the 3 exemplar protective proteins (ANGPT1, TNFSF12 and FGF20) in non-diabetic parents of two categories of T1D probands, normo-albuminuria or ESKD (or proteinuria). Baseline characteristics and baseline values of the 3 protective proteins among non-diabetic parents of the two categories of T1D probands are shown in Table 10. Interestingly, as depicted in Table 10, parents of children with kidney complications (ESKD or Proteinuria) had significantly lower circulating FGF20 concentrations than parents with children who remained without kidney complications despite long diabetes duration.

TABLE 10
Circulating plasma concentrations of top 3 protective proteins
in non-diabetic parents of two categories of T1D probands.

| | Normoalbuminuria | Proteinuria or ESKD |
|---|---|---|
| Characteristics | (N = 40) | (N = 39) |
| At baseline | | |
| Male, n (%) | 50% | 51% |
| Age, years | 61 ± 6 | 62 ± 5 |
| eGFR (ml/min/1.73 m²) | 75 ± 13 | 71 ± 13 |
| HbAlc (%) | 5.4 ± 0.3 | 5.4 ± 0.4 |
| ACR (μg/mg creatinine) | 5.9 ± 3.4 | 9.4 ± 13.5 |

Baseline plasma concentrations (RFU)

| | | | | |
|---|---|---|---|---|
| ANGPT1 | 771 | (577, 1185) | 746 | (658, 1203) |
| TNFSF12 | 266 | (242, 283) | 273 | (240, 303) |
| FGF20 | 392 | (351, 449) | 337 | (298, 383)** |

ESKD, end-stage kidney disease; HbA1c, hemoglobin A1c; ACR, albumin-to-creatinine ratio; eGFR, estimated glomerular filtration rate; RFU, relative fluorescent unit. Data presented as mean ± standard deviation, median (25^{th}, 75^{th} percentile) or count (proportion) measures. Differences between the two groups were tested using the Wilcoxon-rank-sum test for continuous variables.
**P < 0.01.

### Discussion of Examples 1 to 6

Through unbiased proteomic profiling, the present study described in the above examples identified circulating plasma proteins that were specifically associated with protection against progressive renal decline and progression to ESKD in two independent cohorts of subjects with diabetes and moderately impaired kidney function. Eight circulating proteins were identified that had a protective effect against progressive renal decline independent from clinical covariates such as baseline eGFR, HbA1c, ACR and type of diabetes. These proteins could be grouped into three sub-groups; (A) SPARC, CCL5, APP, PF4, ANGPT1, (B) DNAJC19, TNFSF12 and (C) FGF20. It is instructive to note that when the 8 confirmed protective proteins were considered together, only three proteins representing each of the sub-groups, e.g., ANGPT1, TNFSF12 and FGF20, showed a strong independent protective effect against progressive renal decline. The combined effect of these 3 exemplar protective proteins was nicely demonstrated by very low cumulative risk of ESKD in subjects who had values above median for all 3 proteins at the beginning of follow-up. Furthermore, the fact that the concentrations of these protective proteins were much higher in non-progressors than non-diabetics provides strong evidence that the proteins or the pathways that they represent, are causally involved in protection against progressive renal decline. These study findings are highly generalizable as the importance of these 3 exemplar protective proteins is confirmed in three independent cohorts of study participants with different types of diabetes, T1D and T2D, and at different stages of DKD, those with early and late stages of DKD, that were prospectively followed for a decade.

Angiopoietins (ANGPT) are growth factors involved in angiogenesis and vascular inflammation. Among the members of the ANGPT family, Angiopoietin-1 (ANGPT1) and Angiopoietin-2 (ANGPT2) are both ligands for the Tie-2 receptor (Suri et al., Cell 87: 1171-1180 (1996); Maisonpierre et al., Science 277: 55-60 (1997)). ANGPT1 is a major ligand and activator of the Tie-2 receptor, maintaining vessel integrity by activation of the phosphatidyl-inositol 3-kinase/protein kinase B (PI3K/Akt) pathway (Brindle et al., Circ Res 98: 1014-1023 (2006)), therefore protecting the endothelium from excessive activation by growth factors and cytokines (Fiedler et al., Trends Immunol 27: 552-558 (2006)). ANGPT2, on the other hand, is considered a natural antagonist of ANGPT1 by preventing the binding of ANGPT1 to the Tie-2 receptor, consequently reducing ANGPT1/Tie-2 pathway activation and promoting blood vessel wall destabilization and vascular leakage (Maisonpierre et al., Science 277: 55-60 (1997); Fiedler et al., Trends Immunol 27: 552-558 (2006)). Since ANGPT1 and ANGPT2 are competing with each other for the Tie-2 receptor and have opposite actions, it is perhaps beneficial to measure both angiopoietins to assess the equilibrium of the ongoing angiogenesis process, such that disruption of the equilibrium between ANGPT1 and ANGPT2 (e.g. in favor of ANGPT2) leads to diabetes-mediated angiopoietin imbalance, e.g. destabilization of blood vessel walls, promotes inflammation and fibrosis (Gnudi, Diabetologia 59: 1616-1620 (2016)). Since ANGPT2 was measured on the SOMAscan platform and the results were available for this study, the protective effect of ANGPT1 was compared with the risk effect of ANGPT2 as well as the effect of ratio of ANGPT1/ANGPT2 (in favor of ANGPT1) on the risk of progressive renal decline. Unfortunately, the findings of these analyses did not show a stronger protective effect of the ratio of the two angiopoietins in comparison with the protective effect of ANGPT1 alone (Table 11), supporting the protective role of ANGPT1 alone against progressive renal decline rather than the ratio of the two angiopoietins.

TABLE 11
Logistic regression models comparing the protective
effect of ANGPT1, the risk effect of ANGPT2 and the
effect of ANGPT1/ANGPT2 ratio on the risk of progressive
renal decline in the combined Joslin cohorts.

| | Model 1 | Model 2 |
|---|---|---|
| Protein | OR (95% CI) | OR (95% CI) |
| ANGPT1 | 0.68 (0.56, 0.83) | 0.72 (0.57, 0.91) |
| ANGPT2 | 1.48 (1.21, 1.81) | 1.19 (0.95, 1.51) |
| ANGPT1/ANGPT2 Ratio | 0.68 (0.55, 0.82) | 0.79 (0.63, 1.01) |

ANGPT1, Angiopoietin-1; ANGPT2, Angiopoietin-2. Model 1: Unadjusted; Model 2: Adjusted for baseline eGFR, HbA1c and ACR. All models were adjusted by type of diabetes.

More research has been done regarding the protective effect of ANGPT1. ANGPT1 has been shown to exert an anti-inflammatory effect and protect endothelial cell permeability against inflammatory factors (Pizurki et al., Br J Pharmacol 139: 329-336 (2003)). A variant of ANGPT1, known as Cartilage Oligomeric Matrix Protein-angiopoietin-1 (COMP-Ang1) was developed to investigate the protective effect of COMP-Ang1 in unilateral ureteral obstruction-induced renal fibrosis and in diabetic nephropathy animal models (Kim et al., J Am Soc Nephrol 17: 2474-2483 (2006); Lee et al., Nephrol Dial Transplant 22: 396-408 (2007)). Diabetic db/db mice treated with COMP-Ang1 had reduced albuminuria and fasting blood glucose concentrations, decreased mesangial expansion, thickening of the glomerular basement membrane and podocyte foot process broadening (Lee et al., Nephrol Dial Transplant 22: 396-408 (2007)). Studies using genetically modified mice have further confirmed the importance of ANGPT1 expression concentrations in diabetic glomerular disease. Overexpression or repletion of ANGPT1 in diabetic mice, specifically in the glomeruli, led to a reduction in albumin excretion accompanied by a decrease in diabetes-induced nephrin phosphorylation (Dessapt-Baradez et al., J Am Soc Nephrol 25: 33-42 (2014)), resulting in a reduced nephrin degradation and podocyte foot process broadening, leading to a more stable and functional glomerular filtration barrier (Zhu et al., Kidney International 73: 556-566 (2008)). Taking all these observations together with our strong findings in humans showing elevated plasma ANGPT1 concentrations protected against progressive renal decline, it is quite evident that ANGPT1 may be a potential therapeutic target to prevent or reduce the risk of progressive renal decline in diabetes.

The present study demonstrated that ANGPT1 is significantly and highly correlated with four other confirmed protective proteins (PF4, SPARC, APP and CCL5), suggesting that these proteins may have similar physiological relevance, be part of common pathways or be under the same genetic regulations. A common pathway in which all 5 of these proteins are expressed and secreted relates to platelet function. Thrombin is known to induce the release of ANGPT1 from platelets to aid in endothelial cell stabilization during vascular repair (Li et al., Thromb Haemost 85: 204-206 (2001)). Platelet Factor-4 (PF4) is released from the alpha-granules of activated platelets and binds with high affinity to heparin. It is a strong chemoattractant for neutrophils, fibroblasts, and monocytes (Lord et al., J Biol Chem 292: 4054-4063 (2017)). Secreted protein acidic and rich in cysteine (SPARC) is also an alpha granule component of human platelets and is secreted during platelet activation. Additionally, it is also produced by fibroblasts, endothelial cells, macrophages, and adipocytes. SPARC is involved in cell proliferation, repair of tissue damage, collagen matrix formation, and osteoblast differentiation (Yun et al., Biomed Res Int 2016: 9060143 (2016)). Platelets are the primary source of amyloid beta A4 protein (APP) in blood circulation (Li et al., Blood 84: 133-142 (1994)). C-C motif chemokine 5 (CCL-5), also known as RANTES, is also released by activated platelet alpha-granules, deposited on inflamed endothelium, and mediates transmigration of monocytes onto activated endothelium. Low plasma CCL-5 concentrations are an independent predictor of cardiac mortality in patients referred for coronary angiography (Nomura et al., Clin Exp Immunol 121: 437-443 (2000)). Previous studies have reported that activated platelets play a role in the development of diabetic nephropathy (Omoto et al., Nephron 81: 271-277 (1999); Zhang et al., J Am Soc Nephrol 29: 2671-2695 (2018)). The results of this study further point to the importance of platelet secreted proteins in the progression of diabetic nephropathy. Platelet activated protein secretion may protect against vascular damage associated with leukocyte trafficking, thereby protecting against faster progression of diabetic nephropathy. The relevance of these proteins with regard to protection against progressive renal decline needs to be investigated further.

Tumor Necrosis Factor (TNF) Ligand Superfamily Member 12 (TNFSF12), also known as TWEAK, is a member of a large TNF superfamily of ligands and receptors (Chicheportiche et al., J Biol Chem 272: 32401-32410 (1997)). Findings from in vitro and in vivo models have shown that the administration of TNFSF12 increases inflammatory cytokine production in renal tubular cells, e.g. increased mRNA and protein expression of monocyte chemoattractant protein-1 and interleukin-6 (IL-6), whereas the blockage of TNFSF12 prevented tubular chemokine and IL-6 expression, interstitial inflammation and macrophage infiltration in mice (Sanz et al., J Am Soc Nephrol 19: 695-703 (2008)). The role of TNFSF12 in the development/progression of DKD remains unclear. So far there has been sparse literature devoted to this topic; a few cross-sectional studies have investigated a relationship between circulating TNFSF12 concentrations and DKD. One study reported decreased circulating TNFSF12 concentrations in T2D and ESKD subjects (Kralisch et al., Atherosclerosis 199: 440-444 (2008)). The actions of TNFSF12 in other kidney diseases and other forms of diabetes have also been reported (Sanz et al., J Cell Mol Med 13: 3329-3342 (2009); Dereke et al., PLoS One 14: e0216728 (2019); Bernardi et al., Clin Sci (Lond): 133, 1145-1166 (2019)). In experimental folic acid-induced acute kidney injury, TNFSF12 deficiency reduced kidney apoptosis and inflammation and improved kidney function. A case-control study involving women with and without gestational diabetes mellitus (GBM) reported decreased TNFSF12 concentrations in women with GBM compared to pregnant volunteers without GBM. The present study is the only follow-up observation in which very robust findings point to TNFSF12 as a protective protein against progressive renal decline, contrary to findings in the aforementioned studies. This finding needs to be explored further in humans and in animal studies.

Fibroblast growth factor 20 (FGF20) is a member of a large family of 22 fibroblast growth factors (FGFs), comprising 7 sub-families consisted of secreted signaling proteins and intracellular non-signaling proteins (Itoh et al., J Biochem 149: 121-130 (2011)). Seventeen out of 22 FGFs were measured on the SOMAscan proteomic platform and only FGF20 was robustly associated with protection against progressive renal decline. FGF20 is a novel neurotrophic factor that was originally identified in the rat brain and has been suggested to play vital roles in the development of dopaminergic neurons (Ohmachi et al., Biochem Biophys Res Commun 277: 355-360 (2000); Correia et al., Front Neuroanat 1: 4 (2007); Shimada et al., J Biosci Bioeng 107: 447-454 (2009)). In addition, numerous studies have reported correlations between Parkinson's disease susceptibility with FGF20 genetic polymorphisms in different ethnicities although some studies reported no evidence of association between FGF20 and Parkinson's disease (Pan et al., Parkinsonism Relat Disord 18: 629-631 (2012); Sadhukhan et al., Neurosci Lett 675: 68-73 (2018); van der Walt et al., Am J Hum Genet 74: 1121-1127 (2004); Clarimon et al., BMC Neurol 5: 11 (2005); Wider et al., Mov Disord 24: 455-459 (2009)). Interestingly, a previous study demonstrated the essential role of FGF20/Fgf20 in the development of kidney by maintaining the stemness of nephron progenitors both in humans and in mice (Barak et al., Dev Cell 22: 1191-1207 (2012)). FGF20 was expressed exclusively in nephron progenitors in the kidney. Loss of FGF20/Fgf20 in humans and in mice resulted in kidney agenesis, a condition in which one or both fetal kidneys fail to develop and hence a newborn was missing one or both kidneys.

FGF20 was first discovered in 2001 by Jeffers and his colleagues as they identified FGF20 as a novel oncogene that may represent a potential target for the treatment of human malignancy (Jeffers et al., Cancer Research 61: 3131-3138 (2001)). Subsequently, the same authors demonstrated that FGF-20 (CG53135-05) has therapeutic activity to treat experimental intestinal inflammation (Jeffers et al., Gastroenterology 123: 1151-1162 (2002)), whereas another study reported FGF20 as a novel radioprotectant such that the administration of a single dose of FGF20 in mice before potentially lethal total-body radioactivity, reduced the lethal effects of acute radiation exposure and led to substantial increases in overall survival (Maclachlan et al., Int J Radiat Biol 81: 567-579 (2005)). Based on these findings, CG53135-05 (re-named as Velafermin) was evaluated in a Phase II clinical trial of cancer patients as a protective drug against developing oral mucositis, a side effect of chemotherapy (Schuster et al., Support Care Cancer 16: 477-483 (2008)). Results of this trial showed that Velafermin had a favorable safety and tolerability profile, however, it did not demonstrate sufficient efficacy when added to the treatment of oral mucositis.

The present study demonstrates FGF20 as one of the confirmed protective proteins that is most strongly associated with protection against progressive renal decline and progression to ESKD in the combined cohorts with T1D and T2D. The association is independent from circulating inflammatory proteins and relevant clinical covariates. High plasma concentrations of FGF20 at baseline predicted less renal decline during 7-15 years of follow-up. This association points to the involvement of FGF20 and its independent role to retard or decrease the risk of progressive renal decline and development of ESKD. As such, FGF20 may be a useful target for preventing or delaying the onset of progressive renal decline and ESKD in diabetes. Another interesting finding from our study was observed in plasma profiles of non-diabetic parents of two categories of T1D probands, either normo-albuminuria or ESKD/Proteinuria. Surprisingly, non-diabetic parents of T1D offspring with ESKD/Proteinuria had significantly lower plasma concentrations of FGF20 than those parents with T1D offspring without kidney complications. These findings prompt a question and/or speculation whether a genetic predisposition or component inherited from a parent may modulate corresponding protein concentrations in their offspring, and if confirmed in larger studies, could have a profound implication in future research on determinants of progressive renal decline in T1D (and also in T2D).

Recent interest in studies on protective factors against late diabetic complications, including DKD, has been initiated by the Joslin Medalist Study. This cross-sectional study enrolled nationwide subjects who survived with T1D for at least 50 years. Those who remained without late diabetic complications have been compared with regard to a large number of characteristics including various—omics profiles of biospecimens with non-diabetic spouses and with those who developed complications very late in the diabetes course. Comparing proteomic profiles of kidney tissues obtained from subjects in the three sub-groups, several glucose metabolic enzymes/proteins were identified in the glomeruli, including PKM2, which were highly elevated among those who remained without DKD despite extremely long duration of diabetes. By following this finding with a series of functional studies, the authors concluded that the upregulation of PKM2 may be a way of preventing the development of DKD (Qi et al., Nat Med 23: 753-762 (2017)).

The present study also searched for protective factors but was very different from the Medalist study. Where the latter was cross-sectional and searched for candidate protective proteins to be investigated in cellular and animal studies, this study was a Joslin clinic population-based prospective observation that investigated the association between baseline circulating plasma proteins that protected against progressive renal decline and fast progression to ESKD during 7-15 years of follow-up. Furthermore, the two studies were based on two different premises. The Medalist study aimed to find protective proteins against onset/development of late diabetic complications whereas this study aimed to identify protective proteins against progressive renal decline in subjects with already existing mild renal impairment. This is most likely the reason we could not confirm with statistical significance the PKM2 finding obtained in the Joslin Medalist study.

The strengths of this study include its prospective design, long-term follow-up observations of three independent study cohorts, the consistency of data in T1D and T2D, and the use of SOMAscan proteomic platform to measure protein concentrations in all Joslin cohorts. Furthermore, in this study, findings for key potential confounders and type of diabetes were adjusted. However, as with any study, the present study must be also considered in light of potential limitations. First, this is an observational study and while these proteins might directly protect against progression of renal decline, they could alternatively be indirect reporters of a protective process. Causal explanations of our findings will need to be established through animal models and clinical trials for confirmation that they are directly protective. Second, the findings are restricted to Caucasian individuals with diabetes who have chronic kidney disease and impaired kidney function, therefore, the results may not be generalizable to individuals in other populations and with other kidney diseases. Third, the baseline plasma samples were not taken at the onset of diabetes, hence, slow or fast progressive renal decline is relative to the time of blood sampling but not the onset of disease. The present study includes a subset of participants enrolled into the JKS in the 2000s and followed until 2012-13. Before enrollment, these individuals were under the care of the Joslin Clinic for many years (it was impractical to follow these individuals at the very beginning of diabetes onset) and their inclusion in our prospective studies was unrelated to their unknown future outcomes during subsequent follow-up. Therefore, these study findings reflect the unbiased contemporary natural history of CKD and the development of ESKD in individuals with diabetes. Notwithstanding the foregoing, the identification of protective proteins for ESKD and progression thereto, is remarkable and provides ample opportunity for both diagnostics and therapies for addressing what is a devastating diagnosis for any patient.

### Example 7. Circulating Level of Testican-2 is Independently Associated with Protection Against ESKD in T1D Patients

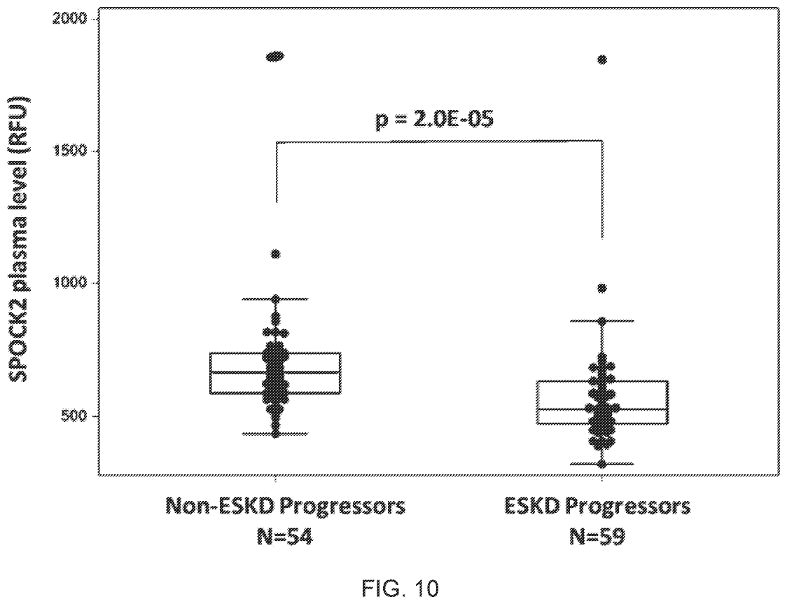

We searched for additional protective proteins using SOMAscan in a small Joslin Cohort with T1D. Characteristics for patients who progressed to ESKD within 10 years of follow-up and for those who remained without ESKD are shown in Table 12. The circulating level of Testican-2 (SPOCK2) was significantly higher in non-progressors than in progressors. This difference is illustrated in FIG. 10. Similar difference was observed for the three protective proteins, FGF20, TNFSF12 and ANGPT1 as described in the examples above.

TABLE 12
Clinical characteristics of 113 Joslin T1D Late DKD patients.

| | | | |
|---|---|---|---|
| | Non-ESKD | | |
| | progressors | ESKD | |
| Characteristics | (n = 54) | progressors (n = 59) | p-value |

At baseline

| | | | | |
|---|---|---|---|---|
| Male, n (%) | 23 | (43%) | 31 | (53%) |
| Age (years) | 50 | (41, 56) | 45 | (37, 51) |
| Duration of diabetes (years) | 35 | (24, 41) | 28 | (21, 35) |
| HbAlc (%) | 8.3 | (7.5, 9.2) | 8.7 | (7.7, 10) |
| eGFR (ml/min/1.73 m⁻²) | 48 | (40, 53) | 36 | (25, 44) |
| ACR (mg/g creatinine) | 282 | (28, 681) | 1720 | (712, 2568) |

During follow-up

| | | |
|---|---|---|
| eGFR slope (ml/min/1.73 m⁻²/year) | −2.0 (−3.5, −1.0) | −6.7 (−10, −4.0) |

Baseline plasma concentrations (RFU)

| | | | | | |
|---|---|---|---|---|---|
| SPOCK2 | 664 | (588, 738) | 526 | (473, 635) | 2.04E−05 |
| FGF20 | 486 | (434, 531) | 432 | (392, 483) | 5.71E−05 |
| TNFSF12 | 283 | (261, 303) | 248 | (233, 270) | 2.04E−05 |
| ANGPT1 | 1866 | (1273, 2847) | 1272 | (1069, 1781) | 1.04E−03 |

T1D, Type 1 diabetes; DKD, Diabetic kidney disease; HbA1c, Hemoglobin A1c; eGFR, Estimated glomerular filtration rate; ACR, Albumin-to-creatinine ratio; ESKD, End-stage kidney disease; RFU, Relative fluorescence unit; SPOCK2, Testican-2; FGF20, Fibroblast growth factor 20; TNFSF12, Tumor necrosis factor superfamily ligand 12; ANGPT1, Angiopoietin-1.

To test the protective effect of circulating SPOCK2 (Testican-2) against progression to ESKD, we performed logistic a regression analysis. The results are shown in Table 13 below. In the univariate logistic regression (Model 1) all protective proteins had strong protective effect against progression to ESKD (OR below 1 indicates protective effect). Protective effect for SPOCK2 (Testican-2) was also seen in multivariable logistic regression analysis (Model 2) when relevant clinical variable and all protective proteins were analyzed together.

In conclusion, circulating level of SPOCK2 (Testican-2) is independently associated with protection against ESKD, and can be used together with the three protective proteins (FGF20, ANG1 and TNFSF12) previously reported to develop a so-called “protection index”.

TABLE 13
Associations of 4 protective proteins with the development
of ESKD in the Joslin cohort with T1D.

| | Logistic models | | | |
|---|---|---|---|---|
| | Model 1 | | Model 2 | |
| Protein | OR (95% CI) | P-value | OR (95% CI) | P-value |
| SPOCK2 | 0.37 (0.25, 0.57) | 3.20E−06 | 0.49 (0.30, 0.78) | 3.00E−04 |
| FGF20 | 0.49 (0.34, 0.72) | 2.00E−04 | x | x |
| TNFSF12 | 0.42 (0.29, 0.63) | 2.00E−05 | x | x |
| ANGPT1 | 0.57 (0.40, 0.81) | 2.00E−03 | x | x |
| eGFR | 0.37 (0.25, 0.57) | 3.20E−06 | x | x |
| ACR | 3.52 (2.21, 5.62) | 1.30E−07 | x | x |
| HbA1c | 1.38 (0.98, 1.96) | 6.75E−02 | x | x |

The effect is shown as an odds ratio (OR) per one quartile change in circulating concentration of specific protein with corresponding 95% CIs. OR below 1 indicates protection.

Model 1: OR for covariates without adjustments

Model 2; OR for SPOCK2 was adjusted for FGF20, ANG1, TNFSF12, eGFR, ACR and HbA1c

T1D, Type 1 diabetes; ESKD, End-stage kidney disease; OR, Odds ratio; CI, Confidence interval; HbA1c, Hemoglobin A1c; GFR, Glomerular filtration rate; ACR, Albumin-to-creatinine ratio, SPOCK2, Testican-2; FGF20, Fibroblast growth factor 20; TNFSF12, Tumor necrosis factor superfamily ligand 12; ANGPT1, Angiopoietin-1.

x: data not available

TABLE 14
SEQUENCE TABLE

| Sequence | | |
|---|---|---|
| Identifier | Amino Acid Sequence | Description |
| SEQ ID NO: 1 | MRAWIFFLLCLAGRALAAPQQEALPDETEVVEETVAE | Human |
| | VTEVSVGANPVQVEVGEFDDGAEETEEEVVAENPCQN | SPARC |
| | HHCKHGKVCELDENNTPMCVCQDPTSCPAPIGEFEKV | |
| | CSNDNKTFDSSCHFFATKCTLEGTKKGHKLHLDYIGPC | |
| | KYIPPCLDSELTEFPLRMRDWLKNVLVTLYERDEDNNL | |
| | LTEKQKLRVKKIHENEKRLEAGDHPVELLARDFEKNY | |
| | NMYIFPVHWQFGQLDQHPIDGYLSHTELAPLRAPLIPM | |
| | EHCTTRFFETCDLDNDKYIALDEWAGCFGIKQKDIDKD | |
| | LVI | |
| SEQ ID NO: 2 | MKVSAAALAVILIATALCAPASASPYSSDTTPCCFAYIA | Human |
| | RPLPRAHIKEYFYTSGKCSNPAVVFVTRKNRQVCANPE | CCL5 |
| | KKWVREYINSLEMS | |
| SEQ ID NO: 3 | MLPGLALLLLAAWTARALEVPTDGNAGLLAEPQIAMF | Human APP |
| | CGRLNMHMNVQNGKWDSDPSGTKTCIDTKEGILQYC | |
| | QEVYPELQITNVVEANQPVTIQNWCKRGRKQCKTHPH | |
| | FVIPYRCLVGEFVSDALLVPDKCKFLHQERMDVCETHL | |
| | HWHTVAKETCSEKSTNLHDYGMLLPCGIDKFRGVEFV | |
| | CCPLAEESDNVDSADAEEDDSDVWWGGADTDYADGS | |
| | EDKVVEVAEEEEVAEVEEEEADDDEDDEDGDEVEEEA | |
| | EEPYEEATERTTSIATTTTTTTESVEEVVREVCSEQAET | |
| | GPCRAMISRWYFDVTEGKCAPFFYGGCGGNRNNFDTE | |
| | EYCMAVCGSAMSQSLLKTTQEPLARDPVKLPTTAASTP | |
| | DAVDKYLETPGDENEHAHFQKAKERLEAKHRERMSQ | |
| | VMREWEEAERQAKNLPKADKKAVIQHFQEKVESLEQE | |
| | AANERQQLVETHMARVEAMLNDRRRLALENYITALQ | |
| | AVPPRPRHVFNMLKKYVRAEQKDRQHTLKHFEHVRM | |
| | VDPKKAAQIRSQVMTHLRVIYERMNQSLSLLYNVPAV | |
| | AEEIQDEVDELLQKEQNYSDDVLANMISEPRISYGNDA | |
| | LMPSLTETKTTVELLPVNGEFSLDDLQPWHSFGADSVP | |
| | ANTENEVEPVDARPAADRGLTTRPGSGLTNIKTEEISEV | |
| | KMDAEFRHDSGYEVHHQKLVFFAEDVGSNKGAIIGLM | |
| | VGGVVIATVIVITLVMLKKKQYTSIHHGVVEVDAAVTP | |
| | EERHLSKMQQNGYENPTYKFFEQMQN | |
| SEQ ID NO: 4 | MSSAAGFCASRPGLLFLGLLLLPLVVAFASAEAEEDGD | Human PF4 |
| | LQCLCVKTTSQVRPRHITSLEVIKAGPHCPTAQLIATLK | |
| | NGRKICLDLQAPLYKKIIKKLLES | |
| SEQ ID NO: 5 | MASTVVAVGLTIAAAGFAGRYVLQAMKHMEPQVKQV | Human |
| | FQSLPKSAFSGGYYRGGFEPKMTKREAALILGVSPTAN | DNAJC19 |
| | KGKIRDAHRRIMLLNHPDKGGSPYIAAKINEAKDLLEG | |
| | QAKK | |
| SEQ ID NO: 6 | MTVFLSFAFLAAILTHIGCSNQRRSPENSGRRYNRIQHG | Human |
| | QCAYTFILPEHDGNCRESTTDQYNTNALQRDAPHVEPD | ANGPT1 |
| | FSSQKLQHLEHVMENYTQWLQKLENYIVENMKSEMA | |
| | QIQQNAVQNHTATMLEIGTSLLSQTAEQTRKLTDVETQ | |
| | VLNQTSRLEIQLLENSLSTYKLEKQLLQQTNEILKIHEK | |
| | NSLLEHKILEMEGKHKEELDTLKEEKENLQGLVTRQTY | |
| | IIQELEKQLNRATTNNSVLQKQQLELMDTVHNLVNLCT | |
| | KEGVLLKGGKREEEKPFRDCADVYQAGENKSGIYTIYI | |
| | NNMPEPKKVFCNMDVNGGGWTVIQHREDGSLDFQRG | |
| | WKEYKMGFGNPSGEYWLGNEFIFAITSQRQYMLRIEL | |
| | MDWEGNRAYSQYDRFHIGNEKQNYRLYLKGHTGTAG | |
| | KQSSLILHGADFSTKDADNDNCMCKCALMLTGGWWF | |
| | DACGPSNLNGMFYTAGQNHGKLNGIKWHYFKGPSYSL | |
| | RSTTMMIRPLDF | |
| SEQ ID NO: 7 | MAARRSQRRRGRRGEPGTALLVPLALGLGLALACLGL | Human |
| | LLAVVSLGSRASLSAQEPAQEELVAEEDQDPSELNPQT | TNFSF12 |
| | EESQDPAPFLNRLVRPRRSAPKGRKTRARRAIAAHYEV | |
| | HPRPGQDGAQAGVDGTVSGWEEARINSSSPLRYNRQIG | |
| | EFIVTRAGLYYLYCQVHFDEGKAVYLKLDLLVDGVLA | |
| | LRCLEEFSATAASSLGPQLRLCQVSGLLALRPGSSLRIR | |
| | TLPWAHLKAAPFLTYFGLFQVH | |
| SEQ ID NO: 8 | MAPLAEVGGFLGGLEGLGQQVGSHFLLPPAGERPPLLG | Human |
| | ERRSAAERSARGGPGAAQLAHLHGILRRRQLYCRTGF | FGF20 |
| | HLQILPDGSVQGTRQDHSLFGILEFISVAVGLVSIRGVD | |
| | SGLYLGMNDKGELYGSEKLTSECIFREQFEENWYNTYS | |
| | SNIYKHGDTGRRYFVALNKDGTPRDGARSKRHQKFTH | |
| | FLPRPVDPERVPELYKDLLMYT | |
| SEQ ID NO: 9 | GGPGAAQLAHLHGILR | FGF20 |
| | | tryptic |
| | | peptide (a.a. |
| | | 50-65) |
| SEQ ID NO: | HHHHHH | hexahistidine |
| 10 | | |
| SEQ ID NO: | MRAPGCGRLVLPLLLLAAAALAEGDAKGLKEGETPGN | Human |
| 11 | FMEDEQWLSSISQYSGKIKHWNRFRDEVEDDYIKSWE | Testican-2 |
| | DNQQGDEALDTTKDPCQKVKCSRHKVCIAQGYQRAM | (SPOCK2) |
| | CISRKKLEHRIKQPTVKLHGNKDSICKPCHMAQLASVC | |
| | GSDGHTYSSVCKLEQQACLSSKQLAVRCEGPCPCPTEQ | |
| | AATSTADGKPETCTGQDLADLGDRLRDWFQLLHENSK | |
| | QNGSASSVAGPASGLDKSLGASCKDSIGWMFSKLDTS | |
| | ADLFLDQTELAAINLDKYEVCIRPFFNSCDTYKDGRVS | |
| | TAEWCFCFWREKPPCLAELERIQIQEAAKKKPGIFIPSC | |
| | DEDGYYRKMQCDQSSGDCWCVDQLGLELTGTRTHGS | |
| | PDCDDIVGFSGDFGSGVGWEDEEEKETEEAGEEAEEEE | |
| | GEAGEADDGGYIW | |

### INCORPORATION BY REFERENCE

The entire contents of all references, patents and published patent applications cited throughout this application are hereby incorporated by reference in their entirety.

1. A method of identifying a human subject at risk of developing progressive renal decline, said method comprising
detecting a level of at least one protective protein in a sample(s) from a subject in need thereof, wherein the protective protein is selected from the group consisting of fibroblast growth factor 20 (FGF20), angiopoietin-2 (ANGPT1), and tumor necrosis factor ligand superfamily member 12 (TNFSF12),
comparing the level of the protective protein with a reference level of the protective protein, wherein the reference level is a level of the protective protein in a non-progressor human subject,
wherein a lower level of the protective protein in comparison to the non-progressor reference level indicates that the human subject is at risk of developing progressive renal decline, or
wherein an equivalent or higher level of the protective protein in comparison to the reference level indicates that the human subject is not at risk of developing progressive renal decline.

2. The method of claim 1, wherein levels of a combination of protective proteins are detected,
wherein the combination of protective proteins is selected from the group consisting of FGF20 and TNFSF12; FGF20 and ANGPT1; and TNFSF12 and ANGPT1; or
wherein the combination of protective proteins includes FGF20, TNFSF12, and ANGPT1.

3. A method of identifying a human subject at risk of developing progressive renal decline, said method comprising
detecting a level of at least one protective protein in a sample(s) from a subject in need thereof, wherein the protective protein is selected from the group consisting of
a protective protein from a first group of protective proteins selected from the group consisting of Testican-2, secreted protein acidic and rich in cysteine (SPARC), C-C motif chemokine 5 (CCL5), amyloid beta A4 protein (APP), platelet factor-4 (PF4) and ANGPT1,
a protective protein from a second group of protective proteins selected from the group consisting of DNAJC19 and TNFSF12, and FGF20,
comparing the level of the protective protein with a reference level of the protective protein, wherein the reference level is a level of the protective protein in a non-progressor human subject,
wherein a lower level of the protective protein in comparison to the reference level indicates that the human subject is at risk of developing progressive renal decline, or
wherein an equivalent or higher level of the protective protein in comparison to the reference level indicates that the human subject is not at risk of developing progressive renal decline.

4. The method of claim 3, wherein levels of a combination of protective proteins are detected, wherein the combination of protective proteins is selected from the group consisting of FGF20 and a group 1 protective protein; FGF20 and a group 2 protective protein; a group 1 protective protein and a group 2 protective protein; and FGF20, a group 1 protective protein and a group 2 protective protein.

5. The method of claim 1, wherein the non-progressor is a non-diabetic human subject.

6. The method of claim 1, further comprising administering a therapy to improve kidney function if the subject is identified as having a risk for progressive renal decline; and/or further comprising administering to the subject FGF20, an active fragment of FGF20, an FGF20 mimic, or a nucleic acid encoding FGF20, or an active fragment thereof, if the subject is identified as having a risk for progressive renal decline; and/or further comprising administering to the subject ANGPT1, an active fragment of ANGPT1, an ANGPT1 mimic, or a nucleic acid encoding ANGPT1, or an active fragment thereof, if the subject is identified as having a risk for progressive renal decline; and/or further comprising administering to the subject TNFSF12, an active fragment of TNFSF12, a TNFSF12 mimic, or a nucleic acid encoding TNFSF12, or an active fragment thereof, if the subject is identified as having a risk for progressive renal decline; and/or further comprising administering to the subject SPARC, an active fragment of SPARC, a mimic of SPARC, or a nucleic acid encoding SPARC, or an active fragment thereof, if the subject is identified as having a risk for progressive renal decline.

7-10. (canceled)

11. The method of claim 1, wherein the human subject has impaired kidney function, diabetes, or both, wherein the diabetes is type I diabetes or type II diabetes; or wherein the human subject is non-diabetic.

12-14. (canceled)

15. The method of claim 1, wherein the level of the protective protein is determined using an immunoassay, mass spectrometry, liquid chromatography (LC) fractionation, SOMAscam, Mesoscale platform, or
electrochemiluminescence detection, wherein the immunoassay is an ELISA or a Western blot analysis; and wherein the mass spectrometry matrix assisted laser desorption ionization-time-of-flight (MALDI-TOF), inductively coupled plasma mass spectrometry (ICP-MS), triggered-by-offset, multiplexed, accurate-mass, high-resolution, and absolute quantification (TOMAHAQ), direct analysis in real time mass spectrometry (DART-MS) or secondary ion mass spectrometry (SIMS).

16-17. (canceled)

18. The method of claim 1, wherein the sample is a blood sample, a serum sample, a plasma sample, a lymph sample, a urine sample, a saliva sample, a tear sample, a sweat sample, a semen sample, a vaginal sample, a bronchial sample, a mucosal sample, or a cerebrospinal fluid (CSF) sample.

19. A protein array for identifying or monitoring progressive renal decline of a human subject, said protein array comprising antibodies or antigen-binding fragments thereof, specific for human FGF20, human TNFSF12, human ANGPT1, human Testican-2, human SPARC, human CCL5, human APP, human PF4, human ANGPT1, human DNAJC19, human TNFSF12, or combinations thereof; and/or a plurality of probes for specifically binding a protein biomarker, wherein the protein biomarker is at least one of human FGF20, human TNSF12, human ANGPT1 human Testican-2, human SPARC, human CCL5, human APP, human PF4, and human DNAJC19.

20-22. (canceled)

23. A test panel comprising the protein array of claim 19.

24. A kit or assay device comprising the test panel of claim 23.

25. (canceled)

26. A method of treating or preventing renal decline in a human subject, said method comprising
administering to a subject an effective amount of at least one protective protein and/or at least one agonist of a protective protein.

27. (canceled)

28. The method of claim 26, wherein the at least one protective protein is one or more of FGF20, TNFSF12, ANGPT1, Testican-2, SPARC, CCL5, APP, PF4, and DNAJC19; wherein at least one protective protein is FGF20, an active fragment of FGF20, a FGF20 mimic, or a nucleic acid encoding FGF20, or an active fragment thereof; and/or wherein the at least one protective protein is TNFSF12, an active fragment of TNFS12, a TNFSF12 mimic, or a nucleic acid encoding TNFSF12, or an active fragment thereof; and/or wherein the at least one protective protein is ANGPT1, an active fragment of ANGPT1, a ANGPT1 mimic, or a nucleic acid encoding ANGPT1, or an active fragment thereof; and/or wherein the at least one protective protein is SPARC, an active fragment of SPARC, a SPARC mimic, or a nucleic acid encoding SPARC, or an active fragment thereof; and/or wherein the at least one protective protein is CCL5, an active fragment of CCL5, a CCL5 mimic, or a nucleic acid encoding CCL5, or an active fragment thereof; and/or wherein the at least one protective protein is APP, an active fragment of APP, a APP mimic, or a nucleic acid encoding APP, or an active fragment thereof; and/or wherein the at least one protective protein is PF4, an active fragment of PF4, a PF4 mimic, or a nucleic acid encoding PF4, or an active fragment thereof; and/or wherein the at least one protective protein is DNAJC19, an active fragment of DNAJC19, a DNAJC19 mimic, or a nucleic acid encoding DNAJC19, or an active fragment thereof; and/or wherein the at least one protective protein is Testican-2, an active fragment of Testican-2, a Testican-2 mimic, or a nucleic acid encoding Testican-2, or an active fragment thereof.

29-37. (canceled)

38. The method of claim 28, wherein the nucleic acid is in a vector.

39. The method of claim 26, wherein the human subject was previously identified as a progressor at risk of developing progressive renal decline.

40. A method of determining the approximate risk of renal decline in a human subject in a defined time period, the method comprising:
a) obtaining a biological sample from the human subject;
b) detecting the level of at least one protective protein in the biological sample, wherein the at least one protective protein is selected from the group consisting of FGF20, TNFSF12, ANGPT1, Testican-2, SPARC, CCL5, APP, PF4, and DNAJC19;
c) combining data on the level of the protective proteins with clinical data features of the human subject (such as eGFR, uACR, Clinical Chemistry laboratory measurements, vital signs, patient demographics); and
d) determining the approximate risk of renal decline (RD) for the human subject as determined using a machine-learned or statistically modelled, prognostic risk-score algorithm (e.g., KidneyIntelX test platform).

41. The method of claim 40, further comprising comparing the level of the at least one protective protein in the biological sample to a non-progressor control level or a normoalbuminuric control level.

42. The method of claim 40, wherein the biological sample is obtained from the human subject at a first time point and a second time point, wherein the second time point is obtained from the human subject about 6 months, about 12 months, about 18 months, about 24 months, about 3 years, about 4 years, about 5 years, about 10 years or about 15 years after the first time point.

43. (canceled)

44. The method of claim 42, further comprising comparing the level of the at least one protective protein in the biological sample obtained from the human subject at a first time point to the biological sample obtained from the human subject at a second time point.

45. The method of claim 3, wherein the non-progressor is a non-diabetic human subject.

46. The method of claim 3, further comprising administering a therapy to improve kidney function if the subject is identified as having a risk for progressive renal decline; and/or further comprising administering to the subject FGF20, an active fragment of FGF20, an FGF20 mimic, or a nucleic acid encoding FGF20, or an active fragment thereof, if the subject is identified as having a risk for progressive renal decline; and/or further comprising administering to the subject ANGPT1, an active fragment of ANGPT1, an ANGPT1 mimic, or a nucleic acid encoding ANGPT1, or an active fragment thereof, if the subject is identified as having a risk for progressive renal decline; and/or further comprising administering to the subject TNFSF12, an active fragment of TNFSF12, a TNFSF12 mimic, or a nucleic acid encoding TNFSF12, or an active fragment thereof, if the subject is identified as having a risk for progressive renal decline; and/or further comprising administering to the subject SPARC, an active fragment of SPARC, a mimic of SPARC, or a nucleic acid encoding SPARC, or an active fragment thereof, if the subject is identified as having a risk for progressive renal decline.

47. The method of claim 3, wherein the human subject has impaired kidney function, diabetes, or both, wherein the diabetes is type I diabetes or type II diabetes; or wherein the human subject is non-diabetic.

48. The method of claim 3, wherein the level of the protective protein is determined using an immunoassay, mass spectrometry, liquid chromatography (LC) fractionation, SOMAscam, Mesoscale platform, or electrochemiluminescence detection, wherein the immunoassay is an ELISA or a Western blot analysis; and wherein the mass spectrometry matrix assisted laser desorption ionization-time-of-flight (MALDI-TOF), inductively coupled plasma mass spectrometry (ICP-MS), triggered-by-offset, multiplexed, accurate-mass, high-resolution, and absolute quantification (TOMAHAQ), direct analysis in real time mass spectrometry (DART-MS) or secondary ion mass spectrometry (SIMS).

49. The method of claim 3, wherein the sample is a blood sample, a serum sample, a plasma sample, a lymph sample, a urine sample, a saliva sample, a tear sample, a sweat sample, a semen sample, a vaginal sample, a bronchial sample, a mucosal sample, or a cerebrospinal fluid (CSF) sample
